SEQUENCE LISTING (1 sequence)

SEQ ID NO: 1 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

(19) United States
(12) Patent Application Publication
(10) Pub. No.: US 2011/0091447 A1
(43) Pub. Date: Apr. 21, 2011

(54) LYMPHOCYTE CONTROL OF OBESITY AND INSULIN RESISTANCE

(75) Inventors: Hans-Michael Dosch, Toronto (CA); Yin Chan, Toronto (CA); Shawn Winer, Thornhill (CA); Geoffrey Palster, Toronto (CA)

(73) Assignee: THE HOSPITAL FOR SICK CHILDREN, Toronto, ON (CA)

(21) Appl. No.: 12/922,227

(22) PCT Filed: Mar. 13, 2009
(86) PCT No.: PCT/CA2009/000318

§ 371 Date: Dec. 15, 2010

Related U.S. Application Data
(60) Provisional application No. 61/069,422, filed on Mar. 13, 2008.

(51) Int. Cl. A61K 39/395; A61P 3/00; A61P 3/10

(52) U.S. Cl. 424/130.1

(57) ABSTRACT

Methods for the treatment, inhibition, prevention, etc. of metabolic syndrome (MetSyn) and/or Type-2 diabetes (T2D) using a T cell activating antibody that stimulates activation and immunomodulation of T-cells to affect the immune environment of adipose tissue and improve insulin sensitivity. Also methods for the treatment, inhibition, prevention, amelioration, reversal, etc., of metabolic syndrome (MetSyn) and/or Type-2 diabetes (T2D) using leukemia inhibitory factor (LIF), or a functional portion thereof. Compositions administered to individuals having or at risk of developing MetSyn and/or T2D may include pharmaceutical compositions. Methods may also provide tolerogenic vaccines based on the administration of identified auto-antigens from individuals having or at risk of developing MetSyn and/or T2D.

### CROSS-REFERENCE TO RELATED APPLICATIONS

This application claims the benefit of priority to U.S. Provisional Patent Application No. 61/069,422, filed Mar. 13, 2008, entitled “LYMPHOCYTE CONTROL OF OBESITY AND INSULIN RESISTANCE THROUGH LEUKEMIA INHIBITORY FACTOR,” which is hereby incorporated by reference in its entirety.

### FIELD OF THE INVENTION

The present invention generally relates to methods for treating, preventing, inhibiting, ameliorating, reversing, etc., diet-induced obesity (DIO) and/or obesity associated metabolic derangements, such as insulin resistance, hyperinsuliunemia, glucose intolerance, liver steatosis, dyslipidemia, and progression to metabolic syndrome (MetSyn) and Type-2 diabetes (T2D). Such methods may further relate to administration of antibody compositions that trigger activation and subsequent inactivation of T-cell compartments to modulate the immunological environment of adipose tissue to improve insulin sensitivity and glucose tolerance, as well as administration of compositions containing leukemia inhibitory factor (LIF) that may achieve similar effects on glucose metabolism. In addition, methods of the present invention may further relate to tolerogenic vaccines for treating, preventing, inhibiting, etc., MetSyn and/or T2D based on tolerogenic immunotherapy with a visceral fat-selective auto-antigen associated with DIO, MetSyn, and/or T2D.

### BACKGROUND OF THE INVENTION

The incidence of obesity is increasing worldwide, and the numerous health consequences of obesity may precede and include a metabolic syndrome (MetSyn) comprised of insulin-resistance, glucose-intolerance/toxicity, hepatic steatosis, and dyslipidemia with increased risk for developing Type 2 Diabetes (T2D). See, e.g., Kahn, S. E. et al., “Mechanisms linking obesity to insulin resistance and type 2 diabetes,” Nature 444:840-846 (2006), the entire contents and disclosure of which are hereby incorporated by reference. The pathoetiology of T2D, MetSyn, and obesity may each involve genetic and environmental factors. See, e.g., Despres, J. P. et al., “Abdominal obesity and metabolic syndrome,” Nature 444:881-887 (2006), the entire contents and disclosure of which are hereby incorporated by reference. At present, although there is a link between diet-induced obesity and each of the triad components, it remains difficult if not impossible to clinically identify patients that will progress to MetSyn and/or frank T2D because very few, if any, reliable prognostic factors have been solidly identified. This triad of T2D, MetSyn, and obesity represents a major global cause of morbidity and mortality, yet current therapies for halting or reversing these progressions are inadequate. See, e.g., Dahabreh, I. J., “Meta-analysis of rare events: an update and sensitivity analysis of cardiovascular events in randomized trials of rosiglitazone,” Clin Trials 5:116-120 (2008), the entire contents and disclosure of which are hereby incorporated by reference. A need continues in the art for novel compositions and methods for the effective treatment of each of the triad components including T2D, MetSyn, and/or obesity.

### SUMMARY OF THE INVENTION

According to a first broad aspect of the present invention, a method is provided comprising the following steps: (a) identifying an individual having, or at risk of developing, metabolic syndrome (MetSyn) or Type-2 diabetes (T2D); and (b) administering to the individual a therapeutically effective amount of a composition comprising a T cell activating antibody.

According to a second broad aspect of the present invention, a method is provided comprising the following steps: (a) identifying an individual having, or at risk of developing, metabolic syndrome (MetSyn) or Type-2 diabetes (T2D); and (b) administering to the individual a therapeutically effective amount of a composition comprising a leukemia inhibitory factor (LIF) protein or a functional portion thereof.

According to a third broad aspect of the present invention, a use is provided of a composition comprising a T cell activating antibody, to treat an individual having, or at risk of developing, metabolic syndrome (MetSyn) or Type-2 diabetes (T2D.

According to a fourth broad aspect of the present invention, a use is provided a leukemia inhibitory factor (LIF) protein or a functional portion thereof, to treat an individual having, or at risk of developing, metabolic syndrome (MetSyn) or Type-2 diabetes (T2D).

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1A is a set of fluorescent-activated cell sorting (FACS) plots showing CD3+ gated visceral adipose tissue (VAT) and subcutaneous adipose tissue (SAT) stromal vascular cells and splenocytes from 14-16 week old regular diet and DIO B6 mice (from 3-5 independent experiments) grouped into quadrants based on CD4 and CD8 staining with percentages of cells within each quadrant shown;

FIG. 1B is a set of FACS plots showing the proportions of CD4+ T-cells that are IFNγ+ (Th1) and IL-17+ (Th17) in spleen, SAT, and VAT;

FIG. 1C is a set of FACS plots showing the proportions of CD4+Foxp3+ (Treg) cells in spleen, SAT, and VAT in the upper panel, with the lower panel showing bar graphs of pooled data (n=4-5/group; p<0.03, Welch-test);

FIG. 1D is a set of bar graphs showing the total number (×10³) of CD3+/CD4+ cells (upper left), CD4+/IFNγ+ (TH1) cells (lower left), CD4+/IL-17+ (Th17) cells (upper right), and CD4+/Foxp3+ (Treg) cells (lower right) per gram of SAT and VAT (pooled data; n=4-5/group; *p<0.05, Welch-test);

FIG. 1E includes a plot in the left panel showing the correlation of T-bet:Foxp3 ratios (measured from at least 200 cells at two different tissue locations of human VAT) to BMI (r²=0.62, p<0.05, Welch-test) with the insert of the left panel showing that T-bet:Foxp3 ratio is elevated in patients with BMI>30 compared to patients with BMI<25, and microscopic (200× magnification) images of VAT tissue sections in the right two panels from a patient with BMI=35.7 (upper image) and from a patient with BMI=24 (lower image) stained for Foxp3 (brown) and T-bet (blue);

FIG. 1F is a set of FACS plots of CD3+/CD4+ gated cells showing the percentage of cells (in boxed portions) with secondary rearranged non-OVA TCRα (i.e., with a reduced OT2 TCRα:CD3 ratio) from spleen, SAT, and VAT of 6 week old B6 OT2 mice and regular diet or DIO 16 week old B6 OT2 mice;

FIG. 1G is a set of images of agarose gels showing PCR products of TCRVα gene rearrangement clones of CD4+ T cells isolated from spleens of 16 week old DIO wild type (lower middle panel) and 16 week old DIO OT2 mice (top panel) as well as VAT from 16 week old DIO wild type (bottom panel) and 16 week old DIO OT2 mice (upper middle panel) with CD4+ T-cells from DIO OT2 mice sorted for a reduced OT2 TCRα:CD3 ratio (i.e., sorted to increase T-cells with rearranged TCRα);

FIG. 1H is a spectra-typing plot for Vα5 and Vα18 of TCRVα showing T-cell clonality in spleen and VAT of DIO OT2 mice;

FIG. 2A is a microscopic image of T-bet labeled cells and Foxp3+ cells scattered throughout human adipose tissue at 200× magnification;

FIG. 2B is a microscopic image of Foxp3+ cells in close proximity to CD14+ antigen presenting cells at 400× magnification;

FIG. 3A is a graph showing body weights of wild-type B6 or B6.OT2 mice on either a regular diet (RD) or high fat diet (DIO) depending on age in weeks of mice (n=5, *p<0.05, Welch-test on each time point);

FIG. 3B is a graph showing glucose tolerance of wild-type B6 or B6.OT2 mice on either a regular diet (RD) or high fat diet (DIO) over time in minutes;

FIG. 3C is a bar graph showing fasting insulin levels in wild-type B6 or B6.OT2 mice on either a regular diet (RD) or high fat diet (DIO);

FIGS. 4, 4-1, 4-2, and 4-3 together are spectra-typing plots for TCRVα clones including Vα1, Vα2, Vα8, Vα10, and Vα11 from T-cells expressing a rearranged TCRα in spleen or VAT of DIO OT2 mice;

FIG. 5A is a bar graph of body weights of WT and RAG^{null} on either a regular fat diet (RD, n=20/group) or a high fat diet (DIO, n=20/group) beginning at about 6 weeks of age (*, p<0.03, Mann-Whitney test) with statistical comparison between WT and RAG^{null} mice of similar diet;

FIG. 5B is a bar graph showing fat mass of single epididymal adipose fat pads (VAT) and inguinal fat pads (SAT) from 14-15 week old WT and RAG^{null} mice on a regular diet (RD) or high fat diet (DIO) (left panel, n=6 mice/group, *p<0.03, Welch-test) and showing VAT:SAT ratios from these measurements in the right panel (*p<0.001, Welch-test);

FIG. 5C is a plot showing the cell diameter distributions of adipocytes in VAT and SAT from WT and RAG^{null} mice on a regular diet (RD) or high fat diet (DIO) (n=3 mice/group, *p<0.0001, Mann-Whitney test);

FIG. 5D is a set of bar graphs showing a comparison of food intake (top panel) and respiratory exchange ratio (RER, bottom panel) between WT and RAG^{null} mice on a high fat diet (n=4/group);

FIG. 5E is a plot showing glucose tolerance over time of WT and RAG^{null} mice on a regular diet (RD) or high fat diet (DIO) with statistical comparison between WT and RAG^{null} mice of similar diet (n=10/group, *p<0.02, two-way ANOVA);

FIG. 5F is a set of bar graphs showing fasting glucose levels (left panel) and fasting insulin levels (right panel) in 14 week old WT and RAG^{null} mice on a regular diet (RD) or high fat diet (DIG) with statistical comparison between WT and RAG^{null} mice of similar diet (n=10 mice/group, *p<0.05, Welch-test);

FIG. 5G is a plot showing the percentage changes in glucose levels over time in an insulin tolerance test (ITT) following injection of an insulin challenge dose between 14 week old WT and RAG^{null} mice on either RD or high fat diet (DIO) (n=6-10 mice/group) with statistical comparison between DIO WT and RAG^{null} mice, but not RD groups (*p<0.05, two-way ANOVA);

FIG. 6A is a FACS plot showing the purity of CD3+/CD8+ (top) and CD3+/CD4+ (bottom) T-cells from spleen, SAT, and VAT of mice 2 weeks following transfer of purified CD4+ or CD8+ T-cells into 12 week old DIO RAG^{null} recipients;

FIG. 6B is a bar graph showing body weights (n=5/group) of recipient mice 0, 2, and 4 weeks after transfer of CD4+ or CD8+ cells into 12 week old DIO RAG^{null} recipients compared to non-transferred, age-matched DIO RAG^{null} mice (*p<0.05, Welch-test) with one of four similar experiments shown;

FIG. 6C is a bar graph showing fat mass of individual epididymal (VAT) and inguinal (SAT) fat pads of DIO RAG^{null} recipient mice 2-4 weeks after transfer of CD4+ or CD8+ T-cells compared to non-transferred, age-matched DIO RAG^{null} mice (n=5 mice/group, *p<0.05, Welch-test);

FIG. 6D is a plot showing the cell diameter distributions of adipocytes in VAT and SAT from DIO RAG^{null} recipient mice 2-4 weeks after transfer of CD4+ or CD8+ cells compared to non-transferred, age-matched DIO RAG^{null} mice (n=3 mice/group, *p<0.01, Mann-Whitney test);

FIG. 6E is a plot of glucose levels over time in a glucose tolerance test (OTT) following glucose injection in DIO RAG^{null} recipient mice after transfer of CD4+ or CD8+ cells compared to non-transferred, age-matched DIO RAG^{null} mice (n=10 mice/group, *p<0.0001, two-way ANOVA);

FIG. 6F is a set of bar graphs showing the fasting glucose levels (left panel) and fasting insulin levels (right panel) in CD4+ or CD8+ transferred DIO-RAG^{null} mice compared to non-transferred, age-matched DIO RAG^{null} mice (n=5-10 mice/group, *p<0.03, **p<0.01, Welch-test);

FIG. 6G is a plot showing the percentage changes in glucose levels over time in an insulin tolerance test (ITT) following injection of an insulin challenge dose between CD4+ or CD8+ transferred DIO-RAG^{null} mice compared to non-transferred, age-matched DIO RAG^{null} mice (n=10, *p<0.05, two-way ANOVA);

FIG. 6H is a plot showing glucose tolerance over time following glucose challenge in DIO-RAG^{null} recipient mice transferred with CD4+ or CD4+OT2:TCRα^{hi} (CD4+OT2^{hi}) T-cells compared to non-transferred, age-matched DIO RAG^{null} mice (n=5 mice/group, *p<0.0001, two-way ANOVA);

FIG. 6I is a plot showing the percentage changes in glucose levels over time in an insulin tolerance test (ITT) following injection of an insulin challenge dose in DIO-RAG^{null} recipient mice transferred with CD4+ or CD4+OT2:TCRα^{hi} (CD4+OT2^{hi}) T-cells compared to non-transferred, age-matched DIO RAG^{null} mice (n=5 mice/group, *p<0.0001, two-way ANOVA);

FIG. 6J is a bar graph showing fasting glucose levels in DIO-RAG^{null} recipient mice transferred with CD4+ or CD4+OT2:TCRα^{hi} (CD4+OT2^{hi}) T-cells compared to non-transferred, age-matched DIO RAG^{null} mice (n=5 mice/group, *p<0.05, Welch-test);

FIG. 6K is a bar graph showing fasting insulin levels in DIO-RAG^{null} recipient mice transferred with CD4+ or CD4+OT2:TCRα^{hi} (CD4+OT2^{hi}) T-cells compared to non-transferred, age-matched DIO RAG^{null} mice (n=5 mice/group, *p<0.05, Welch-test);

FIG. 6L is a bar graph showing changes in body weights of DIO-RAG^{null} recipient mice following transfer of CD4+ or CD4+OT2:TCRα^{hi} (CD4+OT2^{hi}) T-cells compared to non-transferred, age-matched DIO RAW″ mice (n=5 mice/group, *p<0.01, Welch-test);

FIG. 6M is a set of FACS plots showing percentages of OT2 TCRVα rearrangements in spleen and VAT 2 weeks following adoptive transfer of CD4+ OT2:TCRα^{hi} lymphocytes into RAG^{null} mice;

FIG. 7 is a set of microscopic images at 100× magnification showing representative H&E staining of tissue from SAT, VAT, and liver from 14-16 week old DIO-RAG^{null} mice reconstituted with either 5×10⁶ CD4+ or CD8+ T-cells compared to non-transferred, age-matched DIO RAG^{null} or DIO WT mice;

FIG. 8 is a bar graph (upper panel) comparing food intake of DIO-RAG^{null} mice one week following transfer of 5×10⁶ CD4+ T-cells compared to non-transferred, age-matched DIO RAG^{null} mice, and a set of plots (middle and lower panels) comparing O₂ consumption and CO₂ output of DIO-RAG^{null} mice one week following transfer of 5×10⁶ CD4+ T-cells compared to non-transferred, age-matched DIO RAG^{null} mice (n=4 mice/group);

FIG. 9 is a bar graph (upper panel) comparing food intake of DIO-RAG^{null} mice two weeks following transfer of 5×10⁶ CD4+ T-cells compared to non-transferred, age-matched DIO RAG^{null} mice, and a set of plots (middle and lower panels) comparing O₂ consumption and CO₂ output of DIO-RAG^{null} mice two weeks following transfer of 5×10⁶ CD4+ T-cells compared to non-transferred, age-matched DIO RAG^{null} mice (n=4 mice/group);

FIG. 10 is a set of bar graphs showing serum levels of adiponectin, leptin, resistin, MCP-1, IL-6, and TNF-α from 16 week old DIO RAG^{null} mice reconstituted with either 5×10⁶ CD4+ or CD8+ T-cells compared to non-transferred, age-matched DIO RAG^{null} or DIO WT mice (n=3-6 mice/bar, *p<0.05, Welch-test);

FIG. 11A is a set of FACS plots showing percentages of CD4+/Foxp3+ T-cells in spleen and VAT 3 weeks following transfer of purified CD4+ or purified CD4+Foxp3-(EGFP^{neg}; 99.1% pure on initial transfer) cells with one of two similar FACS plots shown, and “0” is used to show an absence of detectable CD4+/Foxp3+ T-cells;

FIG. 11B is a bar graph showing changes in body weight of DIO-RAG^{null} recipients 3 weeks post transfer of CD4+, CD4+/Foxp3−, or CD4+/IL-10^{null} T cells compared to non-transferred, age-matched RAG^{null} mice (n=5 mice/group, *p<0.01, Welch-test);

FIG. 11C is a plot of glucose levels over time in a glucose tolerance test (GTT) following glucose injection in DIO-RAG^{null} recipients 3 weeks post transfer of CD4+, CD4+/Foxp3−, or CD4+/IL-10^{null} T cells compared to non-transferred, age-matched RAG^{null} mice (n=5 mice/group, *p<0.0001, two-way ANOVA);

FIG. 11D is a bar graph showing fasting glucose levels in DIO-RAG^{null} recipients 3 weeks post transfer of CD4+, CD4+/Foxp3−, or CD4+/IL-10^{null} T-cells compared to non-transferred, age-matched RAG^{null} mice (n=5 mice/group, *p<0.05, Welch-test);

FIG. 11E is a bar graph showing fasting insulin levels in DIO-RAG^{null} recipients 3 weeks post transfer of CD4+, CD4+/Foxp3−, or CD4+/IL-10^{null} T-cells compared to non-transferred, age-matched RAG^{null} mice (n=5 mice/group, *p<0.05, Welch-test);

FIG. 11F is a bar graph showing IL-4 levels produced by T-cells purified from VAT of 16 week old regular diet (RD) WT mice, DIO WT mice, DIO RAG^{hull} mice, and DIO RAG^{null} recipients 3 week post transfer of CD4+ T cells following stimulation with anti-CD3 and anti-CD28 antibodies (n=3 mice/group);

FIG. 11G is a bar graph showing IL-13 levels produced by T-cells purified from VAT of 16 week old regular diet (RD) WT mice, DIO WT mice, DIO RAG^{null} mice, and DIO RAG^{null} recipients 3 week post transfer of CD4+ T cells following stimulation with anti-CD3 and anti-CD28 antibodies (n=3 mice/group);

FIG. 11H is a plot of glucose levels over time in a glucose tolerance test (GTT) following glucose injection in DIG RAG^{null} recipient mice 3 weeks after transfer of CD4+ or CD4+/STAT6^{-/-} cells compared to non-transferred, age-matched DIO RAG^{null} mice (n=5 mice/group, *p<0.05, two-way ANOVA);

FIG. 11I is a bar graph showing fasting glucose levels in DIO RAG^{null} recipient mice 3 weeks after transfer of CD4+ or CD4+/STAT6^{-/-} cells compared to non-transferred, age-matched DIO RAG^{null} mice (n=5 mice/group, *p<0.01, Welch-test);

FIG. 11J is a bar graph showing fasting insulin levels in DIO RAG^{null} recipient mice 3 weeks after transfer of CD4+ or CD4+/STAT6^{-/-} cells compared to non-transferred, age-matched DIO RAG^{null} mice (n=5 mice/group, *p<0.01, Welch-test);

FIG. 11K is a bar graph showing weight change in DIO RAG^{null} recipient mice 3 weeks after transfer of CD4+ or CD4+/STAT6^{-/-} cells compared to non-transferred, age-matched DIO RAG^{null} mice (n=5 mice/group, *p<0.01, Welch-test);

FIG. 12A is a FACS plot showing percentages of CD4+/Foxp3+ (Treg) T cell populations from spleen or VAT of 16 week old DIG B6 mice (on a high fat diet for 9 weeks) after treatment with either anti-CD3 or isotype control antibody;

FIG. 12B is a set of bar graphs showing fasting glucose levels (upper panel) and fasting insulin levels (lower panel) in DIO B6 mice (on a high fat diet for 9 weeks) after treatment with either anti-CD3 or isotype control antibody (n=8, *p<0.05, Welch-test);

FIG. 12C is a plot showing glucose tolerance in DIO B6 mice (on a high fat diet for 9 weeks) after treatment with either anti-CD3 or isotype control antibody (n=8, *p<0.0004, two-way ANOVA);

FIG. 12D is a plot showing the percentage changes in glucose levels over time in an insulin tolerance test (ITT) following injection of an insulin challenge dose in DIO B6 mice (on a high fat diet for 9 weeks) after treatment with either anti-CD3 or isotype control antibody (n=8, *p<0.0005, two-way ANOVA);

FIG. 12E is a plot of weekly body weight measurements of DIO B6 mice (on a high fat diet for 9 weeks) following treatment with either anti-CD3 or isotype control antibody (n=8, *p<0.05, Welch-test);

FIG. 12F is a plot showing the cell diameter distributions of adipocytes from DIO B6 mice (on a high fat diet for 9 weeks) following treatment with either anti-CD3 or isotype control antibody;

FIG. 12G is a plot of glucose levels over time in a glucose tolerance test (MT) following glucose injection in DIO B6 mice (on a high fat diet for 6 weeks) following treatment with either F(ab′)₂ fragment of the anti-CD3 antibody or isotype control antibody (n=5, *p<0.01, two-way ANOVA) with one of 2 similar experiments is shown;

FIG. 12H is a bar graph showing fasting insulin levels (upper panel) and fasting glucose levels (lower panel) in DIO B6 mice (on a high fat diet for 6 weeks) following treatment with either F(ab′)₂ fragment of the anti-CD3 antibody or isotype control antibody (n=5, *p<0.05, Welch-test);

FIG. 12I is a plot of weekly measurements of body weights of DIO B6 mice (on a high fat diet for 6 weeks) following treatment with either F(ab′)₂ fragment of the anti-CD3 antibody or isotype control antibody (n=5 mice/group);

FIG. 12J is a set of FACS plots showing the percentages of CD4+/Foxp3+ (Treg) T-cell populations in spleen or VAT from DIO B6 mice 6 weeks after treatment with F(ab′)₂ fragment of the anti-CD3 antibody or isotype control antibody;

FIG. 12K is a set of bar graphs showing the levels of IFNγ, IL-17, IL-13, and IL-4 levels produced by T-cells purified from VAT of either isotype control- or F(ab′)₂-treated DIO B6 mice following stimulation with anti-CD3 and anti-CD28 antibodies (6 week post treatment; n=3 mice/group);

FIG. 13 is a bar graph (upper panel) comparing 24 hour food intake of 23 week old DIO B6 mice 9 weeks following treatment with either anti-CD3 or isotype control antibodies, and a set of plots (middle and lower panels) comparing O₂ consumption and CO₂ output of 23 week old DIO B6 mice 9 weeks following treatment with either anti-CD3 or isotype control antibodies;

FIG. 14A is a plot showing glucose levels over time in a glucose tolerance test (GTT) following glucose injection in 16 week old WT B6 mice 2 weeks after injection of anti-CD3 or isotype control antibodies (n=5 mice/group);

FIG. 14B is a plot showing glucose levels over time in a glucose tolerance test (OTT) following glucose injection in 16 week old WT B6 mice 4 weeks after injection of anti-CD3 or isotype control antibodies (n=5 mice/group, *p<0.03, two-way ANOVA);

FIG. 14C is a plot showing glucose levels over time in a glucose tolerance test (GTT) following glucose injection in 16 week old WT B6 mice 12 weeks after injection of anti-CD3 or isotype control antibodies (n=5 mice/group, *p<0.03, two-way ANOVA);

FIG. 14D is a plot showing glucose levels over time in a glucose tolerance test (GTT) following glucose injection in 16 week old WT B6 mice 16 weeks after injection of anti-CD3 or isotype control antibodies (n=5 mice/group, *p<0.03, two-way ANOVA);

FIG. 15A is a set of FACS plots showing the proportions of MMR^{hi} (upper gate), MMR^{lo} (middle gate), and MMR^{neg} (lower gate) macrophages from VAT of 16 week old DIO or regular diet B6 mice with each plot representative of 4 experiments;

FIG. 15B is a set of bar graphs showing the amounts of IL-10 (left panels), MCP-1 (middle panels) and TNFα (right panels) produced by LPS stimulated, F4/80+ macrophages from VAT of DIO (top panels) or regular diet (lower panels) mice sorted into MMR^{neg}, MMR^{lo}, and MMR^{hi} cell populations with one of 4 similar experiments shown;

FIG. 15C is a set of FACS plots showing the proportions of MMR^{hi} (upper gate), MMR^{lo} (middle gate), and MMR^{neg} (lower gate) of F4/80+ macrophages from VAT of DIO B6 mice at 6 weeks after treatment with either F(ab′)₂ fragment of anti-CD3 antibody or isotype control antibody;

FIG. 15D is a set of bar graphs showing the amounts of IL-10 (left panel), MCP-1 (middle panel) and TNFα (right panel) produced by LPS stimulated, F4/80+ macrophages from VAT 6 weeks after treatment with either F(ab′)₂ fragment of anti-CD3 antibody or isotype control antibody (n=3 mice/group, *p<0.05, Welch-test) with each graph representative of 2 similar experiments;

FIG. 16A is a time plot comparing glucose levels in RAG^{null} mice following injection with either anti-CD3 or control isotype antibody;

FIG. 16B is a time plot comparing glucose levels in B cell^{null} (IgM^{null}) mice following injection with either anti-CD3 or control isotype antibody;

FIG. 16C is a time plot comparing glucose levels in B cell-activated (IgM treated) mice following injection with either anti-CD3 or control isotype antibody;

FIG. 16D is a time plot comparing glucose levels in wild-type (WT) or MHC class I (CD8-deficient) (β2 m^{null} mice each following injection with either anti-CD3 or control isotype antibody;

FIG. 16E is a time plot comparing glucose levels in wild-type (WT) or MHC class I (CD4-deficient) (c2t^{null}) mice each following injection with either anti-CD3 or control isotype antibody;

FIG. 17 is a time plot comparing non-fasting glucose levels in RAG^{null} mice injected i.p. with serum (400 μl) obtained from donor mice 4 hours after injection of donor mice with anti-CD3 or isotype control antibodies (n=3-4 mice/group. *p<0.05, Welch-test);

FIG. 18A is a bar graph showing a list of secretory genes identified by a gene array experiment having more than a 5-fold increase in expression following anti-CD3 treatment with IL-6, LIF, and Retnlg highlighted in black as candidates;

FIG. 18B is a bar graph showing the amount of LIF produced in vitro by purified CD4+ and CD8+ T-cells from spleens of 16 week old DIO WT mouse following stimulation with plate-bound anti-CD3 antibody (1 μg/ml) and anti-CD28 antibody (0.25 mg/ml) (n=3 mice/group);

FIG. 18C is a time plot showing the amount of LIF in serum of mice following treatment with anti-CD3 antibody (n=3 mice/time point) with insert showing neutralization of LIF production for at least 10 hours following injection with a polyclonal anti-LIF antibody (*p<0.05, Welch-test);

FIG. 18D is a time plot showing blood glucose levels following anti-CD3 injection with anti-LIF or control isotype antibodies injected 2.5 hours after anti-CD3 injection as indicated by the arrow (n=4 mice, *p<0.05, Welch-test);

FIG. 18E is a time plot of non-fasting blood glucose levels in 8 week old WT B6 mice following injection with 1 μg or 5 μg of LIF (n=3 mice, *p<0.05 relative to starting glucose levels, Welch-test);

FIG. 18F is a bar graph showing levels of LIF produced by T^{fat} cells (from SAT) or splenocytes cultured in vitro from DIO WT or ob/ob mice with (+) or without (−) stimulation with plate-bound anti-CD3 antibody (1 ng/ml) and anti-CD28 antibody (0.25 μg/ml);

FIG. 18G is a plot of enhanced LIF production by in vitro cultured T^{fat} cells stimulated with anti-CD3 antibody (1 μg/ml) with different levels of IL-1β;

FIG. 18H is an image of a Western blot for total protein from SAT (lanes 1-4) or VAT (lanes 5-8) from 15 week old DIO WT mice (lanes 1, 2, 5, and 6) or DIO-RAG^{null} mice (lanes 3, 4, 7, and 8) probed with anti-LIF antibody;

FIG. 18I is a FACS plot showing LIF producing CD8+ (top left; solid line) and CD4+ (top right; solid line) T-cells purified from DIO WT spleen or LIF producing CD4+ cells (bottom panel) that are positive (solid line) or negative (dotted line) for IFNγ after stimulation with anti-CD3 antibody for about 12 hours compared to unstimulated cells (shaded region in each panel) with results representative of 3 similar experiments;

FIG. 18J is a set of microscopic images at 400× magnification of human tonsil tissue (left panel) and human VAT (right panel) showing separate color staining of CD3+ cells and LIF+ cells with cells producing both CD3 and LIF either displaying a merged color or the CD3 color surrounding the LIF color in the interior of the cell;

FIG. 18K is a plot of glucose levels over time in a glucose tolerance test (GTT) following glucose injection in 15 week old DIO WT mice after 1 week of injections of neutralizing anti-LIF antibody or isotype control antibody (i.e., 300 μg on day 0, then 150 ng on days 3 and 6) (n=5 mice/group, p=0.05, two-way ANOVA, *p<0.04, Welch-test).

FIG. 18L is a plot of glucose levels over time in a glucose tolerance test (GTT) following glucose challenge in 15 week old DIO WT mice after 2 week of injections of neutralizing anti-LIF antibody or isotype control antibody (i.e., 300 μg on day 0, then 150 μg on days 3 and 6 of each week) (n=5 mice/group, p=0.05, two-way ANOVA, *p<0.04, Welch-test).

### DETAILED DESCRIPTION

### Definitions

Where the definition of tennis departs from the commonly used meaning of the term, applicant intends to utilize the definitions provided below, unless specifically indicated.

For purposes of the present invention, the twin “antigen” in reference to a T cell activating antibody is a macromolecule, such as a protein, polysaccharide, etc., or a complex of macromolecules that is specifically bound by the T cell activating antibody. For example, such antigen may be a component(s) or subunit(s) of a T-cell receptor complex or an accessory molecule(s) of a T-cell, or any subunit or portion thereof, which may be present on the surface of a T-cell. The term “antigen” may also refer to a molecule or substance that is used as an immunogen injected into an animal to cause the production of antibodies that specifically bind to the antigen.

For purposes of the present invention, the term “epitope” refers to a region(s) of a macromolecule(s) that is/are bound by an antibody or an immunoglobulin-like polypeptide, or a portion or fragment thereof. The term “epitope” may also be referred to as an antigenic determinant.

For purposes of the present invention, the term “antibody” refers to one or more immunoglobulin or immunoglobulin-like polypeptide(s), or portion(s) or fragment(s) thereof, which retain the ability to bind to an antigen. Such an antibody may include a complex of immunoglobulin or immunoglobulin-like polypeptide(s), which may be linked together by one or more disulfide linkages, or a single immunoglobulin or immunoglobulin-like polypeptide, such as a scFv, with each immunoglobulin or immunoglobulin-like polypeptide having at least a portion of one or more immunoglobulin domain(s). Such an immunoglobulin domain(s) will generally include at least a portion of a variable region or domain responsible for specific binding to an antigen. Such an antibody may include at least a portion of a heavy chain and at least a portion of a light chain, which may be covalently linked together to form a single chain. Such an antibody may include polyclonal or monoclonal antibodies. Such an antibody may include a full sized antibody of any type (e.g., IgG, IgM, IgA, IgE, IgD, etc.) or a fragment of an antibody, such as Fab or F(ab′)₂. Such an antibody may include any immunoglobulin or immunoglobulin-like polypeptide, or fragment thereof, which has been engineered, for example, by recombinant techniques to have desired binding properties or expression characteristics, and such an antibody may include any antibody encoded by a polynucleotide sequence that has been subjected to mutagenesis. Further, such an antibody may include chimeric or humanized antibodies including, for example, monoclonal antibodies engineered to have all or a portion of a human Fc constant region.

For purposes of the present invention, the term “immunoglobulin-like polypeptide” refers to any portion, fragment, or engineered version of an antibody or immunoglobulin polypeptide having at least a portion of one or more immunoglobulin domains that retains the ability to bind to an antigen. Such an immunoglobulin-like polypeptide may include at least portion of one or more immunoglobulin domains derived from the variable region or domain of a heavy and/or light chain(s) of an antibody. In general, such an immunoglobulin-like polypeptide may include any protein created by digestion, chemical treatment, recombinant techniques, etc., of another antibody or immunoglobulin-like polypeptide according to any known or improved method. For example, such an immunoglobulin-like polypeptide may include a Fab or F(ab′)₂ fragment(s) of an antibody or a scFv.

For purposes of the present invention, the term “chimeric” refers to an antibody wherein different portions of the antibody are derived from different animal species. Methods for producing chimeric antibodies are known in the art. For purposes of the present invention, the term “humanized” refers to an antibody having one or more complementarity-determining regions (CDRs) from a non-human species but a constant region from a human immunoglobulin peptide.

For purposes of the present invention, the term “T cell activating antibody” refers to an antibody that binds to an epitope present on a component(s) or subunit(s) of a T-cell receptor complex or an accessory molecule(s) of a T-cell lymphocyte. Such a “T cell activating antibody” may include any antibody that is able to trigger T-cell activation at least transiently upon binding to a component(s) or subunit(s) of a T-cell receptor complex or an accessory molecule(s) of a T-cell lymphocyte.

For purposes of the present invention, the tennis “T-cell receptor” or “TCR” refer to a complex of integral membrane proteins that are expressed on T-cell lymphocytes and together bind specifically to an antigen or MHC-antigen. Such a complex of integral membrane proteins of the TCR include the TCRα and TCRβ subunits, which form a heterodimer and may bind directly to an epitope or MHC-antigen complex. Such a complex of integral membrane proteins of the TCR may also include CD3 molecule(s), which may each be composed of δ, ε, and/or γ subunits. Such a complex of integral membrane proteins of the TCR may further include a ζ chain, which may form a homodimer with another ζ chain or a heterodimer with a η chain.

For purposes of the present invention, the term “accessory molecule” in reference to a T-cell or T-cell receptor is any protein or macromolecule present on the surface of a T-cell that is not a permanent member of a TCR complex but which may associate with a TCR complex and/or bind to molecules or ligands present on an opposing surface of a cell, tissue, or extracellular matrix. The term “accessory molecule” may also refer to such a protein or macromolecule that has been chemically removed or isolated from a T-cell or a different cell type expressing such a protein or macromolecule. The term the term “accessory molecule” may further refer to such a protein or macromolecule that has been expressed by a T-cell or other cell type using a recombinant construct containing a sequencing encoding the accessory molecule. For example, the term “accessory molecule” may include molecules or proteins, such as CD8 and CD4, respectively, that bind to class I or class II major histocompatibility complex (MHC) molecule(s), especially upon binding of a MHC-presented antigen to a TCR. The term “accessory molecule” may also include, for example, any molecule, such as CD28, etc., that provides a co-stimulation signal when present on the surface of a T-cell and bound by its ligand.

For purposes of the present invention, the term “individual” may refer to any mammal in need of treatment, inhibition, prevention, amelioration, reversing, etc., of obesity, metabolic syndrome (MetSyn), and/or Type-2 diabetes (T2D) or a condition or disease resembling obesity, MetSyn, and/or T2D. Such an individual may be a human having, or at risk of developing, obesity, MetSyn, and/or T2D.

For the purposes of the present invention, the terms “carrier” or “pharmaceutically acceptable carrier” refer interchangeably to substances that may be formulated with a T cell activating antibody or LIF, or a functional portion or fragment thereof, to make a pharmaceutical composition that may be administered according to embodiments of the present invention.

For the purposes of the present invention, the terms “functional fragment” or “functional portion” in reference to a LIF protein or peptide refer to fragments or portions of such a LIF protein or peptide that retains all or a portion of the biological activity of the full-length LIF protein or peptide.

For the purposes of the present invention, the terms “homology” and “homologous” for protein or peptide sequences refer to protein or peptide sequences having a certain degree of identity or similarity.

For the purposes of the present invention, the terms “identity” and “identical” for protein or peptide sequences refer to protein or peptide sequences having a certain degree of identity. “Identity” and “identical” may defined quantitatively, such as by a percentage or range of percentages, to specify an amount of identity. For example, protein or peptide sequences that are at least 80% identical (or protein or peptide sequences having at least 80% identity) may be defined as protein or peptide sequences having at least 80% of their sequence by position being the same (i.e., composed of the same amino acids).

For the purposes of the present invention, the terms “similarity” and “similar” for protein or peptide sequences refer to protein or peptide sequences having a certain degree of similarity, such as in terms of chemical properties of amino acid side chains. “Similarity” and “similar” may defined quantitatively, such as by a percentage or range of percentages, to specify an amount of similarity. For example, protein or peptide sequences that are at least 80% similar (or having at least 80% similarity) may be defined as protein or peptide sequences having at least 80% of their protein or peptide sequence by position being functionally similar in terms of the chemical properties of amino acid side chains (i.e., corresponding amino acids of two or more protein or peptide sequences are similar if their side chains may be grouped together chemically in terms of their polarity, hydrophobicity, charge, etc.).

### Description

The elements of metabolic syndrome (MetSyn) including insulin-resistance, glucose-intolerance/toxicity, hepatic steatosis, and dyslipidemia, likely contribute to persistent β-cell stress and dysfunction as well as risk for developing Type-2 diabetes (T2D). However, progression of MetSyn to overt diabetes is not easily predicted, and many patients with MetSyn may never convert while some may only convert transiently. See, e.g., Despres, J. P. et al., supra (2006); and Hanson, R. L., et al., “Components of the ‘metabolic syndrome’ and incidence of type 2 diabetes,” Diabetes 51:3120-3127 (2002), the entire contents and disclosures of which are hereby incorporated by reference. It remains unclear what limits or halts progression toward T2D in some patient groups with persistent MetSyn or what drives progression of MetSyn in other groups who do convert to T2D.

Obesity-associated insulin-resistance can be a core element of T2D development. The impairment of insulin-sensitivity may involve multiple organs, which may prominently include increased adipose tissue (particularly visceral fat) and an associated rise in fatty acids, adipokines, and pro-inflammatory molecules, such as IL-6 and TNFα. See, e.g., Kahn, S. E., et al., supra (2006); and Hotamisligil, G. S., “Inflammation and metabolic disorders,” Nature 444:860-867 (2006), the entire contents and disclosures of which are hereby incorporated by reference. Although TNFα can be produced by adipocytes, TNFα is largely macrophage derived. Macrophages may accumulate in both visceral adipose tissue (VAT) and subcutaneous adipose tissue (SAT) and may play distinct roles in each of these tissues in obesity-induced insulin resistance. See, e.g., Kanda, H. et al., “MCP-1 contributes to macrophage infiltration into adipose tissue, insulin resistance, and hepatic steatosis in obesity,” J Clin Invest 116:1494-1505 (2006); Yuan, M. et al., “Reversal of obesity- and diet-induced insulin resistance with salicylates or targeted disruption of Ikkbeta,” Science 293:1673-1677 (2001); Nishimura, S. et al., “In vivo imaging in mice revels local cell dynamics and inflammation in obese adipose tissue,” J Clin Invest 118:710-21 (2008); and Weisberg, S. P. et al., “CCR2 modulates inflammatory and metabolic effects of high-fat feeding,” J Clin Invest 116:115-124 (2006), the entire contents and disclosures of which are hereby incorporated by reference.

Macrophage activation may be modified by T-cells. IFNγ-secreting (Th1) as well as IL-17-secreting (Th17) T-cells generally enhance macrophage pro-inflammatory functions with release of IL-1, IL-6, and TNFα. In contrast, regulatory or anti-inflammatory IL-4 and IL-13-secreting (Th2) as well as CD4+Foxp3+ T-cells counter-regulate macrophage function by skewing macrophage differentiation into anti-inflammatory IL-10-secreting “alternatively activated” macrophages (AAM). See, e.g., Odegaard, J. I. et al., “Macrophage-specific PPAR-gamma controls alternative activation and improves insulin resistance,” Nature 447:1116-1120 (2007); and Tiemessen, M. M. et al., “CD4+CD25+Foxp3+ regulatory T cells induce alternative activation of human monocytes/macrophages,” PNAS USA 104:19446-19451 (2007), the entire contents and disclosures of which are hereby incorporated by reference. AAM are characterized by abundant surface expression of macrophage mannose receptor (MMR) and intracellular arginase activity. See, e.g., Martinez, F. O. et al., “Macrophage activation and polarization,” Front Biosci 13:453-461 (2008), the entire contents and disclosure of which are hereby incorporated by reference. Increased levels of IL-10 improve insulin sensitivity in liver and fat, and AAM in adipose tissue (AAM^{fat}) normalize some of the metabolic abnormalities associated with diet induced obesity in animal models. See, e.g., Odegaard, J. I. et al., “Macrophage-specific PPARgamma controls alternative activation and improves insulin resistance,” Nature 447:1116-1120 (2007); Cintra, D. E. et al., “Interleukin-10 is a protective factor against diet-induced insulin resistance in liver,” J Hepatol 48:628-637 (2008); and Lumeng, C. N. et al., “Obesity induces a phenotypic switch in adipose tissue macrophage polarization,” J Clin Invest 117:175-184 (2007), the entire contents and disclosures of which are hereby incorporated by reference.

CD4+ and CD8+ T-cells enter VAT and SAT of obese mice and humans, perhaps due to intrinsic tissue hypoxia. See, e.g., Rausch, M. E. et al., “Obesity in C57BL/6J mice is characterized by adipose tissue hypoxia and cytotoxic T-cell infiltration,” Int J Obes (Lond) 32(3):451-63 (2008); and Kintscher, U. et al., “T-lymphocyte Infiltration in Visceral Adipose Tissue: A Primary Event in Adipose Tissue Inflammation and the Development of Obesity-Mediated Insulin Resistance,” Arterioscler Thromb Vase Biol 28(7):1304-10 (2008), the entire contents and disclosures of which are hereby incorporated by reference. The function of these fat-associated T-cells (T^{fat}) and their conceivable role in obesity and/or glucose homeostasis is unknown, and the antigen-specificity, activation history, and T-cell sublineage profiles of T^{fat} are also unknown. See, e.g., Wu, H. et al., “T-cell accumulation and regulated on activation, normal T cell expressed and secreted upregulation in adipose tissue in obesity,” Circulation 115:1029-1038 (2007), the entire contents and disclosure of which are hereby incorporated by reference.

An unsuspected immunoregulatory circuit has now been discovered in adipose tissue in which fat-resident T-cells (T^{fat}) appear to control the rate of weight gain, insulin resistance, and glucose metabolism in mice as well as humans. According to this model, a balance between pro-inflammatory IFNγ-secreting (Th1) T-cells and regulatory (or anti-inflammatory) Th2 and CD4+/Foxp3+ T-cells among T^{fat} generally exists in adipose tissue of normal weight individuals. However, in obese individuals, this critical balance between these T-cell compartments may become imbalanced leading to pro-inflammatory conditions and a reduction in insulin sensitivity in adipose tissue caused by an increase in the number of IFNγ-secreting (Th1) T-cells that eventually overwhelm the population of regulatory (or anti-inflammatory) Th2 and/or CD4+/Foxp3+ T-cells present in the tissue. Accordingly, it is proposed that immuno-modulatory compositions and methods may be used to restore the balance of T-cells in fat tissue, particularly visceral adipose tissue (VAT), which may be effective at restoring insulin sensitivity and combating progression of obesity, MetSyn, and/or T2D.

It is further proposed that treatments and therapies based on T-cell activation may be effective at modulating and restoring the balance between pro- and anti-inflammatory T-cells in adipose tissue. Activation of T-cells may normally be triggered by binding of T-cell receptors (TCRs) to presented non-self MHC-antigens, resulting in clonal proliferation and differentiation of the activated T-cell pool. This “activation” response of T-cells may be simulated by antibodies designed to bind a component(s) or subunit(s) of a T-cell receptor complex or an accessory molecule(s) present on the surface of a T-cell. Since these antibodies may target components of the TCR complex that are commonly present on T-cells, such simulated “activation” of T-cells by antibody binding may lead to broad polyclonal activation of the entire T-cell population, or at least an entire T-cell compartment. However, without co-stimulation by antigen-presenting cell (APC) molecules (e.g., CD28) and without a true MHC-bound antigen for binding to the TCR-complex, these T-cells “activated” by antibody binding undergo large scale activation-induced cell death (AICD) and anergy. Thus, despite exposure to a T cell “activating” antibody, the ultimate effect of such an antibody treatment is to broadly diminish or eliminate the total population of T-cells (or at least a T-cell compartment).

Such T cell “activating” antibodies targeting components of the TCR have been used or suggested as immuno-suppressants against various immune conditions or diseases including transplanted organ or tissue rejection, allergies, and autoimmunity and to improve tolerance. See, e.g., Wang, H. Y. et al., “Tumor-specific human CD4+ regulatory T cells and their ligands: implications for immunotherapy,” Immunity 20:107-118 (2004), the entire contents and disclosure of which is hereby incorporated by reference. Importantly, Fox3p+ regulatory T-cells appear to be relatively resistant to cell death induced by treatment with a particular T cell activating antibody against CD3. See, e.g., Chatenoud, L. et al., “CD3-specific antibodies: a portal to the treatment of autoimmunity,” Nat Rev Immunol 7:622-632 (2007), the entire contents and disclosure of which is incorporated by reference.

According to embodiments of the present invention, treatment with a T cell activating antibody that binds to an epitope present on a component(s) or a subunit(s) of the TCR complex, or to an epitope present on an accessory molecule of a T-cell, may be effective at modulating the immune environment within adipose tissue toward a more favorable proportion of anti-inflammatory T-cells, which may then improve insulin sensitivity in individuals, such as obese individuals, having (or at risk of developing) MetSyn and/or T2D. The use of immuno-suppressive or immuno-modulatory T-cell “activating antibodies” for treatment of obesity, MetSyn, and/or T2D has not been previously described or suggested. This is not surprising since the discovery of an immunological circuit involving T-cells present in adipose tissue is only presently described, which regulates obesity, glucose metabolism, insulin sensitivity, and glucose intolerance/toxicity in individuals, particularly in obese individuals, when this immunological circuit becomes imbalanced. Indeed, as shown in the examples below, treatment of mice with an anti-CD3 antibody or its F(ab′)₂ fragment, results in increased proportions of Fox3p+ T-cells, lowered fasting glucose and insulin levels, improved glucose tolerance and insulin sensitivity, and reduced hepatosteatosis, and treatment with the F(ab′)₂ fragment of anti-CD3 showed increased production of IL-10.

According to embodiments of the present invention, a composition comprising a T cell activating antibody may be administered to an individual, such as an overweight or obese individual, having or experiencing MetSyn and/or T2D, or at risk of developing MetSyn and/or T2D. For humans, an obese individual may be defined as a person or patient having a body mass index (BMI) in a range of about 25 to about 30, and an obese individual may be defined as a person or patient having a body mass index (BMI) of about 30 or greater. According to other embodiments, such an individual having, or at risk of developing, MetSyn and/or T2D may be an individual having a ratio of pro-inflammatory T-cells to regulatory (or anti-inflammatory) T-cells in adipose tissue, such as subcutaneous adipose tissue (SAT) or visceral adipose tissue (VAT), that is higher than such a ratio present in non-obese or normal weight individuals. Alternatively, an individual having, or at risk of developing, MetSyn and/or T2D may be an individual having a ratio of pro-inflammatory T-cells to regulatory (or anti-inflammatory) T-cells in adipose tissue, such as SAT or VAT, that is within a range of ratios of pro-inflammatory T-cells to regulatory (or anti-inflammatory) T-cells commonly observed in adipose tissue of individuals having MetSyn and/or T2D.

According to yet other embodiments, such an individual having, or at risk of developing, MetSyn and/or T2D may be an individual having MetSyn and/or T2D according to standard clinical and/or pathological criteria, which may be based on known symptoms or pathological signs of MetSyn and/or T2D. Such an individual may be an individual having any one or more of the following: elevated glucose levels, hyperinsulinemia, glucose intolerance, insulin resistance, obesity, and hepatic steatosis. For example, an individual having, or at risk of developing, MetSyn and/or T2D may be a human individual having MetSyn or prediabetes and/or at risk of developing T2D. A human individual may have MetSyn or prediabetes and/or be at risk of developing T2D (1) if such an individual has a fasting or preprandial blood glucose level in a range of about 5.5 to about 7.0 mmol per liter (i.e., about 100 to about 125 mg per liter), or (2) if such an individual has a blood glucose level in a range of about 7.8 to about 11.1 mmol per liter (i.e., about 140 to about 200 mg per liter) in an oral glucose tolerance test (OGTT) two hours after ingesting a 75-gram glucose drink. Alternatively, for example, an individual having, or at risk of developing, MetSyn and/or T2D may be a human individual having T2D. A human individual may have T2D (1) if such an individual has a fasting or preprandial blood glucose level of about 7.0 mmol per liter or greater (i.e., about 125 mg per liter or greater), or (2) if such an individual has a blood glucose level of about 11.0 mmol per liter or greater (i.e., about 200 mg per liter or greater) in an oral glucose tolerance test (OGTT) two hours after ingesting a 75-gram glucose drink. Alternatively, according to some embodiments, such an individual having, or at risk of developing, MetSyn and/or T2D may be an individual having known mutations and/or genetic risk factors associated with increased likelihood of developing MetSyn and/or T2D.

According to embodiments of the present invention, a T cell activating antibody may comprise any antibody that binds to an epitope present on a component(s) or subunit(s) of a T-cell receptor complex, such as a TCRα or TCRβ chain of the TCR, or an accessory molecule(s) present on the surface of a T-cell. Such a T cell activating antibody may include any type of immunoglobulin or immunoglobulin-like polypeptide(s), or portion(s) or fragment(s) thereof, including, for example, a monoclonal, polyclonal, purified, recombinant, mutagenic, chemically modified, etc., antibody or immunoglobulin polypeptide, or a fragment(s) or portion(s) thereof.

According to some embodiments, a T cell activating antibody may be generated, for example, by immunizing an animal with an immunogenic amount of an antigen, such as a component or subunit of a T-cell receptor complex or an accessory molecule of a T-cell, or a portion thereof, which may be emulsified in an adjuvant, such as Freund's complete adjuvant, and/or administered over a period of weeks in intervals that may range from about two to about six weeks. Such methods may include, for example, a first immunization in Freund's complete adjuvant and subsequent immunizations in Freund's incomplete adjuvant (at biweekly to monthly intervals thereafter). Depending on whether monoclonal or polyclonal antibodies are desired, B lymphocyte cells, such as spleen cells, may be extracted from immunized animals and fused with myeloma cells, or serum may be isolated from immunized animals, respectively. Test bleeds may also be taken at regular intervals, such as at fourteen day intervals between a second and a third immunization with production bleeds taken at monthly intervals thereafter.

According to some embodiments, a T cell activating antibody may include a monoclonal antibody (mAb). Methods for generating monoclonal antibodies are known in the art. In general, antibody-producing B lymphoid cells collected from an animal that has been injected with an immunogen or antigen may be fused in culture with immortalized myeloma cells having mutations in certain genes that may be used as a basis for selection to form hybridoma cells that continue to produce antibodies. For example, spleen cells may be harvested from an immunized animal and mixed with a myeloma cell line with B lymphocyte and myeloma cells induced to fuse by addition of polyethylene glycol.

For selection purposes, the myeloma cells may contain mutations in the thymidine kinase (TK) and hypoxanthine guanine phosphoribosyl transferase (HGPRT) genes. Normally, animal cells synthesize purine nucleotides and thymidylate de novo from phosphoribosyl pyrophosphate and uridylate, respectively, which are required for DNA synthesis. However, anti-folate drugs, such as aminopterin, may block this de novo pathway, thus forcing the cells to utilize a salvage pathway to synthesize purines and thymidylate from exogenously supplied hypoxanthine and thymidine. Therefore, cells grown in the presence of hypoxanthine, aminopterin, and thymidine (i.e., HAT medium) are able to grow in the presence of aminopterin using the salvage pathway. On the other hand, myeloma cells having mutations in the TK and HGPRT genes are unable to grow under such conditions because of the unavailability of the salvage pathway. Therefore, only mutant myeloma cells that have fused with normal B cells to form hybridoma cells are complemented with normal TK and HGPRT genes allowing them to grow in HAT medium containing aminopterin. B cells that do not become fused with myeloma cells do not survive in culture because they are not immortalized.

Hybridoma technology permits one to explore the entire antibody producing B lymphocyte repertoire of the immune system and to select those cells that produce specific antibodies having the desired binding affinity to a specific antigen, such as a component(s) or subunit(s) of a T-cell receptor complex or an accessory molecule(s) present on the surface of a T-cell. In producing monoclonal antibodies, mutant myeloma cells are fused with a population of B cells extracted from an immunized animal with each B cell, and hence each hybridoma cell, producing a pool of antibodies directed against a single epitope of an antigen. In other words, each fusion event produces a clonal population of hybridoma cells that may be maintained in culture and used to produce a homogeneous pool of antibodies having affinity for a specific epitope. Only those hybridoma cell lines that produce antibodies having a desired affinity for the specific antigen of interest may then be selected or screened from the repertoire of antibody-producing hybridoma cells using any known method in the art (e.g., Western blotting, ELISA, etc.). Thus, monoclonal antibodies provide a pool of identical antibodies directed against a specific epitope of a single antigen. As a result, according to some embodiments, a monoclonal T cell activating antibody may have a therapeutic advantage over a polyclonal antibody by minimizing side effects and non-specific binding of the T cell activating antibody to molecules other than the desired component(s) or subunit(s) of a T-cell receptor complex or accessory molecule(s) present on the surface of a T-cell.

According to some embodiments, a T cell activating antibody may be a polyclonal antibody. Methods for generating polyclonal antibodies are known in the art. Generally speaking, serum is removed from an animal immunized with a specific immunogen or antigen, such as a component or subunit of a T-cell receptor complex or an accessory molecule of a T-cell, or a portion thereof. Such serum will contain antibodies against multiple epitopes and antigens and may further contain antibodies specific for the antigen of interest, such as a component(s) or subunit(s) of a T-cell receptor complex or an accessory molecule(s) present on the surface of a T-cell. Therefore, since a polyclonal antiserum is taken from whole animal blood, such polyclonal antiserum may contain many different antibodies that are capable of binding to a wide diversity of epitopes and antigens, many of which may be unrelated to the antigen of interest. Polyclonal antisera may also contain multiple antibodies that recognize and bind to distinct epitopes of the same antigen (including the antigen of interest) with varying affinity and avidity. To increase their usefulness, such polyclonal antisera may be purified prior to their use by selecting antibodies that bind to the antigen of interest. For example, polyclonal antibodies that recognize and bind to the antigen of interest may be selected by affinity chromatography or purification using at least a portion of the antigen as bait.

According to some embodiments of the present invention, a T cell activating antibody may include an IgA, IgD, IgE, or IgM antibody isotype, as well as subclasses thereof. However, IgG antibodies are used therapeutically more often than the other isotypes. In addition, such a T cell activating antibody may include a functional antibody fragment or engineered version of any of these antibody isotypes. Methods for preparing and modifying antibodies and immunoglobulin-like polypeptides that may be used according to some embodiments of the present invention are generally known in the art. According to some embodiments, a T cell activating antibody may include any antibody, or a functional fragment or an engineered version thereof, which has been shown to bind with specificity to an antigen of interest, may be further engineered to optimize its binding characteristics and/or design for expression by any known chemical, enzymatic, recombinant, etc., technique.

The basic structure of an IgG antibody is a symmetrical tetrameric Y-shaped complex consisting of two identical light polypeptide chains and two identical heavy polypeptide chains. The heavy chains are linked to one another through at least one disulfide bond, and each light chain is linked to a contiguous heavy chain by a disulfide linkage. Both heavy and light chains may be divided into a series of homologous immunoglobulin (Ig) domains of about 110 amino acids. Each of the Ig domains of the heavy and light chains may be divided into variable (V) or constant (C) regions or domains. In both heavy and light chains, the most N-terminal Ig domain is a variable domain, whereas the remaining Ig domains comprise constant domains. Two antigen-binding sites are formed at the N-terminal ends of each arm of the IgG antibody with the N-terminal ends of each arm comprising the N-terminal variable domains of the heavy and light chains. Within each variable region or domain of the heavy and light chains, there are three hypervariable or complementarity-determining regions (CDRs) that are surrounded by relatively more conserved framework regions. These CDR regions are responsible for much of the amino acid sequence variation between antibodies produced by different cells, which are largely responsible for differences in specificity and affinity to distinct epitopes and antigens.

According to some embodiments, a T cell activating antibody may include an antibody fragment. One method for generating antibody fragments is to subject an antibody of interest to limited proteolytic cleavage/digestion and/or chemical treatment. For example, the proteolytic enzyme, papain, preferentially cleaves the IgG antibody on the N-terminal side of the hinge region to produce three fragments, including two identical Fab (fragment, antigen-binding) fragments and one Fc (fragment, crystalline) fragment. On the other hand, the proteolytic enzyme, pepsin, preferentially cleaves the IgG antibody on the C-terminal side of the hinge region to produce one stable fragment called F(ab′)₂ (two Fab′ fragments held together by an intact hinge region and disulfide bond). The remaining constant regions of the antibody are degraded. Both Fab and F(ab′)₂ fragments retain the antigen-binding variable regions. Fab fragments each have a single antigen-binding site, whereas F(ab′)₂ fragments have two antigen-binding sites. Because Fab and F(ab′)₂ fragments lack all or a portion of the Fc region, a T cell activating antibody comprising a Fab and F(ab′)₂ fragment according to embodiments of the present invention may result in fewer side effects and complications that might otherwise occur with whole antibodies via Fc-mediated non-specific stimulation of the immune system through interactions with other immune cells.

According to some embodiments, a T cell activating antibody may include a Fab or F(ab′)₂ fragment of a T cell activating antibody that binds to an epitope present on a component(s) or subunit(s) of a T-cell receptor complex or an accessory molecule(s) present on the surface of a T-cell. Such a Fab or F(ab′)₂ fragment of a T cell activating antibody may have a therapeutic benefit over the whole antibody of being less mitogenic (i.e., the Fab or F(ab′)₂ fragments may cause less proliferation of T-cells and lower production of cytokines compared to the whole antibody), thus resulting in fewer complications following treatment.

According to some embodiments, such a T cell activating antibody may be an antibody or immunoglobulin polypeptide that binds to an epitope present on one or more subunits of CD3, or a functional fragment or engineered version thereof, such as a Fab and F(ab′)₂ fragment of such an antibody. For example, such a T cell activating antibody may include an antibody that binds to any subunit of CD3, such as an anti-CD3 antibody, or a functional fragment or engineered version thereof, which binds to the δ, ε, and/or γ subunits of CD3.

According to some embodiments, such a T cell activating antibody may be an antibody or immunoglobulin polypeptide that binds to an epitope present on one or more subunits of CD4 or CD8, which are each an accessory molecule that may associate with the TCR complex, or a functional fragment or engineered version thereof, such as a Fab and F(ab′)₂ fragment of such an antibody. According to other embodiments, such a T cell activating antibody may be an antibody or immunoglobulin polypeptide that binds to an epitope present on other accessory molecules, or a functional fragment or engineered version of such an antibody or immunoglobulin polypeptide, such as a Fab and F(ab′)₂ fragment.

According to some embodiments of the present invention, a T cell activating antibody may include an antibody currently in clinical use or currently undergoing preclinical or clinical trials in any country. The T cell activating antibody may include, for example, “Parenteral 145-2C11” or its F(ab′)₂ fragment, “Parenteral 04.18”, Visilizumab (NUVION™; PDL BioPharma, Inc.), “OKT3” (Ortho Biotech), “hOKT3γ1 Ala-Ala” (Teplizimab), or ChAglyCD3.

According to some embodiments, a T cell activating antibody may comprise either a FcR-binding antibody or a FcR-non-binding antibody. A FcR-non-binding antibody may be made, for example, by mutating key amino acids in the Fc region necessary for interaction with a Fc receptor present on other immune cells. In contrast to FcR-non-binding antibodies, FcR-binding antibodies are generally more potent mitogens that elicit release of toxic levels of T-cell derived cytokines that can cause complications in patients. Therefore, a T cell activating antibody comprising a FcR-non-binding antibody may have therapeutic advantages over a FcR-binding antibody. See, e.g., Chatenoud, L. et al., supra (2007); and Herold K. C. et al., “Activation of human T-cells by FcR nonbinding anti-CD3 mAb, hOKT3gamma1(Ala-Ala),” J Clin Invest 111:409-418 (2003), the entire contents and disclosures of which are hereby incorporated by reference.

According to some embodiments of the present invention, a T cell activating antibody may include an antibody or immunoglobulin-like polypeptide produced by any known and available recombinant techniques. In addition, clones encoding an antibody or immunoglobulin-like polypeptide shown to bind an antigen of interest, such as a component(s) or subunit(s) of a T-cell receptor complex or an accessory molecule(s) of a T-cell, or a portion thereof, may be further subjected to mutagenesis and selection for antibody evolution in vitro to improve affinity for the antigen of interest. See, e.g., He et al., “Ribosome display: next generation display technologies for production of antibodies in vitro,” Expert Rev Proteomics 2(3): 421-30 (2005); Wark et al., “Latest technologies for the enhancement of antibody maturity,” Adv Drug Deliv Rev 58(5-6): 657-70 (2006); and U.S. patent application Ser. No. 12/026,412, the entire contents and disclosure of which are hereby incorporated by reference. Recombinant methods for engineering and synthesizing antibodies or immunoglobulin-like polypeptides may provide a more stable genetic source compared to hybridoma cell lines and may also be produced more quickly and economically using, for example, bacterial expression systems.

According to some embodiments, once a monoclonal antibody or immunoglobulin polypeptide expressed from a library has been identified as having a desired affinity for an antigen of interest, such as a component(s) or subunit(s) of a T-cell receptor complex or an accessory molecule(s) of a T-cell, or a portion thereof, such an antibody may be engineered and designed for expression on the basis of its known sequence and used as a T cell activating antibody. For example, a cDNA may be generated from mRNA isolated from hybridoma cells that produce the antibody showing the desired specificity and affinity for particular antigen. Once the cDNA sequence is cloned into a vector, it may be manipulated and engineered as desired by standard recombinant techniques. For example, the sequence may be truncated to include only a functional portion of a full heavy and/or light chain antibody sequence, linker sequences may be added to connect functional fragments, chimeric antibodies having portions from different species may be created, etc. The resulting sequence may then be screened for binding of the encoded antibody or immunoglobulin polypeptide to the antigen. The smallest antibody fragment that retains antigen binding is a Fv fragment (variable domain fragment; i.e., a heterodimer of heavy and light chain variable regions). However, Fv fragments are not easily expressed in bacteria and may dissociate without chemical cross-linking.

According to some embodiments, a T cell activating antibody of the present invention may include an alternative form of a Fv fragment, such as a single chain Fv (scFv) that contains V_{L} and V_{H} domains joined by a linker peptide sequence that are transcribed together as a single transcript to form a single polypeptide chain that binds to an antigen, such as a component(s) or subunit(s) of a T-cell receptor complex or an accessory molecule(s) of a T-cell, or a portion thereof. Depending on the length and amino acid composition of the linker sequence as well as the number of V_{L} and V_{H} domains joined by linker sequences, then scFv fragments may be either monovalent or multivalent (i.e., referring to the number of antigen-binding sites) between one or more interacting scFv fragments.

According to some embodiments, any T cell activating antibody may be synthesized or expressed by any known or alternative method. For example, Fab and F(ab′)₂ fragments may be generated by recombinant techniques instead of by limited digestion. According to some embodiments of the present invention, any expression system known in the art may be used to express an antibody or immunoglobulin-like polypeptides, or functional fragment thereof, such as in bacteria, yeast, plants, cultured animal cells, etc. See, e.g., Borrebaeck, C., “Antibody engineering,” Breakthroughs in Molecular Biology, (Second Edition, Oxford University Press, Oxford, UK, 1995), the entire contents and disclosure of which are hereby incorporated by reference. For example, when E. coli cells are used for expression, antibodies or immunoglobulin-like polypeptides, or fragments thereof, may be expressed with an appropriate leader or signal peptide sequence, extracted from the periplasmic space, and purified according to standard methods, such as affinity purification using antigen, protein A, protein G, etc. See, e.g., Zola, H., “Monoclonal Antibodies,” The Basics: from background to bench, Kingston, F. (Ed.), (BIOS Scientific Publishers Limited, Oxford, 2000), the entire contents and disclosure of which are hereby incorporated by reference. Alternatively, antibodies or immunoglobulin polypeptides, or fragments thereof, may be expressed and secreted into the surrounding medium using an appropriate vector and bacterial strain. See, e.g., Hoogenboom et al., “Multi-subunit proteins on the surface of filamentous phage: methodologies for displaying antibody (Fab) heavy and light chains,” Nucleic Acids Res. 19(15):4133-37 (1991). However, entry of the antibody or immunoglobulin like polypeptide chains into the oxidizing environment of the bacterial periplasm may be required for proper folding and formation of disulfide bonds.

According to some embodiments of the present invention, T cell activating antibodies may include antibodies or immunoglobulin-like polypeptides that are identified by screening a library of antibody sequences or a collection of cDNAs for binding to an antigen of interest, such as a component(s) or subunit(s) of a T-cell receptor complex or an accessory molecule(s) present of a T-cell, or a portion thereof. Such screening techniques may include any method for identifying clones that encode antibodies or immunoglobulin-like polypeptides that bind with specificity and affinity to the antigen of interest, including, for example, phage display, ribosomal display, bacterial display, yeast display, etc. or related techniques. See, e.g., Mondon et al., “Human antibody libraries: a race to engineer and explore a larger diversity,” Front Biosci 13: 1117-1129 (2008); Lowman et al., “Selecting High-Affinity Binding Proteins by Monovalent Phage Display,” Biochemistry 30(45):10832-10838 (1991); Clackson et al., “Making antibody fragments using phage display libraries,” Nature 352(6336):624-628 (1991); and Cwirla et al., “Peptides on phage: a vast library of peptides for identifying ligands,” PNAS USA 87(16):6378-6382 (1990), the entire contents and disclosures of which are hereby incorporated by reference.

Phage display methods are generally based on producing genetically altered phage particles, such as recombinant M13 or Fd phages, that display a recombinant protein containing a particular antibody or engineered immunoglobulin-like polypeptides on the surface of the phage particle by fusion with a phage coat protein. Such recombinant phages may be grown and isolated using known methods. By screening individual phage particles for their binding to an antigen of interest, antibodies or engineered immunoglobulin-like polypeptides displayed on the surface of such phage particles may be identified and their sequences readily determined and cloned.

The following provides an exemplary procedure for screening a library of sequences encoding antibodies and immunoglobulin-like polypeptides, and fragments thereof, for binding to an antigen of interest, such as a component(s) or subunit(s) of a T-cell receptor complex or an accessory molecule(s) of a T-cell, or a portion thereof, using a phage display method. For example, candidate sequences may first be made by standard reverse transcriptase protocols to generate cDNA molecules from mRNA isolated from a hybridoma that produces a monoclonal antibody known to bind to an antigen of interest. Such cDNA may then be inserted into a vector and engineered to have desired binding and expression characteristics. cDNA may then be sub-cloned into a plasmid. For example, such cDNA sequence may be engineered into a scFv sequence for expression in vitro. To make a scFv, the cDNA molecules encoding the variable regions of the heavy and light chains of the monoclonal antibody may be amplified by standard polymerase chain reaction (PCR) methods using a set of degenerate primers for framework regions of mouse immunoglobulin heavy and light variable regions. Amplified heavy and light chain variable regions may then be linked together with at least one linker oligonucleotide in order to generate a recombinant scFv DNA molecule.

To screen sequences for their ability to bind the antigen of interest, each scFv DNA sequence may be ligated into a filamentous phage plasmid designed to fuse the amplified scFv sequences into the 5′ region of the phage gene encoding a phage coat protein. E. coli cells may then be transformed with the recombinant phage plasmids, and filamentous phage grown and harvested. The desired recombinant phages display antigen-binding domains fused to the amino terminal region of the minor coat protein. Such “display phages” may then be passed over immobilized antigen, for example, using the method known as “panning” to adsorb those phage particles containing scFv antibody proteins that are capable of binding antigen. The antigen-binding phage particles may then be amplified by standard phage infection methods, and the amplified recombinant phage population again selected for antigen-binding ability. Such successive rounds of selection for antigen-binding ability, followed by amplification, select for enhanced antigen-binding affinity among the ScFvs displayed on recombinant phages.

Selection for increased antigen-binding affinity may be made by adjusting the conditions under which binding takes place to require a higher binding affinity. As mentioned above, enhanced antigen-binding affinity may also be achieved by altering nucleotide sequences of the DNA sequence encoding, for example, the variable antigen-binding domain of the scFv and then subjecting recombinant phage populations to successive rounds of selection for antigen-binding affinity and amplification.

In addition to restoring a balance between pro-inflammatory T-cells and regulatory cells in adipose tissue, treatment with anti-CD3 antibody appears to further cause both CD4+ and CD8+ T-cells to secrete leukemia inhibitory factor (LIF) systemically and in both SAT and VAT. In support of LIF playing a role in glucose metabolism perhaps downstream of T-cell activation by anti-CD3, i.p. injection of LIF results in lowering of glucose levels in a dose dependent manner, and injection of a neutralizing anti-LIF antibody reduced glucose tolerance. Therefore, according to some embodiments of the present invention, a composition comprising a LIF protein or peptide, or a functional portion or fragment thereof, or an analog molecule that ligates a LIF receptor, may be administered to an individual, such as an overweight or obese individual, experiencing or having MetSyn and/or T2D, or at risk of developing MetSyn and/or T2D, to alleviate the condition, syndrome, or disease. According to some embodiments, a composition comprising a functional portion or fragment of a LIF protein may be any portion or fragment of a LIF protein that retains one or more biological activities of functions of the full-length LIF protein, such as the ability to bind a cytokine receptor (e.g., a LIF receptor or LIF receptor/gp130 heterodimer) in vivo.

According to other embodiments, a composition comprising a LIF protein, or a functional portion or fragment thereof, or an analog molecule that ligates a LIF receptor, may be administered to an individual, such as an overweight or obese individual, experiencing or having MetSyn and/or T2D, or at risk of developing MetSyn and/or T2D, in combination with a T cell activating antibody to achieve a combined therapeutic effect. According to these embodiments, the T cell activating antibody and the LIF protein, or functional portion or fragment thereof, or the analog molecule that ligates a LIF receptor, may either be formulated together as parts of the same composition or formulated separately in different compositions that are administered in tandem.

According to other embodiments of the present invention, a composition comprising a protein having a high degree of homology or identity to a known LIF protein sequence, or a functional portion or fragment thereof, may be administered to an individual, such as an overweight or obese individual, experiencing or having MetSyn and/or T2D, or at risk of developing MetSyn and/or T2D, to alleviate the condition, syndrome, or disease. Such a protein having a high degree of homology or identity to a known LIF protein sequence may be at least about 80% similar or identical to a known LIF protein sequence, or alternatively, at least about 90% similar or identical to a known LIF protein sequence.

The sequence of leukemia inhibitory factor (LIF) from a variety of different organisms is known in the art. For example, the known full-length protein sequence of human LIF is as follows (SEQ ID NO. 1):

MKVLAAGVVPLLLVLHWKHGAGSPLPITPVNATCAIRHPCHNNLMNQIRS
QLAQLNGSANALFILYYTAQGEPFPNNLDKLCGPNVTDFPPFHANGTEKA
KLVELYRIVVYLGTSLGNITRDQKILNPSALSLHSKLNATADILRGLLSN
VLCRLCSKYHVGHVDVTYGPDTSGKDVFQKKKLGCQLLGKYKQIIAVLAQ
AF

According to embodiments of the present invention, a therapeutically effective amount of a T cell activating antibody or a LIF protein, or a functional fragment or analog thereof, may be administered individually or in combination to an individual, such as an overweight or obese individual, experiencing or having MetSyn and/or T2D or at risk of developing MetSyn and/or T2D. According to some embodiments, such a “therapeutically effective amount” may be an amount of a T cell activating antibody or a LIF protein, or a functional fragment thereof, effective to reduce or improve any known clinical symptoms or pathological signs associated with MetSyn and/or T2D, which may include any one or more of the following: elevated fasting or non-fasting glucose levels, fasting or non-fasting hyperinsulinemia, glucose intolerance, insulin resistance, obesity, and hepatic steatosis. According to some embodiments, such a “therapeutically effective amount” of a T cell activating antibody may be an amount effective to at least transiently reduce the number of T-cells in circulation, particularly the total number of pro-inflammatory T-cells, present in the body of an individual having MetSyn and/or T2D or at risk of developing MetSyn and/or T2D. Such a reduction in the total number of T-cells may be transient since the total number of T-cells may recover once treatment with the therapeutically effective amount of the T cell activating antibody is discontinued. According to some embodiments, such a “therapeutically effective amount” of a T cell activating antibody may be an amount effective to transiently reduce the number of circulating T-cells present in the body of the individual by about 30% or greater.

According to some embodiments, such a “therapeutically effective amount” of a T cell activating antibody or a LIF protein, or a functional fragment thereof, may be an amount effective to reduce fasting or non-fasting glucose and/or insulin levels present in such an individual. According to other embodiments, such a “therapeutically effective amount” of a T cell activating antibody or a LIF protein, or a functional fragment thereof, may be an amount effective to improve insulin sensitivity and/or glucose tolerance in peripheral tissues, such as VAT or SAT, of such an individual. According to other embodiments, such a “therapeutically effective amount” of a T cell activating antibody or a LIF protein, or a functional fragment thereof, may be an amount effective to reduce the body weight or fat mass of such an individual and/or to reduce the amount of hepatic steatosis.

According to some embodiments for compositions and methods of the present invention, a therapeutically effective and acceptable dosage range for an anti-CD3 antibody in humans may be in a range from about 0.1 to about 1.0 mg of the anti-CD3 antibody per kg of body weight. According to some embodiments for compositions and methods of the present invention, a therapeutically effective and acceptable dosage range in humans for a Fab or F(ab′)₂ fragment of an anti-CD3 antibody may be in a range from about 0.1 to about 1.0 mg of the Fab or F(ab′)₂ fragment of the anti-CD3 antibody per kg of body weight. According to some embodiments for compositions and methods of the present invention, a therapeutically effective and acceptable dosage range in humans for a LIF peptide or protein, or a functional fragment thereof, may be in a range from about 0.1 to about 1.0 mg of the LIF peptide, or the functional fragment thereof, per kg of body weight. These dosage ranges may be extrapolated from observations in obese C57/BL6J mice.

According to some embodiments of the present invention, a pharmaceutical composition may be administered to an individual experiencing or having MetSyn and/or T2D, or at risk of developing MetSyn and/or T2D. Such pharmaceutical composition may comprise a therapeutically effective amount of a T cell activating antibody and/or a LIF protein, or a functional portion thereof, in combination with a pharmaceutically acceptable carrier. Examples of pharmaceutically acceptable carriers and other suitable additives and adjuvants for pharmaceutical compositions that may be used according to some embodiments in combination with a T cell activating antibody and/or a LIF protein, or a functional portion thereof, may include any carrier, adjuvant, additive, etc., known to those skilled in the pharmacological art.

For pharmaceutical compositions administered according to embodiments of the present invention, a pharmaceutically acceptable carrier may be either liquid or solid and may include solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, fillers, diluents, disintegration agents, lubricants, sweetening agents, flavoring agents, dyes, glidants, wetting agents, etc., and any combinations thereof. Since a pharmaceutical composition administered to an individual according to embodiments of the present invention will generally comprise a T cell activating antibody or a LIF protein, such a pharmaceutical composition will generally be administered in the form of a liquid via a parenteral route of administration. However, even though a pharmaceutical composition may generally be administered parenterally as a liquid according to embodiments of the present invention, it is contemplated that a pharmaceutical composition administered to an individual according to embodiments of the present invention may be initially manufactured or provided in the form of a solid, such as a crystals or powders which may be dry, damp, wet, etc., and such a pharmaceutical composition may only subsequently be placed or dissolved into an appropriate carrier liquid or solution prior to administration to such an individual.

For a description of pharmaceutical compositions, carriers, etc. that may be used in formulating a pharmaceutically appropriate or acceptable composition administered according to embodiments of the present invention, see, for example, Remington, Pharmaceutical Science, (17th ed., Mack Publishing Company, Easton, Pa., 1985); Goodman & Gillman, The Pharmacological Basis of Therapeutics, (11^{th} Edition, McGraw-Hill Professional, 2005); and Griffin P. et al., The Textbook of Pharmaceutical Medicine, (Blackwell Publishing, Malden, Mass., 2006), the contents and disclosures of which are hereby incorporated by reference. See also, for example, U.S. Pat. Nos. 7,495,085; 7,495,084; 7,491,391; 7,449,555; and 7,488,806, the contents and disclosures of which are hereby incorporated by reference. Except insofar as any conventional pharmaceutical carrier is incompatible with pharmaceutical compositions administered according to embodiments of the present invention, their potential use in such pharmaceutical compositions is contemplated.

The mode or route of administration according to embodiments of the present invention may be selected to maximize delivery to a desired target site in the body of an individual, subject, or patient. According to embodiments of the present invention, pharmaceutical compositions may generally be administered via a parenteral route. Suitable parenteral routes may include intravascular (such as bolus or infusion intravenous), intraperitoneal, intramuscular, intradermal, subcutaneous, etc., routes. Pharmaceutical compositions administered according to embodiments of the present invention may be administered in a variety of unit dosage forms depending on the method of administration. For parenteral administration, pharmaceutical compositions administered according to some embodiments of the present invention may be formulated as sterile solutions, emulsions, suspensions, etc. as known and/or used in the art.

Pharmaceutical compositions of the present invention may be administered according to embodiments of the present invention either as a single dose or as part of a dosage regimen. A dosage regimen may be adjusted to provide an optimum therapeutic response. For example, several different doses may be administered daily or doses may be proportionally reduced as indicated by the exigencies of the therapeutic situation. A pharmaceutical composition may be administered as part of a dosage regimen and circulating concentrations of a T cell activating antibody or a LIF protein, or a functional fragment thereof, may be allowed to reach a desired equilibrium concentration through a series of doses. For convenience, a predetermined total daily dosage of a pharmaceutical composition may be divided and administered in portions during the day as required. A pharmaceutical composition may be administered according to a dosage regimen including from about 1 to about 5 doses per day. For example, 1, 2 or 3 doses may be administered per day for a limited number of days, such as one injection per day for about 5 days.

According to another broad aspect of the present invention, it is contemplated that autoantigen molecules present in adipose tissue, such as in visceral adipose tissue (VAT), may be used as a basis for the development of a vaccine for the treatment, prevention, inhibition, amelioration, reversal, etc. of obesity, MetSyn, and/or T2D. As described herein, local rearrangements of the TCRα locus occur in adipose tissue of DIO mice using a B6 “OT2” TCR transgenic mouse model. Without these secondary rearrangements, OVA-specific T-cells transferred into RAG^{null} mice are unable to improve glucose tolerance, insulin sensitivity, fasting blood glucose, fasting insulin levels, or body weight suggesting that these local rearrangements of the TCRα locus in adipose tissue are necessary to affect glucose metabolism in this tissue (i.e., these metabolic T cells and their effects are antigen-driven and antigen-specific). In addition, as described in greater detail below, strong evidence from the observed TCR bias suggests that there is a very narrow (and perhaps singular) spectrum of auto-antigenic epitope(s) that may drive the abnormal pro-inflammatory response with progressive insulin resistance and glucose intolerance in obese individuals with MetSyn and/or T2D. Therefore, it is contemplated that tolerogenic vaccination methods (immunotherapy) for reducing or eliminating this auto-immunity in individuals with MetSyn and/or T2D may be used to treat, inhibit, prevent, ameliorate, reverse, etc. progression and development of these metabolic conditions or diseases.

According to these embodiments of the present invention, an identified auto-antigen, or a fragment thereof, that is responsible for helping to trigger the auto-immune response in adipose tissue of obese individuals having, or at risk of developing, MetSyn and/or T2D may be administered (e.g., injected) into such individuals to promote tolerance toward such antigens present in the body. A suggested mechanism for this tolerogenic vaccination approach is that the injected antigen circulates in the body and binds to TCRs present on the surface of cognate T-cells, which may be responsible for triggering an autoimmune condition or disease. However, there is no co-stimulation provided via accessory proteins because the antigen is not presented as a MHC fusion on the surface of an antigen-presenting cell (APC). Therefore, this T-cell pool that might otherwise trigger the autoimmune condition or disease becomes reduced or eliminated via AICD and/or anergy. Furthermore, a combination of such an immunotherapy with a relatively low dose treatment series of a T cell activating antibody, such as an anti-CD3 antibody or a F(ab′)₂ fragment of an anti-CD3 antibody, may increase the tolerogenic character of such an immunotherapy.

### EXAMPLES

C57BL/6 (B6) mice have the propensity to develop diet induced obesity (DIO). See, e.g., Parekh, P. I. et al., “Reversal of diet-induced obesity and diabetes in C57BL16J mice,” Metabolism 47:1089-1096 (1998); Petro, A. E. et al., “Fat, carbohydrate, and calones m the development of diabetes and obesity in the C57BL16J mouse,” Metabolism 53:454-457 (2004); and Rossmeisl, M. et al., “Variation in type 2 diabetes-related traits in mouse strains susceptible to diet-induced obesity,” Diabetes 52:1958-1966 (2003), the entire contents and disclosures of which are hereby incorporated by reference. The impact of T-cells on glucose homeostasis in B6 mice with diet-induced obesity (DIO) is investigated as described below.

A strong bias in the recruitment of pro-inflammatory Th1, but not Th17, T-cells into adipose tissue of B6 DIO mice is observed. Such bias in the recruitment of pro-inflammatory Th1 increases in a body mass-dependent manner, while much smaller amounts of CD4+Foxp3+ anti-inflammatory T-cells are found in adipose tissue of obese mice and humans. In T-cell receptor transgenic studies, visceral fat T cells selectively undergo secondary, highly biased TCRα rearrangements, indicative of a cognate selection process reminiscent of organ selective autoimmunity. See, e.g., Amrani, A. et al., “Progression of autoimmune diabetes driven by avidity maturation of a T-cell population,” Nature 406:739-742 (2000), the entire contents and disclosure of which are hereby incorporated by reference. The predominant T-cell effect in glucose homeostasis, as revealed by T-cell reconstitution studies in lymphocyte-free DIO RAG^{null} mice appears to be improvement of glucose tolerance, enhanced insulin-sensitivity, and lessening of weight gain. This T-cell effect is probed with an immunotherapy utilizing a clinically effective αCD3 antibody and a non-mitogenic F(ab′)₂ fragment of the αCD3 antibody. Both αCD3 antibody and F(ab′)₂ antibody fragment therapy are shown to dramatically normalize glucose homeostasis and to selectively restore CD4+Foxp3+ T-cell pools in VAT of DIO mice. Such treatment is effective for greater than 4 months after a single course of injections despite continued high-fat diet consumption of these mice. Thus, anti-inflammatory cells of T^{fat} may provide a physiological mechanism to counter-regulate inflammation-induced insulin resistance, and anti-inflammatory mechanisms may be exploited therapeutically to treat individuals with MetSyn and/or T2D.

### Example 1

### Distribution and Sublineage Profile of T^{fat}

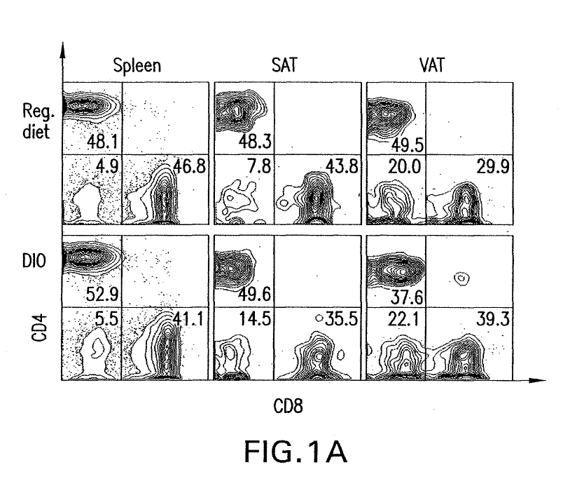

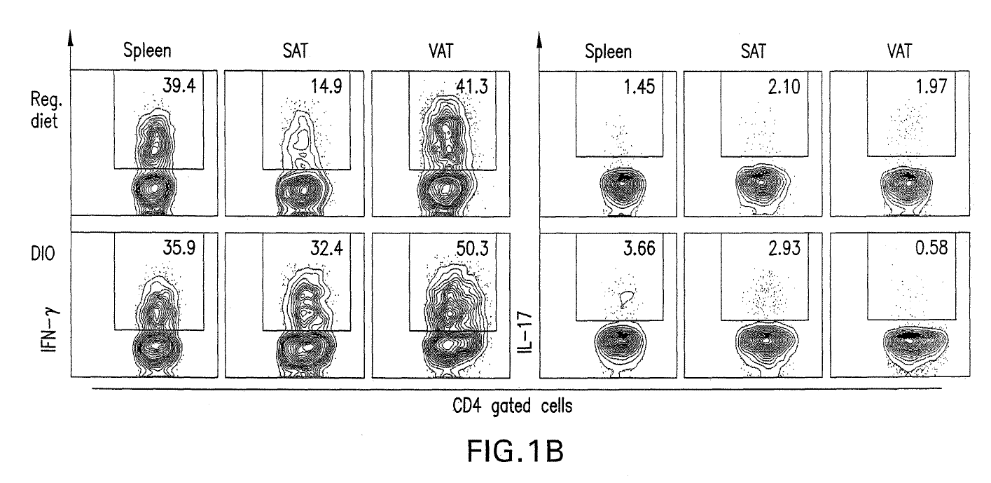

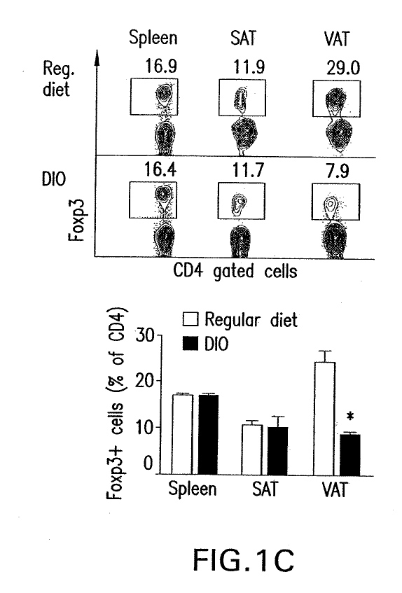

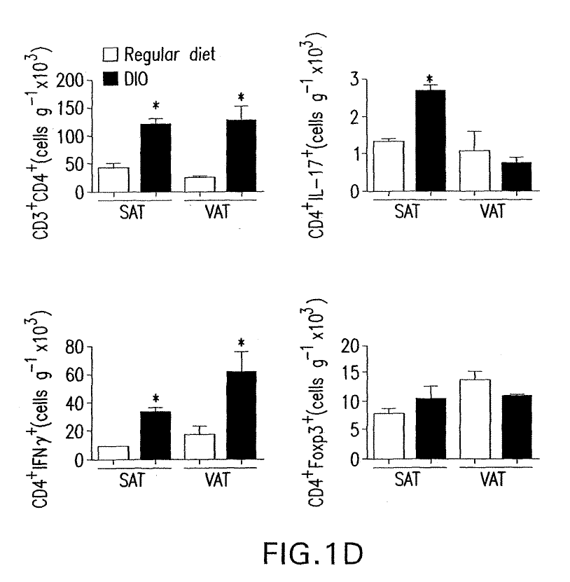

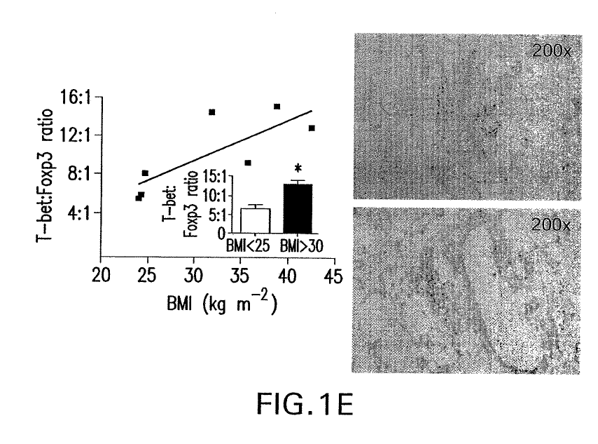

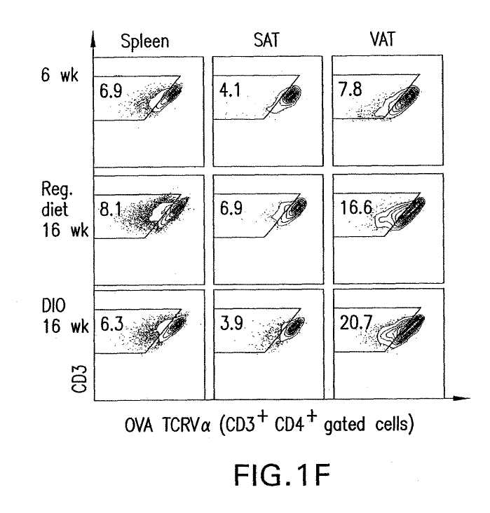

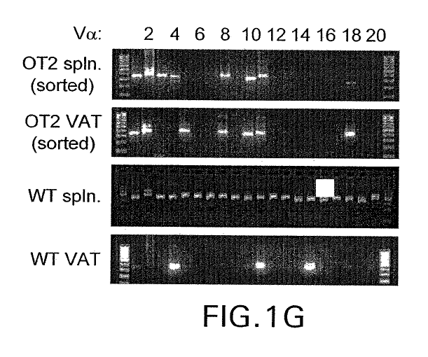

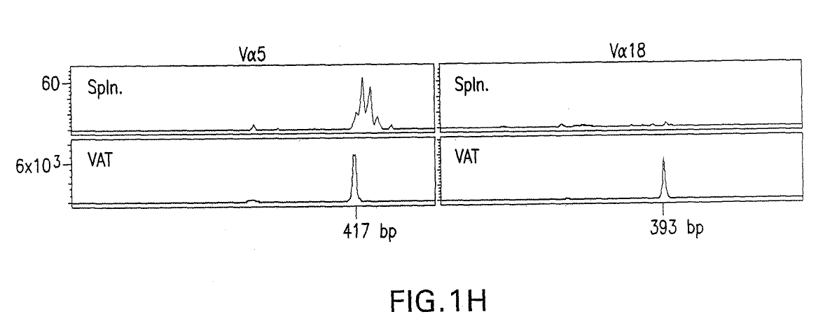

Numbers and subsets of T^{fat} from inguinal SAT and epididymal VAT from 14-18 week old diet-induced obesity (DIO) or lean (i.e., regular diet or RD) C57BL/6 (B6) mice are characterized, and 4×10⁵ CD3+ T-cells/g from visceral adipose tissue (VAT) and subcutaneous adipose tissue (SAT) are consistently harvested. The T^{fat} subset distribution varies somewhat, with a trend towards higher CD8+:CD4+ ratios, particularly in VAT of DIO mice (See FIG. 1A). VAT from lean and obese mice also contains pools of CD3+CD4/CD8 double-negative lymphocytes (i.e., NK, NKT, and Ty/δ-lineage cells). See, e.g., Caspar-Bauguil, S. et al., “Adipose tissues as an ancestral immune organ: site-specific change in obesity,” FEBS Lett 579:3487-3492 (2005), the entire contents and disclosure of which are hereby incorporated by reference. These CD3+CD4/CD8 double-negative lymphocytes are largely unaffected by obesity.

CD4+ effector T-cells may be sub-divided into (1) pro-inflammatory Th1 and Th17 sublineages, and (2) regulatory (or anti-inflammatory) Th2 and Foxp3+ T-cell sublineages. A greater number of IFNγ-secreting Th1 cells are observed in VAT than in SAT from both lean and DIO B6 mice (see FIG. 1B). Unlike mice on a regular diet, DIO mice show a strong bias among T^{fat} cells toward increasing numbers of IFNγ-secreting Th1 cells in VAT relative to SAT. Much smaller proportions of Th17 sublineage cells are observed, and these proportions of Th17 cells become even further reduced in VAT of DIO mice compared to mice on a regular diet (see FIG. 1B). The proportion of anti-inflammatory CD4+Foxp3+ cells in T^{fat} is 70% less in VAT of DIO mice relative to mice on a regular diet, thus reinforcing the pro-inflammatory VAT profiles of obese mice (see FIG. 1C). With the strong bias of Th1 cells in VAT, the ratio of Th1:Foxp3+ T cells increases from about 1.5:1 under a regular diet to about 6.5:1 in DIO mice. In terms of absolute numbers, approximately 3 times more Th1 cells accumulate in fat of DIO mice (see FIG. 1D). However, the obesity-associated increase in Th1:Foxp3+ ratio in VAT reflects mainly the accumulation of Th1 cells, rather than a reduction of Foxp3+ cells, because the absolute numbers of CD4+Foxp3+ T cells/g of VAT is maintained at approximately the same level in both regular diet and DIO mice (See FIG. 1D, lower right panel). These data suggest that the number of Th1 cells represent the dynamic variable in adipose tissue of DIO mice. Only minimal amounts of Th2 cells and associated cytokines are observed in adipose tissue of lean or DIO mice (see below).

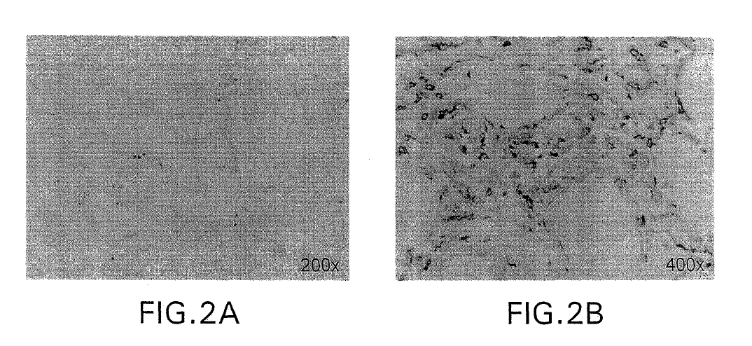

Observations in lean and obese humans (BMI>30) are analogous. In VAT from obese humans (See FIG. 1E, top right panel), Th1 cells that express the lineage-specific T-het transcription factor (stained blue) (see, e.g., Szabo, S. J. et al., “A novel transcription factor, T-bet, directs Th1 lineage commitment,” Cell 100:655-669 (2000), the entire contents and disclosure of which are hereby incorporated by reference) outnumber Foxp3+ T-cells (stained brown) by about 12:1, while the ratio is about 6:1 in normal weight humans (FIG. 1E, bottom right panel; see left panel insert of FIG. 1E for ratios). The relative proportion of Th1 to Foxp3+ cells in VAT is related to the donor body mass index (BMI) (See FIG. 1E, left panel, r=0.79). As in mice, human T^{fat} cells are observed clustered around blood vessels with some scattered between adipocytes (FIG. 2A). Foxp3+ T^{fat} are mostly observed in close proximity to monocytes or macrophages (FIG. 2B), predicting a possible interaction with fat-associated monocytes and/or macrophages.

Tissue-selective lymphocyte accumulation may reflect a number of different mechanisms, including cognate events involving T cell receptor engagements with a specific ligand. Selective TCR engagement of VAT associated T cells is not expected. However, to investigate this possibility, ovalbumin (OVA-) specific “OT2” TCR transgenic B6 mice are used. See, e.g., Barnden, M. J. et al., “Defective TCR expression in transgenic mice constructed using cDNA-based alpha- and beta-chain genes under the control of heterologous regulatory elements,” Immunol Cell Biol 76:34-40 (1998), the entire contents and disclosure of which are hereby incorporated by reference. In these mice, a small proportion of transgenic T cells by-pass TCRα allelic exclusion and undergo random, secondary rearrangements, yielding a second surface TCR that can recognize non-OVA antigens. See, e.g., Padovan, E. et al., “Expression of two T cell receptor alpha chains: dual receptor T cells,” Science 262:422-424 (1993), the entire contents and disclosure of which are hereby incorporated by reference. This population can be identified by a reduced level of original transgenic TCRα displayed on the surface of the T-cell relative to CD3 surface marker.

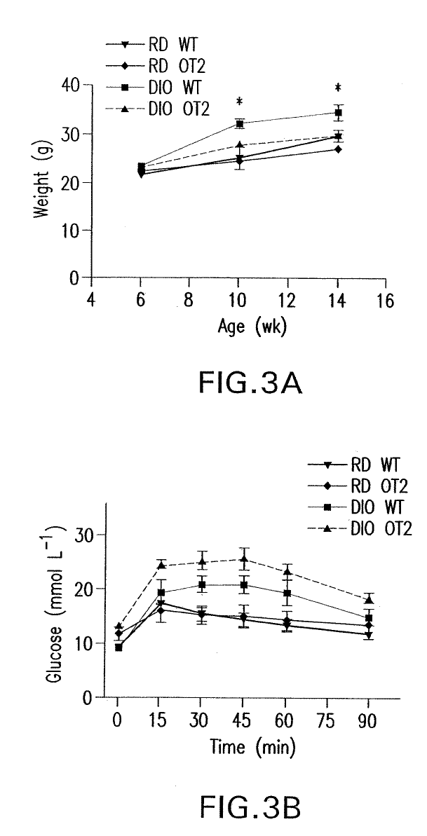

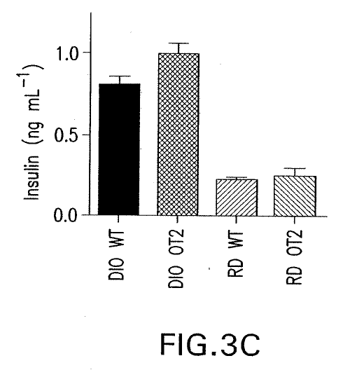

DIO-OT2 mice develop obesity, glucose intolerance and hyperinsulinemia relative to wild-type or OT2 mice on a regular diet (See FIG. 3A-C). Small pools (i.e., about 3-6%) of T cells with secondary TCRα rearrangements are found in the spleen and SAT of DIO-OT2 mice. In contrast, secondary TCRα rearrangements in VAT of DIO-OT2 mice are significantly increased (i.e., about 8-21%) in a tissue-selective manner beginning around 6 week of age and expanding over time (See FIG. 1F, p<0.001). Secondary TCRα rearrangements in VAT of DIO-OT2 mice are also significantly increased, albeit to a lesser extent, in mice on a regular diet. This tissue-specificity of TCRα rearrangements indicates a strong selective pressure towards a local non-OVA antigen(s), i.e., an adipose tissue antigen or antigen modification present only in VAT, but not in SAT or other tissues.

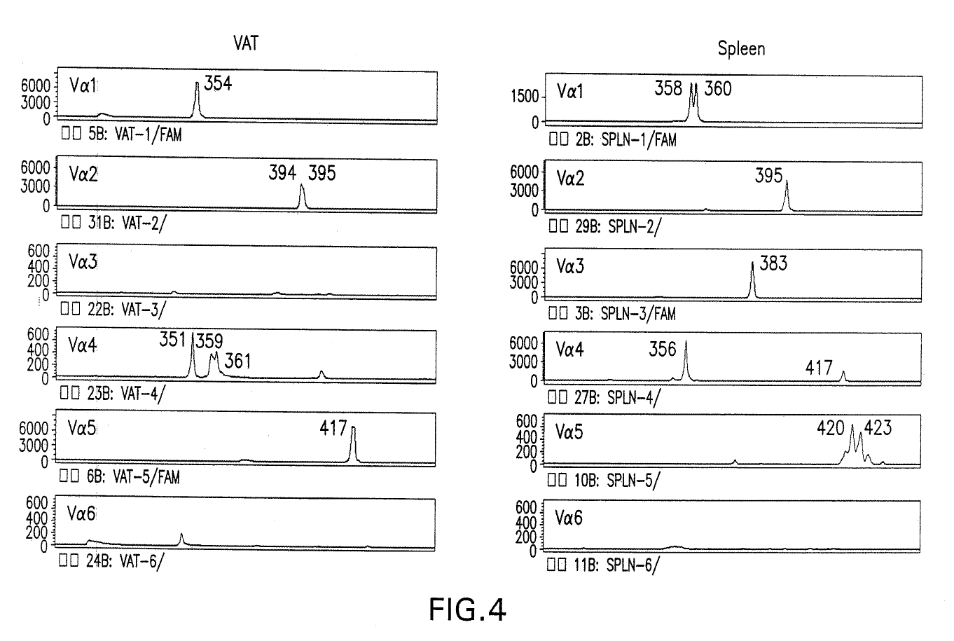

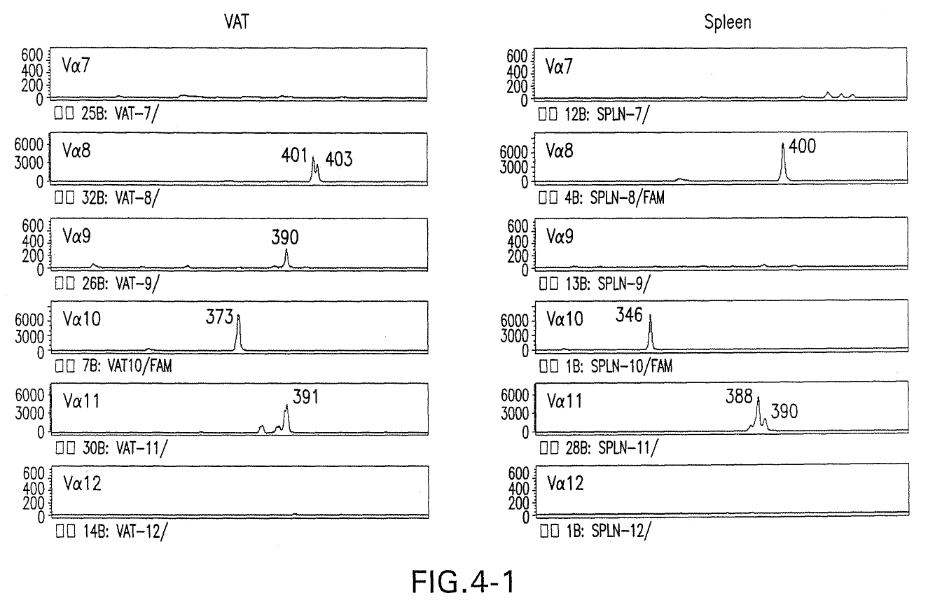

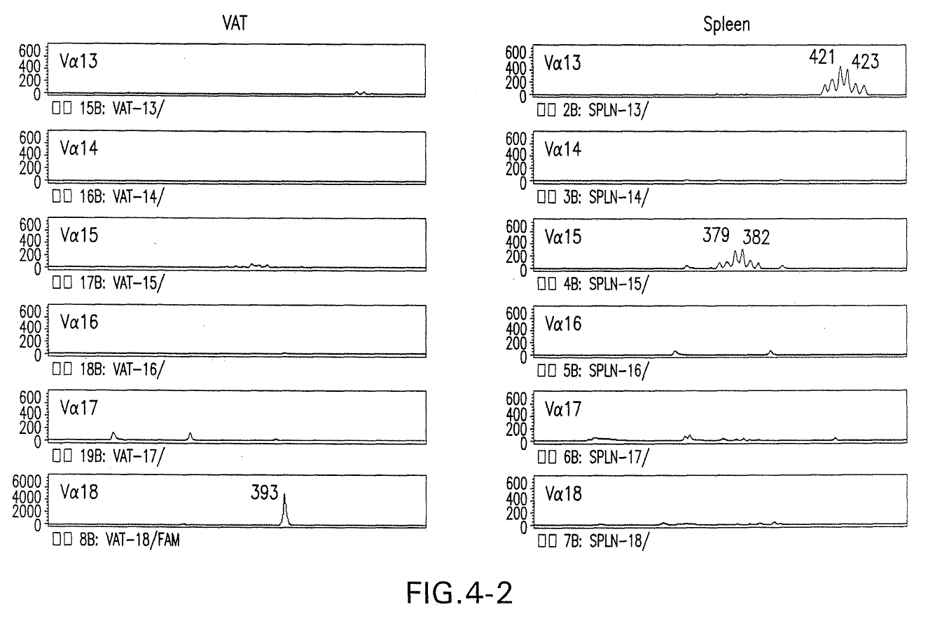

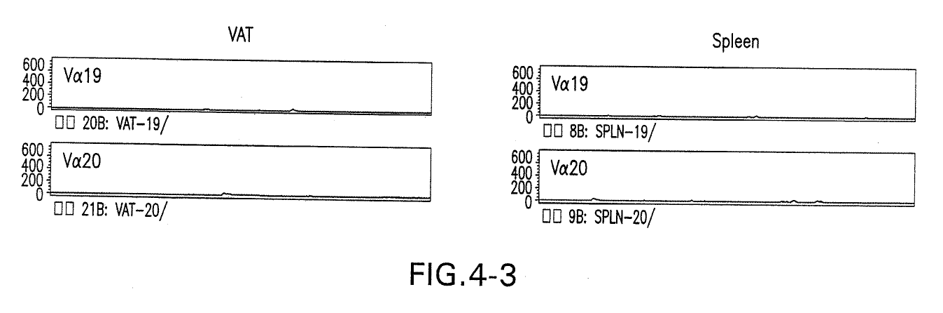

To characterize the clonal diversity of VAT- and spleen-associated T cells, each of 20 different TCRVα families in T cells expressing a secondary TCRα rearrangement are PCR amplified. These VAT-associated T-cells are sorted by flow cytometry from spleens or VAT of obese 16 week old DIO-OT2 mice (see FIG. 1G). From spleen or VAT T-cells pooled from 6-12 animals, VAT-specific TCRα re-rearrangements are observed with unique Vα5 and Vα18 families of clones that are not found in spleen. TCRα spectral typing analysis confirms the presence of VAT-associated T-cell clones containing the Vα5 (predominant clone, 417 base pairs) and Vα18 (predominant clone, 393 bp) gene segments that are essentially absent in spleen T-cells (FIG. 1H and FIG. 4) as well as less prominent VAT-associated clones in the Vα1, Vα8, Vα10 and Vα11 regions. These results indicate that the accumulation of specific VAT-associated T cell clones is due to a cognate antigen present in VAT. Tissue-driven TCR usage lies at the core of organ-selective autoimmune disorders (see, e.g., Amrani, A. et al., “Progression of autoimmune diabetes driven by avidity maturation of a T-cell population,” Nature 406:739-742 (2000), the entire contents and disclosure of which are hereby incorporated by reference) but has not previously been associated with obesity, MetSyn, or T2D.

### Example 2

### The Impact of Lymphocyte Deficiency on Obesity and Glucose Homeostasis

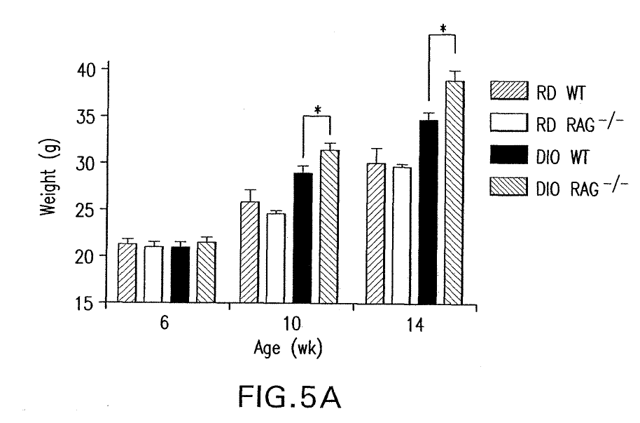

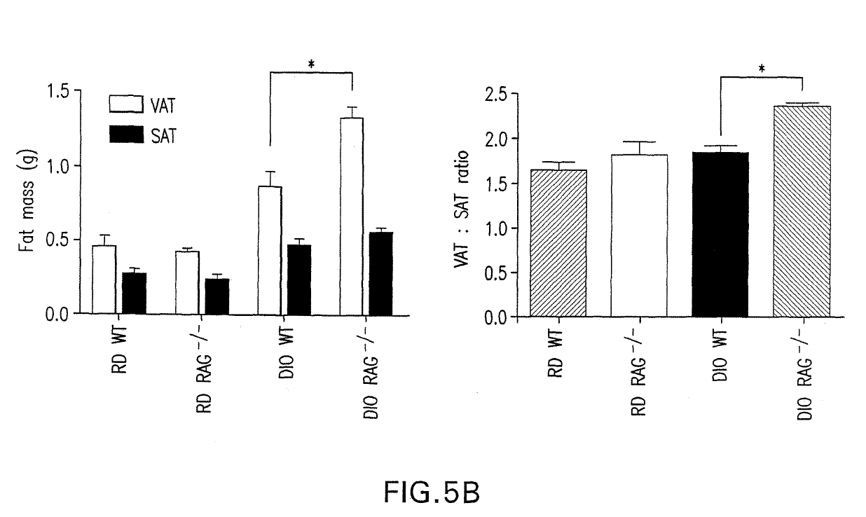

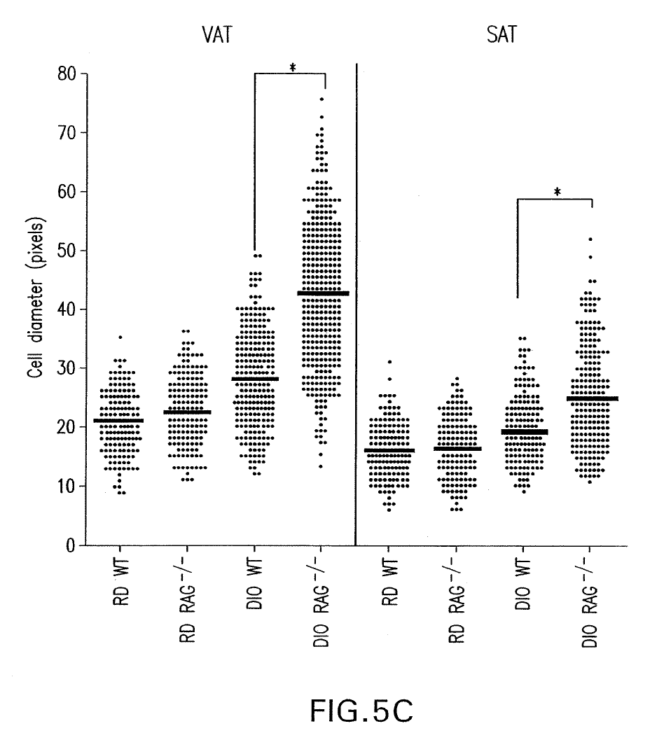

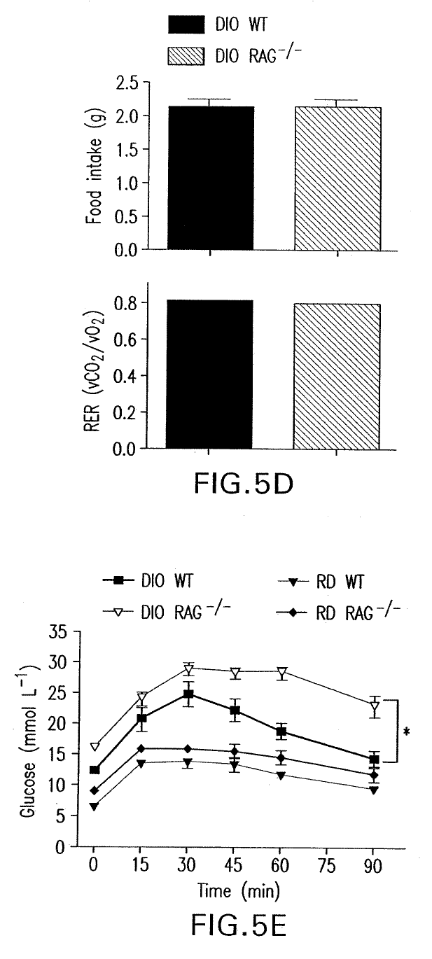

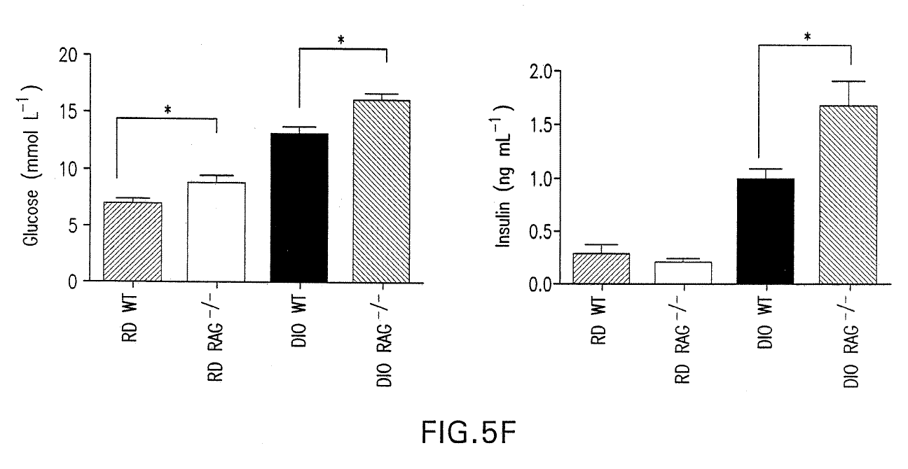

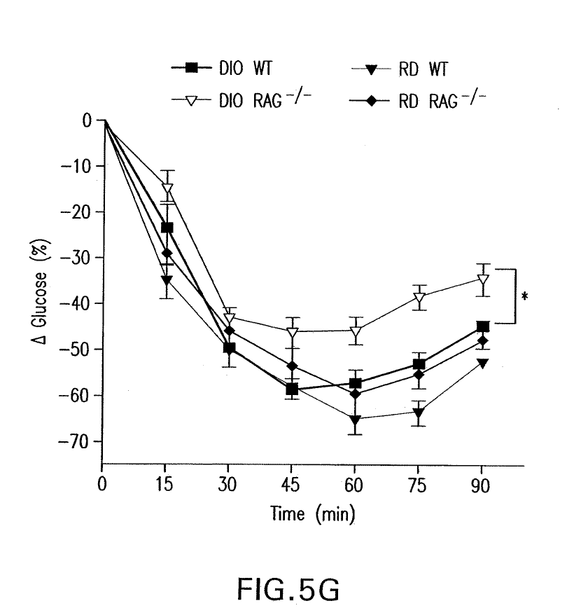

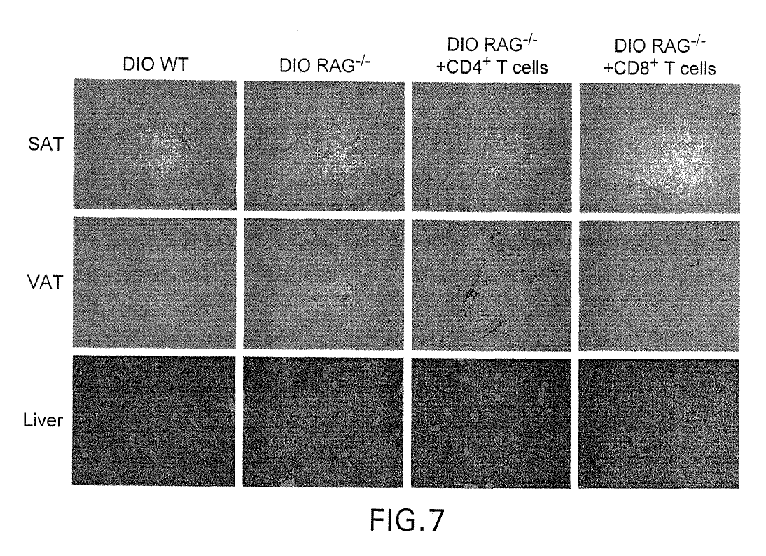

DIO-RAG^{null} mice, which lack lymphocytes, show progressively worse weight gain and visceral adiposity compared to DIO immune-competent mice (see FIGS. 5A and 5B). Increased visceral obesity in DIO-RAG^{null} mice is mostly due to impressive adipocyte hypertrophy associated with marked hepatic-steatosis (see FIG. 5C and FIG. 7). The DIO-associated RAG^{null} changes are dependent on a high fat diet because there are not any significant differences in body weight, adipose tissue mass, or adipose cell size in RAG^{null} mice fed normal diets (FIGS. 5A-C). There is also no behavioral differences in food intake or CO₂ output/VO₂ consumption between DIO-RAG^{null} and DIO-WT mice (FIG. 5D). These findings suggest that metabolic changes occurring as a consequence of high fat intake are controlled by lymphocytes.

Glucose tolerance in obese 14 week old DIO-RAG^{null} mice is severely impaired, with all glucose levels, including fasting levels of glucose, at diabetic levels (see FIG. 5E, p<0.01). Hyperinsulinemia under fasting conditions (FIG. 5F) and poor insulin sensitivity upon insulin challenge (see FIG. 5G) are also observed in obese 14 week old DIO-RAG^{null} mice. However, RAG^{null} mice do show some elevation in fasting blood glucose levels (FIGS. 5E-G, p<0.01). The triad of glucose intolerance, hyperinsulinemia, and poor insulin sensitivity are metabolic derangements related to MetSyn which may result from chronically elevated levels of insulin. Indeed, these DIO-RAG^{null} mice also exhibit a significant increase in phosphorylated JNK and a decrease in phosphorylated STAT3 in adipose tissue (FIG. 5H), a constellation closely linked to insulin resistance. See, e.g., Hirosumi, J. et al., “A central role for INK in obesity and insulin resistance,” Nature 420:333-6 (2002); and Inoue, H. et al., “Role of STAT-3 in regulation of hepatic gluconeogenic genes and carbohydrate metabolism in vivo,” Nat Med 10:168-74 (2004), the entire contents and disclosures of which are hereby incorporated by reference. In addition, DIO-RAG^{null} mice show uncontrolled macrophage activation, as evident from elevated serum levels of MCP-1, TNFα, and IL6 molecules that have been associated with insulin-resistance (FIG. 5I). See Weisberg, S. P. et al., supra (2006); Febbraio, M. A. supra (2007); and Hotamisligil, G. S. et al., “Adipose expression of tumor necrosis factor-alpha: direct role in obesity-linked insulin resistance,” Science 259:87-91 (1993), the entire contents and disclosures of which are hereby incorporated by reference. In contrast, RAG^{null} mice on a regular diet only show small and insignificant trends towards poorer glucose tolerance and increased insulin resistance. These observations suggest that the metabolic role of T-cell lineages is physiological and that T-cells function even under normal caloric diets (i.e., without the challenge of a hypercaloric diet).

### Example 3

### CD4+ T Cells Regulate Obesity and Glucose Homeostasis

The DIO-RAG^{null} phenotype suggests a protective role of lymphocytes in obesity and associated insulin resistance. To identify lymphocyte subtypes involved in this process, 12 week old DIO-RAG^{null} mice are reconstituted with about 5×10⁶ CD4+ or CD8+ T-cells, each specifically depleted of CD49b+NK/NKT-cells, CD11b+ monocytes or macrophages, and CD19+ and CD45R+ B-cells prior to transfer.

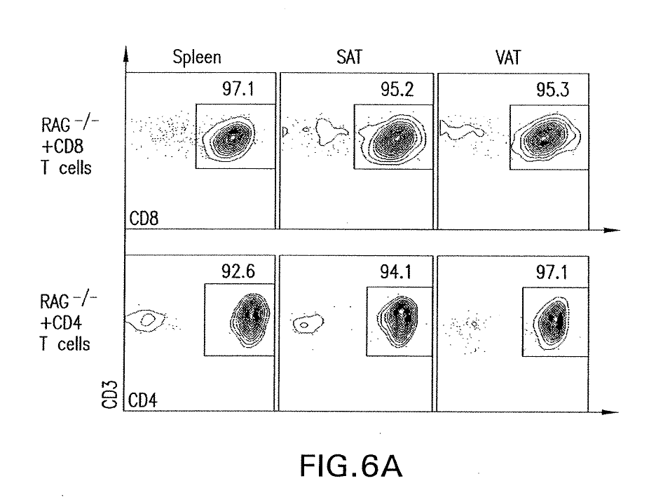

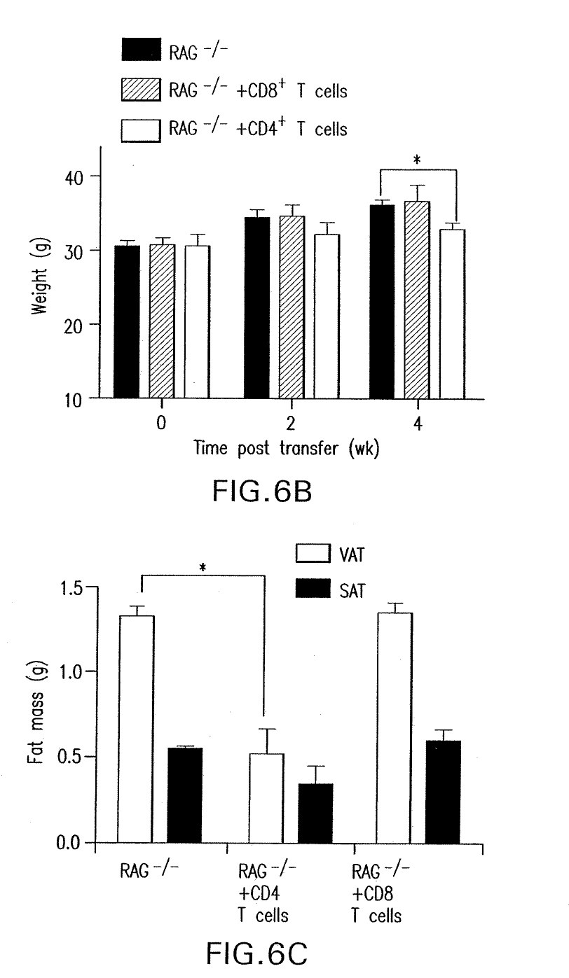

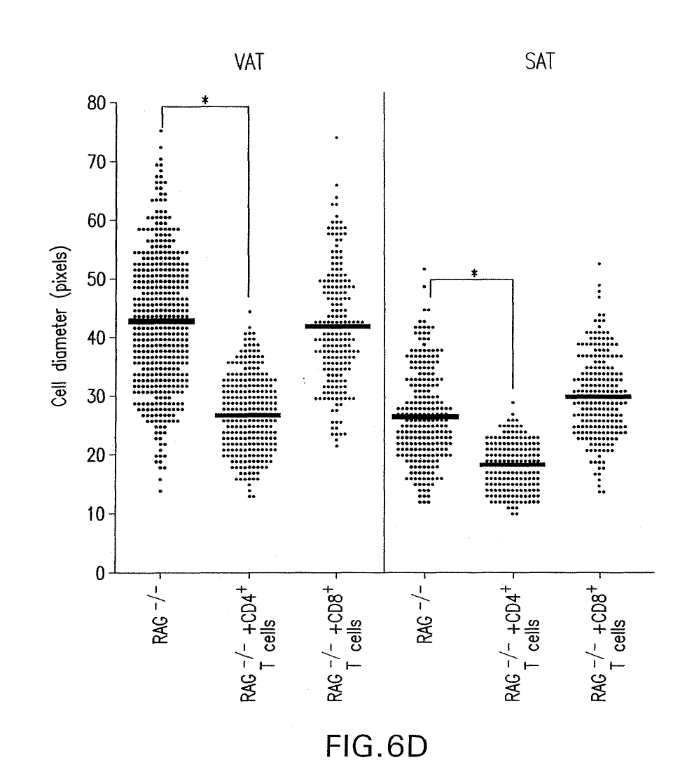

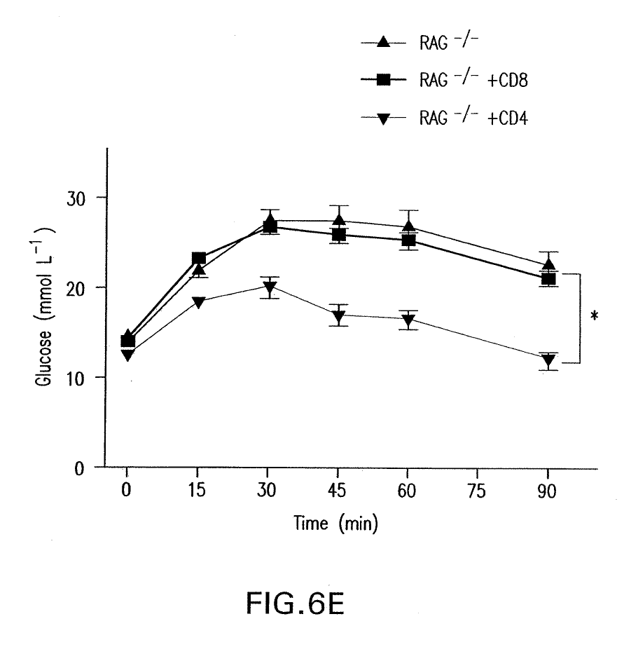

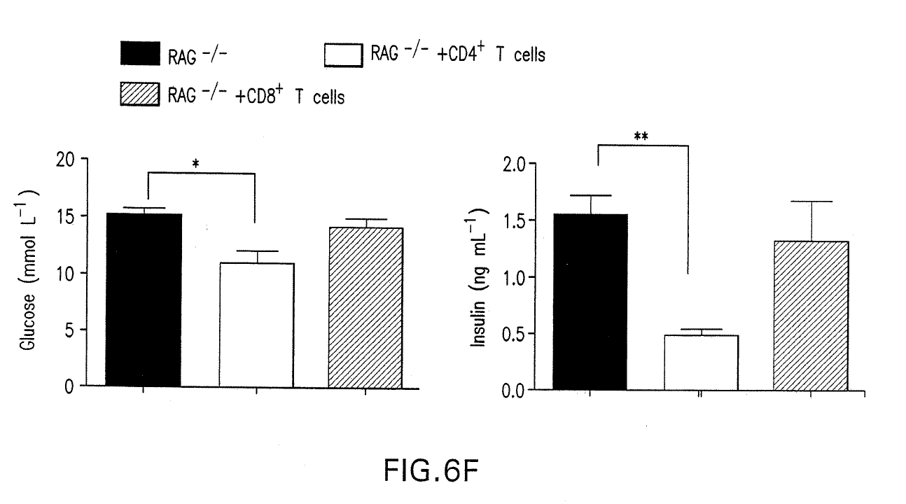

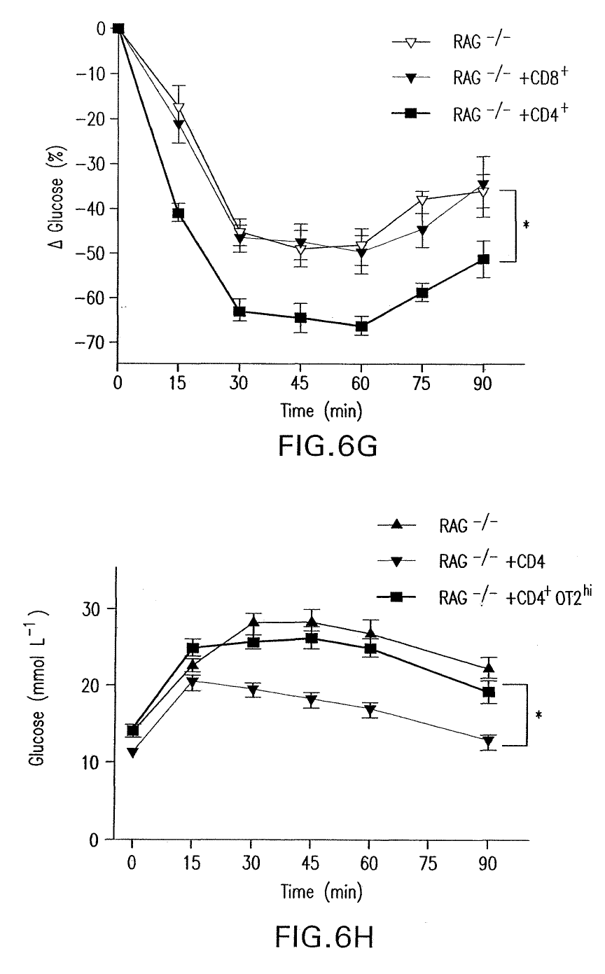

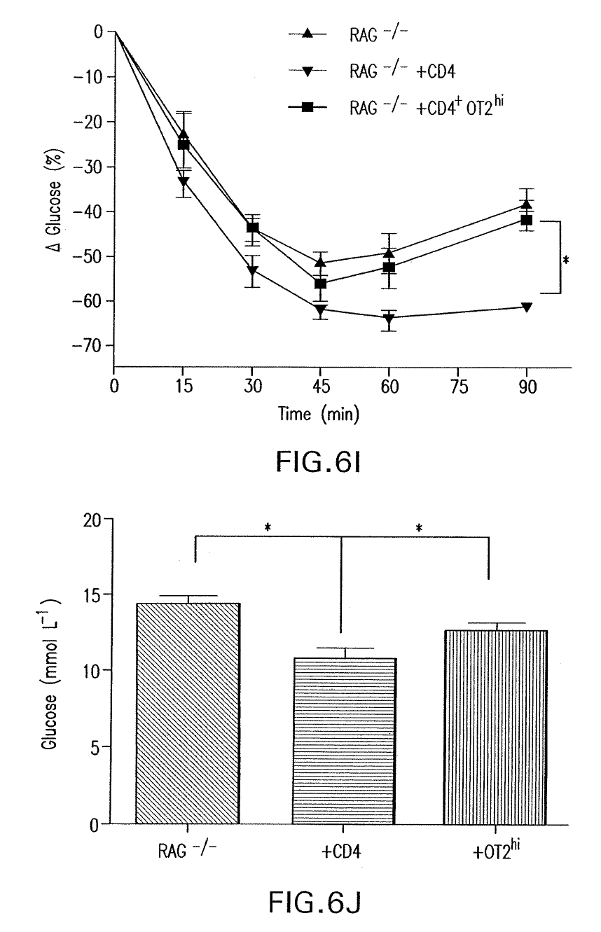

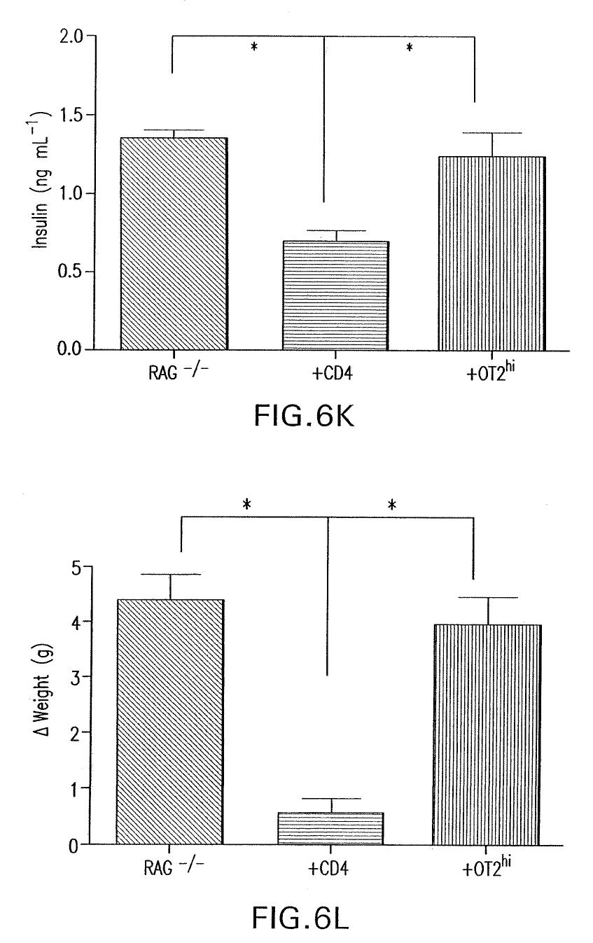

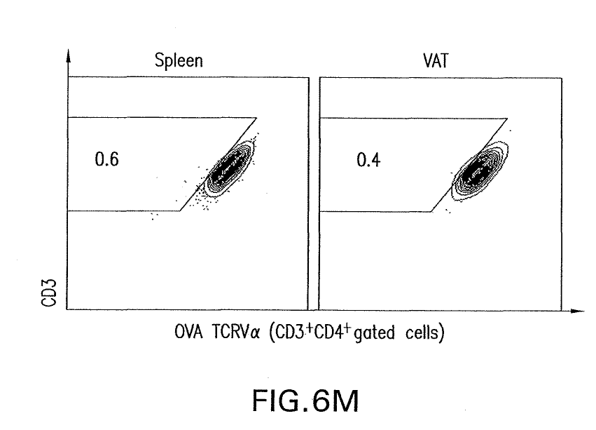

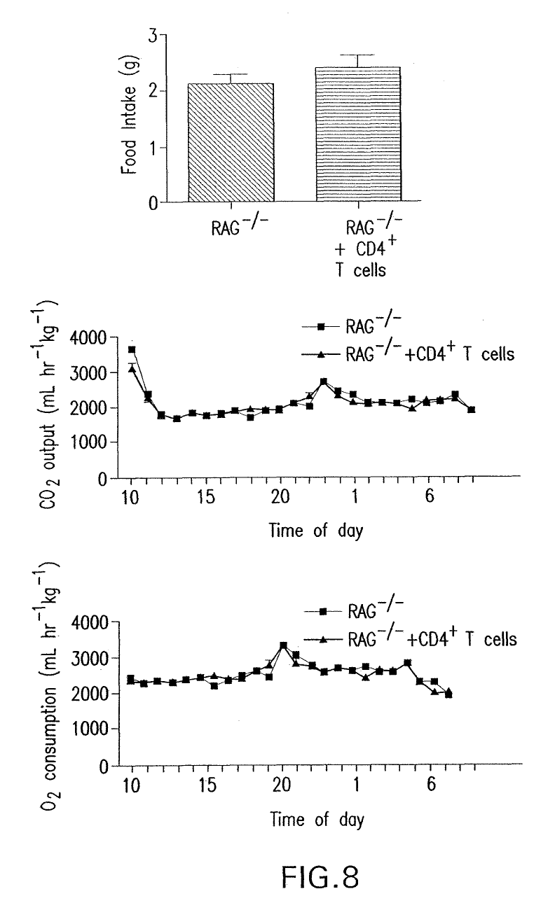

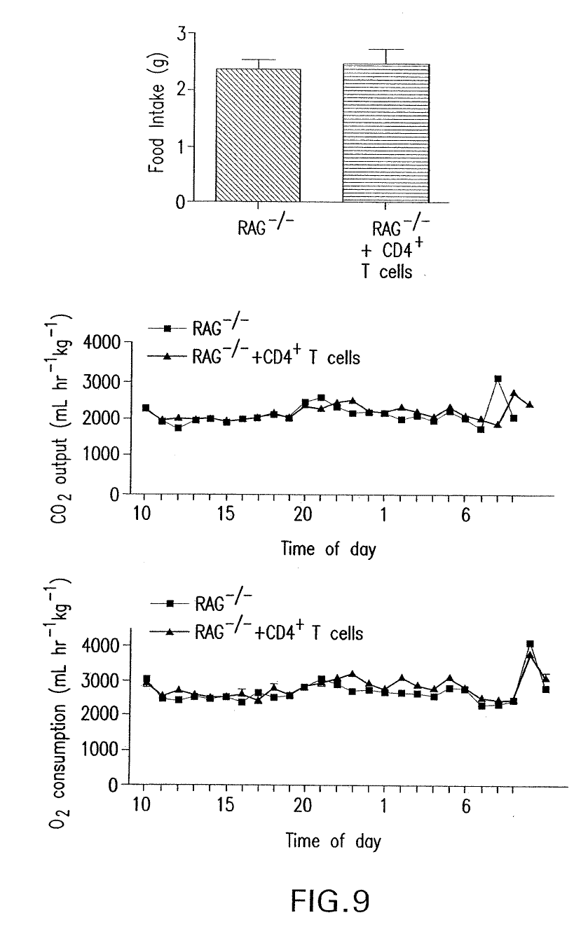

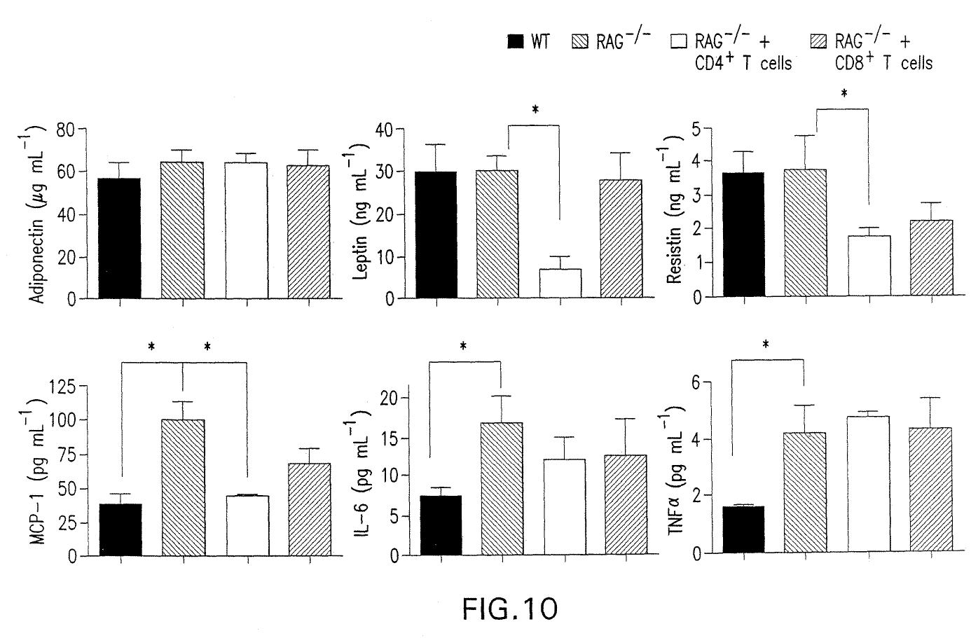

Two weeks following transfer, cells have populated spleen, SAT, and VAT with good purity (i.e., about 95%, see FIG. 6A) despite the massive homeostatic expansion required in the new host. In contrast to DIO-RAG^{null} mice re-constituted with CD8+ T cells, DIO-RAG^{null} mice re-constituted with CD4+ T cells failed to gain weight above control levels, even after 2-4 weeks post transfer (see FIG. 6B, p=0.02), which may be due to a loss of VAT and, to a lesser extent, SAT (see FIG. 6C) reflecting smaller adipocyte size in VAT and SAT (see FIG. 6D and FIG. 7) as well as a reduction in hepatosteatosis (see FIG. 7) in CD4+ T-cell reconstituted RAG^{null} mice compared to CD8+ reconstituted or RAG^{null} control mice. This protective effect on the rate of weight gain is not caused by reduced food intake or any obvious metabolic shifts in CO₂ output or O₂ consumption (FIGS. 8 and 9). Unlike CD8+ T-cells, transfer of CD4+ T cells also significantly reduces serum levels of obesity-associated adipokines, leptin, resistin, and MCP-1 (FIG. 10) Importantly, grafting of CD4+ cells into DIO-RAG^{null} mice reverses impaired glucose tolerance, lower fasting levels of blood insulin and glucose, and improve insulin sensitivity within about 2 weeks compared to DIO-RAG^{null} control mice (see FIGS. 6E-G, p<0.01). Therefore, CD4+ T cells are able to protect against weight gain, insulin resistance, adipocyte hypertrophy, fatty liver damage, and changes in the levels of associated adipokines.

The beneficial effects of CD4+ T cell transfer are dependent on TCR specificity. Using a B6 “OT2” TCR transgenic mouse model, OVA-specific T cells are highly purified by sorting cells without secondary rearrangements but with high surface expression levels of the OT2 OVA-specific TCR. Two weeks following adoptive transfer of these CD4+ OT2:TCRα^{hi} lymphocytes into RAG^{null} mice, very few, if any, T-cells with secondary rearrangements in spleens or fat tissue are observed (FIG. 6M). Within the observation period, these OVA-specific T-cells failed to improve glucose tolerance (see FIG. 6H), insulin sensitivity (see FIG. 6I) fasting blood glucose (see FIG. 6J), fasting insulin levels (see FIG. 6K), and body weight (see FIG. 6L, all p values<0.02). These observations further suggest that secondary rearrangements occurring locally in adipose tissue are necessary for the beneficial immune-regulatory effects on obesity and insulin sensitivity.

### Example 4

### Regulatory T-Cells Regulate Inflammation and Autoimmunity

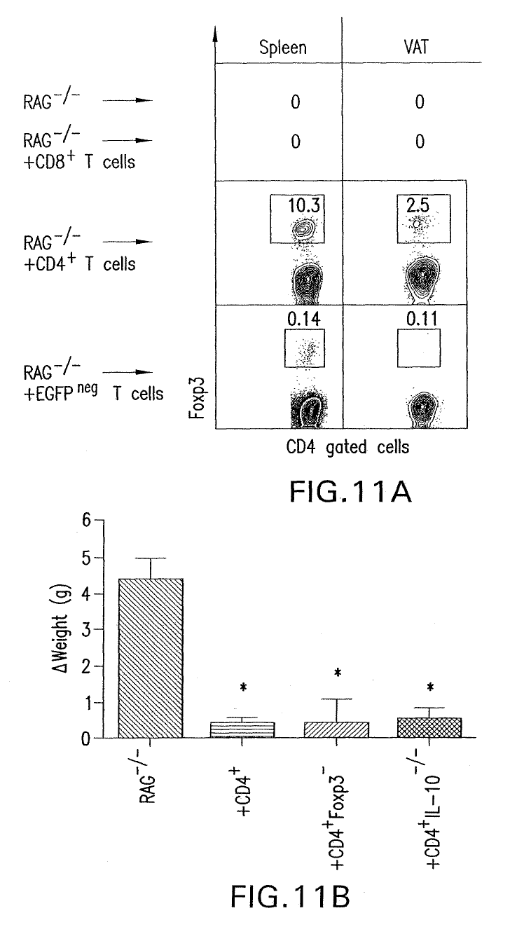

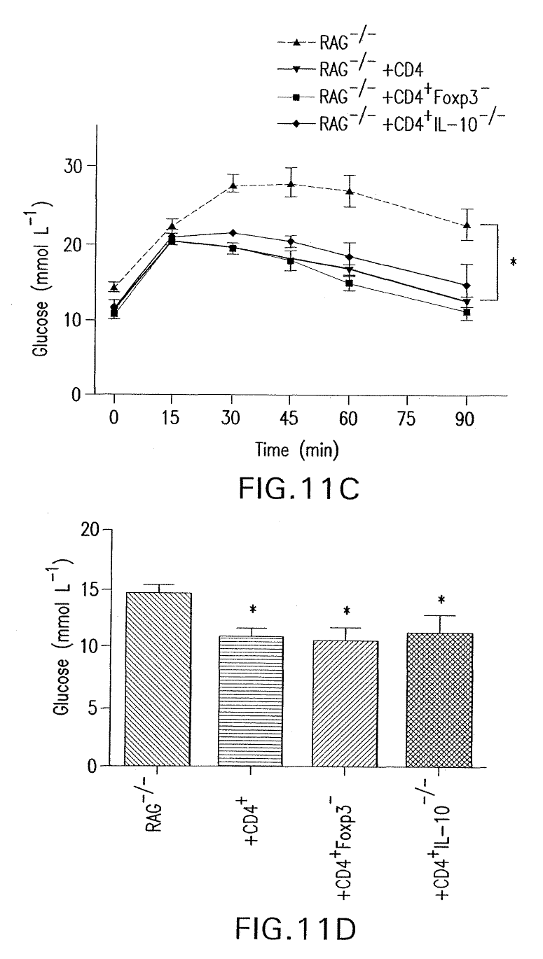

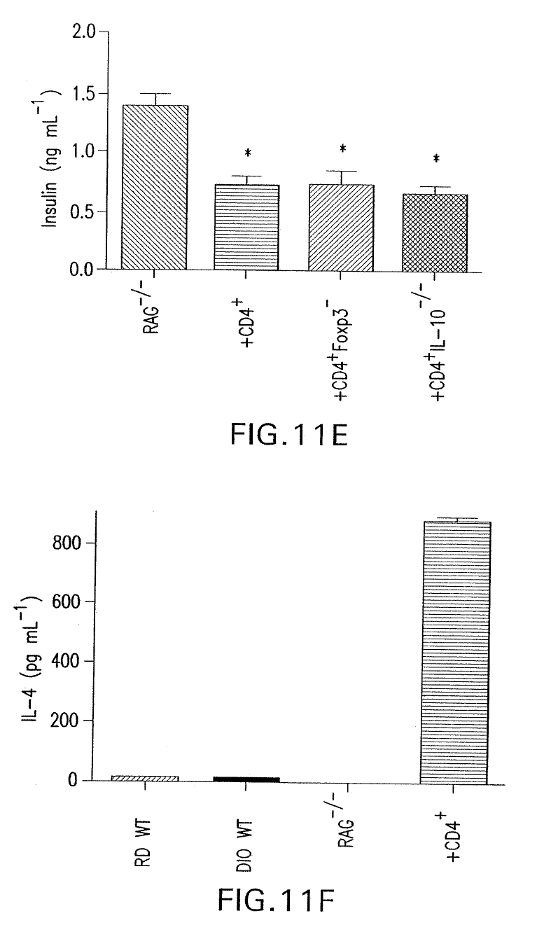

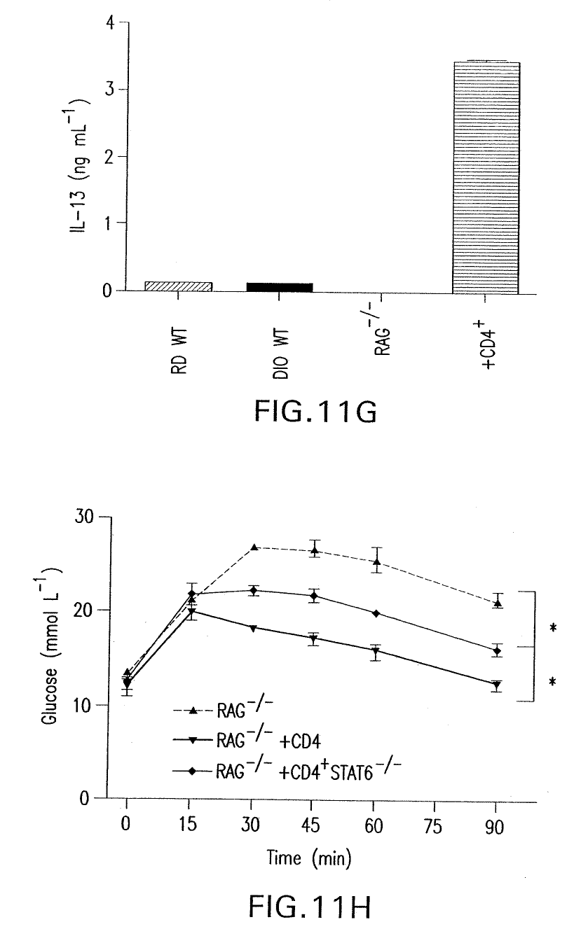

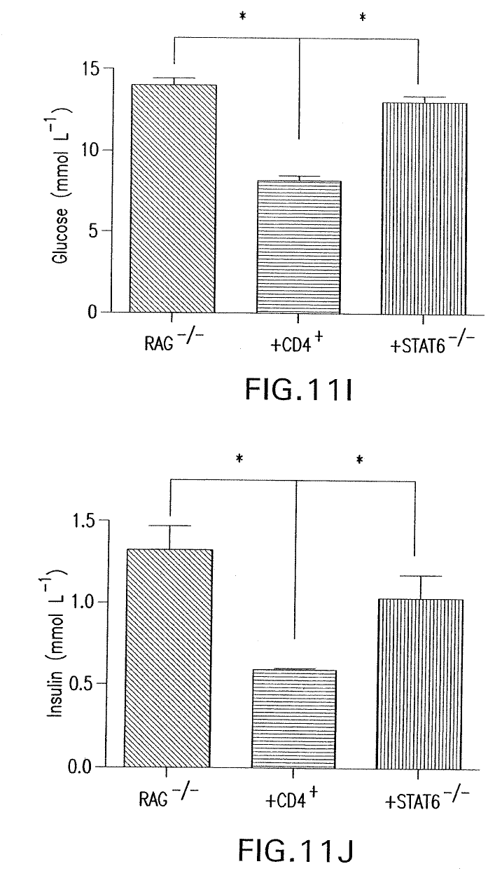

Transfer of CD4+ T cells into RAG^{null} mice slowly re-populated VAT of RAG^{null} mice with CD4+Foxp3+ T cells (i.e., about 2-3% by 2 weeks post transfer) (see FIG. 11A). CD4+Foxp3− T cells are highly purified by cell sorting, using the bi-cistronic Foxp3− EGFP transgenic B6 mouse line, where Foxp3+ cells co-express EGFP. See, e.g., Fontenot, J. D. et al., “A function for interleukin 2 in Foxp3-expressing regulatory T cells,” Nat Immunol 6:1142-1151 (2005), the entire contents and disclosure of which are hereby incorporated by reference. Transfer of these Foxp3− CD4+ T cells into DIO-RAG^{null} hosts, showed little (i.e., about 0.1%) conversion to CD4+Foxp3+ cells in spleen or VAT (see FIG. 11A, bottom). Mice reconstituted with Foxp3− CD4+ T cells are protected from rapid weight gain to a similar extent as mice receiving graft of the full CD4+ T-cell compartment from WT hosts (see FIG. 11B). Transfer of CD4+Foxp3− T-cells improve glucose tolerance as well as fasting glucose and insulin levels (FIGS. 11C-E), suggesting that metabolic control following CD4+ T cell transfer may not fully require CD4 Foxp3+ regulatory T cell function. In addition to AAM, regulatory T cells and inducible type 1 regulatory T-cells (Tr1) can express IL-10, a major anti-inflammatory cytokine, as part of their effector function. See, e.g., Awasthi, A. et al., “A dominant function for interleukin 27 in generating interleukin 10-producing anti-inflammatory T cells,” Nat Immunol 8:1380-1389 (2007), the entire contents and disclosure of which are hereby incorporated by reference. Adoptive transfer of CD4+ T cells from IL10^{null} B6 mice is as protective as transfer of the full WT CD4+ grafts, indicating that this cytokine is dispensable for the beneficial effects of CD4+ T cell transfer in this model (FIGS. 11B-E). However, it is important to note that the IL-10 gene is missing only from the transferred CD4+ T cells, but remains intact in the recipient RAG^{null} recipient mice. Therefore, other cells of the immune system, such as monocytes and/or macrophages (including AAMs), of the recipient mice are still be able to express IL-10.

T cells may acquire a Th2 profile during post-transfer homeostatic expansion of T-cells. Indeed, considerable Th2 cytokine production (IL-4 and IL-13) in VAT-derived T-cells of RAG^{null} mice reconstituted with CD4+ T cells is observed (see FIGS. 11F and 11G). Maintenance of Th2 cells is dependent on STAT6, and STAT6 deficiency severely impairs Th2 cell production. See, e.g., Shimoda, K. et al., “Lack of IL-4-induced Th2 response and IgE class switching in mice with disrupted Stat6 gene,” Nature 380:630-633 (1996); and Kaplan, M. H. et al., “Stat6 is required for mediating responses to IL-4 and for development of Th2 cells.” Immunity 4:313-319 (1996), the entire contents and disclosures of which are hereby incorporated by reference. To study the role of Th2 cells, CD4+ STAT6−/− T cells are transferred into DIO-RAG^{null} mice. Transfer of CD4+ T-cells into DIO-RAG^{null} mice shows improved glucose tolerance, improved fasting glucose and insulin levels, and reduced weight gain to a greater extent than transfer of CD4+ STAT6−/− T cells into DIO-RAG^{null} mice (FIGS. 11H-K), thus suggesting a protective role for Th2 cells in averting obesity and metabolic dysfunction. Therefore, strategies that increase Th2 cell content in VAT may have therapeutic benefit in treating MetSyn and/or T2D.

### Example 5

### Immunotherapy for Insulin Resistance

The immune-mediated regulation of adipose tissue mass, inflammation, and associated metabolic parameters relating to glucose and insulin metabolism are complex and likely involve multiple mechanisms and levels of control. Data obtained in OT2 mice demonstrate the need for cognate elements in T cell-mediated metabolic events. These observations suggest that DIO may be a fat-selective autoimmune condition.

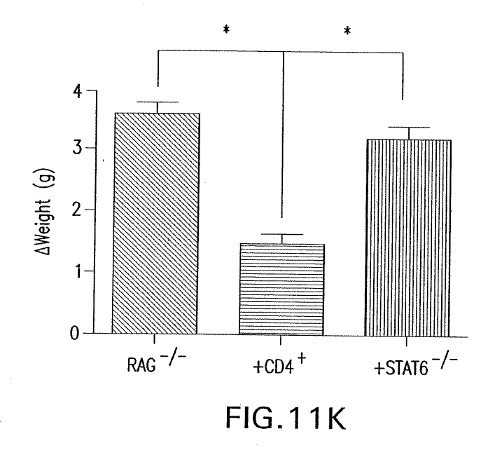

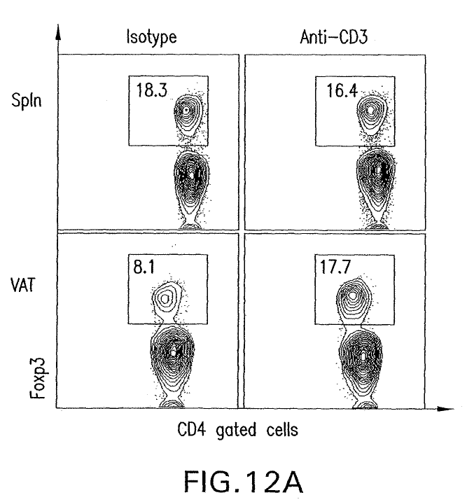

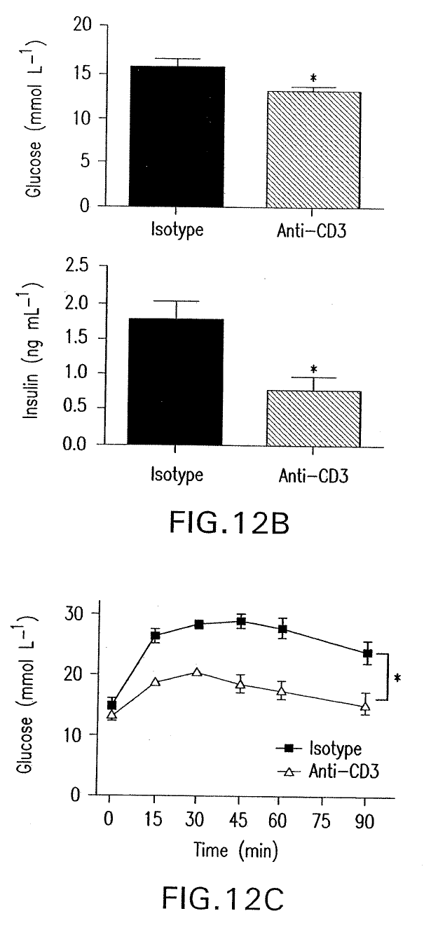

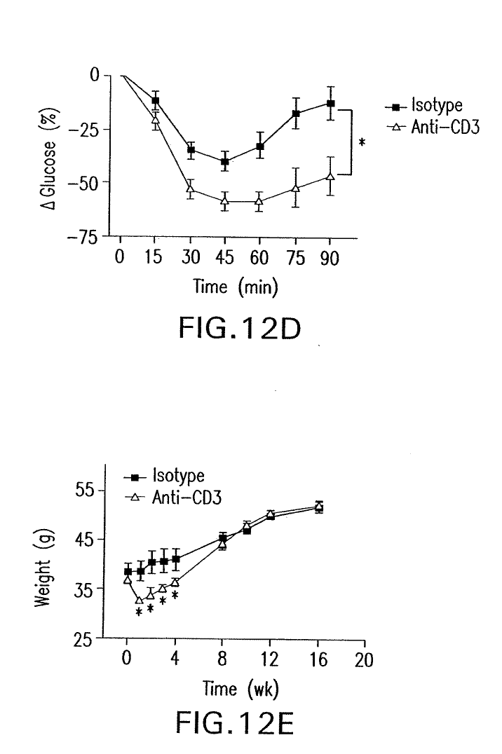

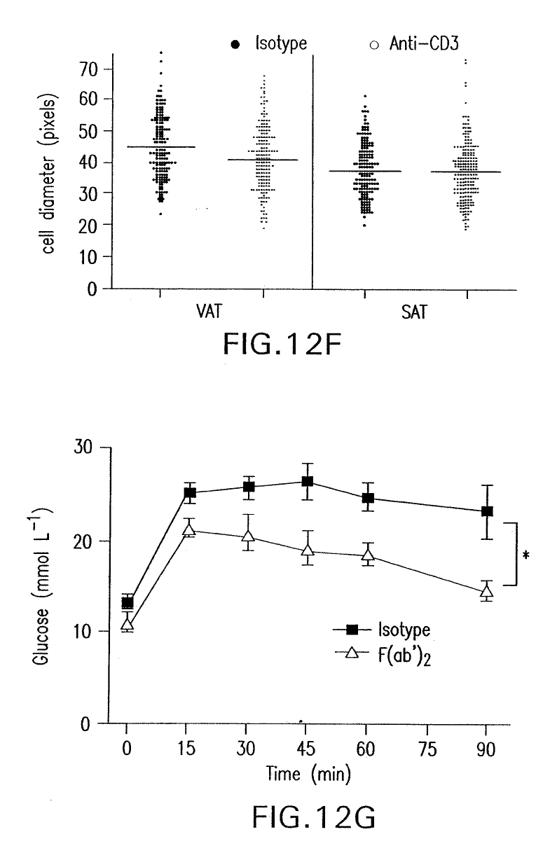

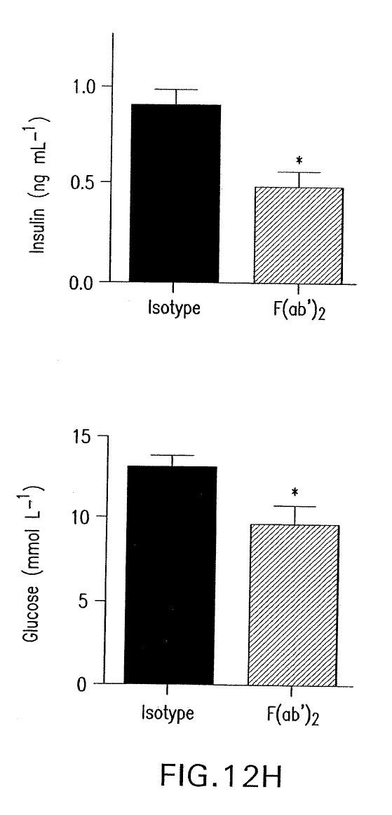

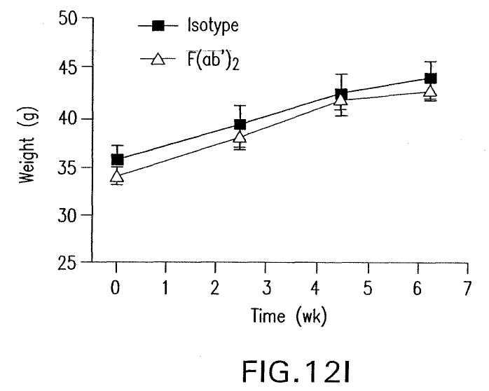

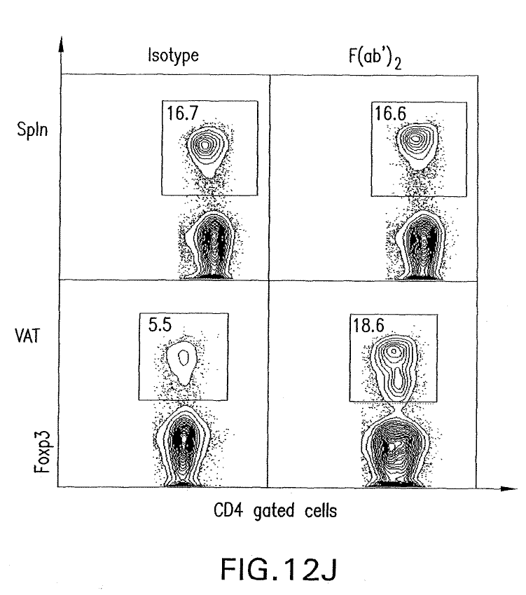

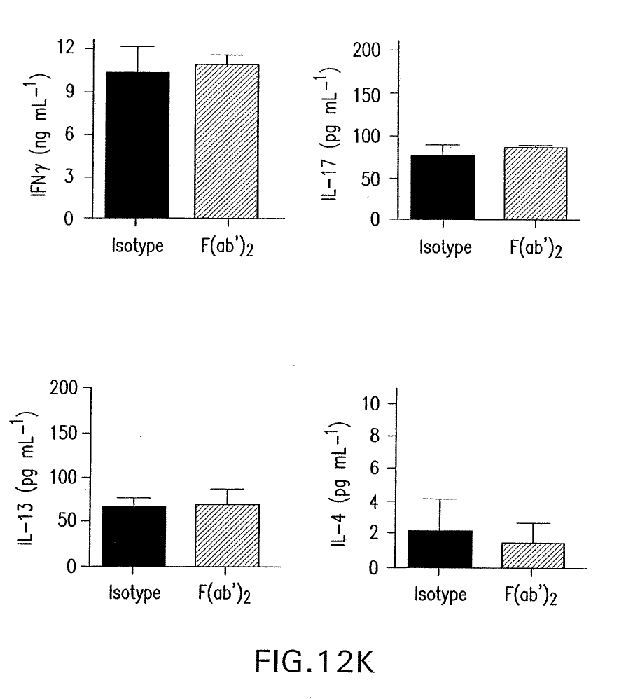

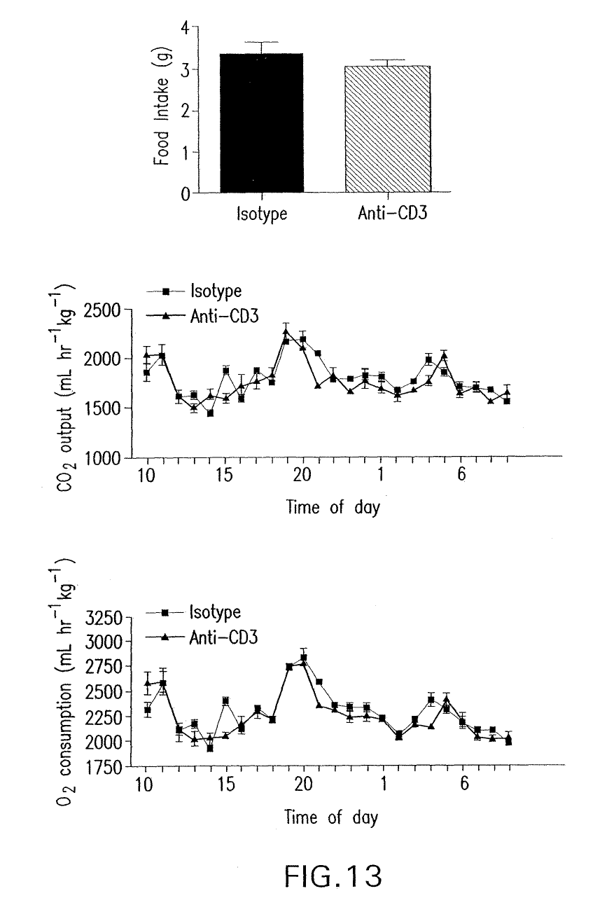

Data presented herein shows that regulatory T-cells in adipose tissue may function to reduce symptoms of glucose intolerance, hyperinsulinemia, insulin resistance, and obesity in animals. To study the effects of immune-modulatory treatments on metabolic symptoms of DIO mice, 10 μg of T cell specific αCD3 antibody is administered daily for 5 days by intra-peritoneal (i.p.) injection into 14 week old DIO-WT mice. See, e.g., Chatenoud, L. et al., supra (2007), the entire contents and disclosure of which are hereby incorporated by reference. Treatment with αCD3 antibody re-establishes T cell tolerance through a global, but transient (e.g., less than or equal to about 3 weeks), T cell depletion follows by a selective increase of CD4+Foxp3+ T cell pools at sites of tissue inflammation. See, e.g., Belghith, M. et al., “TGF-beta-dependent mechanisms mediate restoration of self-tolerance induced by antibodies to CD3 in overt autoimmune diabetes,” Nat Med 9:1202-1208 (2003); and Bisikirska, B. et al., “TCR stimulation with modified anti-CD3 mAb expands CD8+ T cell population and induces CD8+CD25+ Tregs,” J Clin Invest 115:2904-2913 (2005), the entire contents and disclosures of which are hereby incorporated by reference. Nine weeks after treatment with αCD3 antibody, DIO mice have control levels of CD4+Foxp3+ T cells in VAT (see FIG. 12A), improved fasting glucose and insulin levels, and greatly improved glucose tolerance and insulin sensitivity (see FIGS. 12B-D). There is a transient weight loss lasting about 3-4 weeks after αCD3 injection (see FIG. 12E), but this may be attributable to a post-activation “cytokine storm” mediated largely by TNFα. See, e.g., Alegre, M. et al., “Acute toxicity of anti-CD3 monoclonal antibody in mice: a model for OKT3 first dose reactions,” Transplant Proc 22:1920-1921 (1990); and Alegre, M. et al., “Hypothermia and hypoglycemia induced by anti-CD3 monoclonal antibody in mice: role of tumor necrosis factor,” Eur J Immunol 20:707-710 (1990), the entire contents and disclosures of which are hereby incorporated by reference. There is no significant difference in body weight, adipocyte hypertrophy, food intake, or CO₂/O₂ production/consumption 9 weeks following αCD3 treatment (see FIGS. 12E and 12F and FIG. 13).

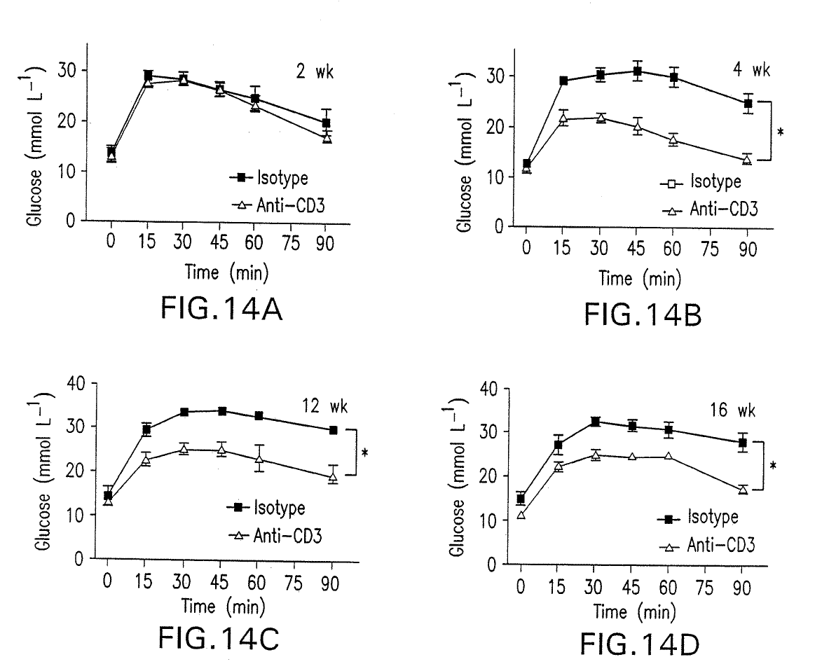

If the beneficial effects of αCD3 treatment are mediated by restored VAT levels of regulatory T cells, then improved insulin resistance and glucose tolerance may be expected. Indeed, the beneficial effects of αCD3 treatment on glucose metabolism began at about 4 weeks after injection and lasted the entire observation period (i.e., greater than or equal to about 4 months, FIGS. 14A-D). Thus, treatment with αCD3 antibody seems to provide a positive and lasting effect on metabolic T-cell functions in adipose tissue. A similar, but slightly less effective, therapeutic effect of αCD3 immunotherapy is observed in diabetic, leptin-deficient (i.e., ob/ob) mice (data not shown). This may be explained by complex interactions between leptin and regulatory T cells. See, e.g., De Rosa, V. et al., “A key role of leptin in the control of regulatory T cell proliferation,” Immunity 26:241-255 (2007), the entire contents and disclosure of which are hereby incorporated by reference.

The adverse effects of the “cytokine storm” may limit the use of intact αCD3 antibody therapy for human use. Therefore, the therapeutic effects of a non-mitogenic F(ab′)₂ fragment of αCD3 is tested. See, e.g., Chatenoud, L. et al., supra (2007), the entire contents and disclosure of which are hereby incorporated by reference. About 150 μg of F(ab′)₂ may be injected per day for 5 days, resulting in long-term improvements in glucose tolerance as well as fasting glucose and insulin levels (see FIGS. 12G and 12H) with no initial weight loss observed (see FIG. 12I). The F(ab′)₂ treatment does show restored numbers of CD4+Foxp3+ T-cells in VAT (FIG. 12J), but no change is observed in the levels of Th1, Th17, or Th2 cytokines produced by VAT T-cells after the F(ab′)₂ therapy (FIG. 12K). Thus, a brief and relatively non-toxic treatment protocol using a F(ab′)₂ fragment of αCD3 antibody may be used to restore VAT regulatory T-cell pools to improve long term glucose and insulin homeostasis in insulin resistant patients.

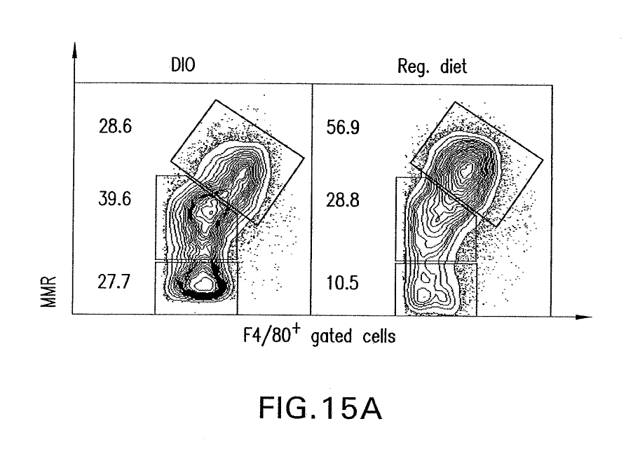

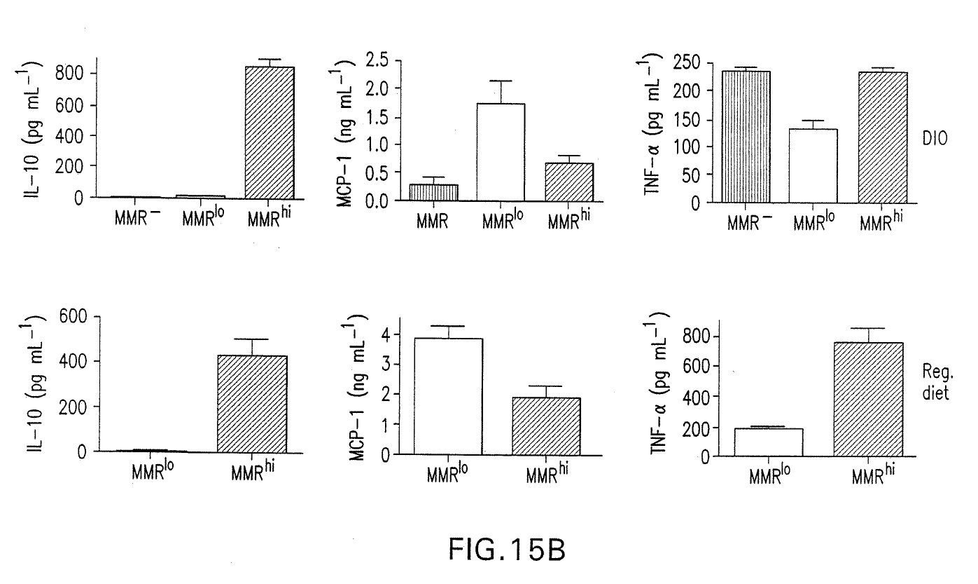

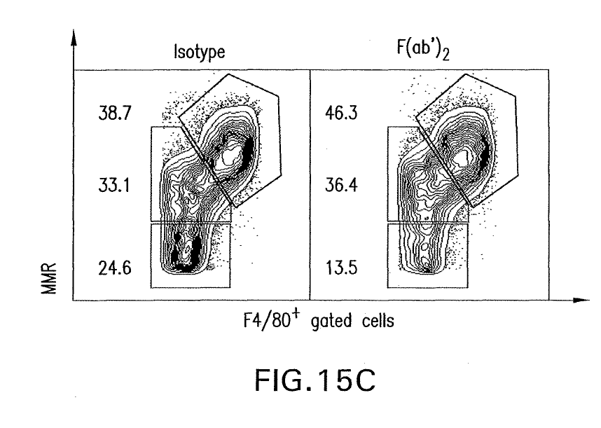

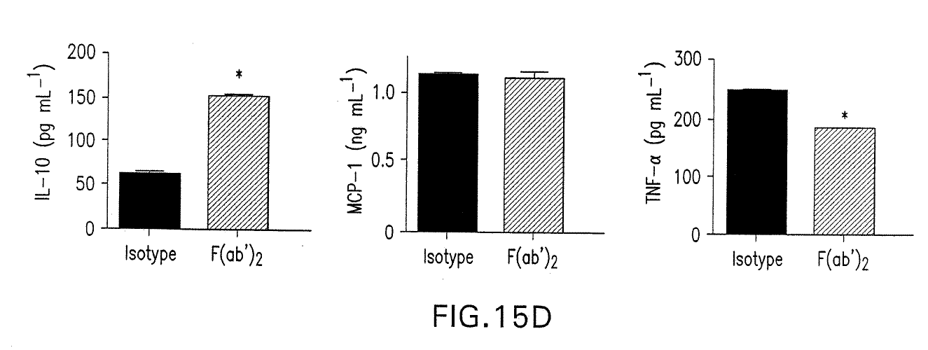

CD4+Foxp3+ T cells may induce macrophages to differentiate into M2-like “alternatively activated” macrophages (AAM) that secrete IL-10, which may protect against insulin resistance. See, e.g., Tiemessen, M. M. et al., supra (2007); and Zeyda, M. et al., “Adipose tissue macrophages.” Immunol Lett 112:61-67 (2007), the entire contents and disclosures of which are hereby incorporated by reference. To determine whether restoration of VAT regulatory T-cells by αCD3 immunotherapy generates IL-10-secreting M2 macrophages, cytokine profiles of M2 macrophages based on surface staining of MMR are examined. Three unique macrophage (F4/80+ cells) populations are identified in VAT of both DIO and regular mice: MMR^{neg}, MMR^{lo}, and MMR^{hi} (FIG. 15A). Only MMR^{hi} macrophages express IL-10, while MMR^{lo} macrophages express the most MCP-1 and MMR^{neg} macrophages (likely representing a classical M1 macrophages) express MCP-1 and TNFα (FIG. 15B).

Interestingly, diet has little affect on the pattern of expression of these cytokines (FIG. 15B). However, DIO mice do have increased proportions of TNFα-secreting MMR^{neg} macrophages and reduced proportions of IL-10-secreting MMR^{hi} macrophages (FIG. 15A). F(ab′)₂ therapy increased the MMR^{hi} pool and reduced the MMR^{neg} pool by about 6 weeks after treatment (FIG. 15C). This macrophage shift generated an approximately 300% increase in IL-10 production as measured in purified VAT macrophages from F(ab′)₂ treated mice (FIG. 15D). Therefore, F(ab′)₂ therapy may have a dual function or benefit of increasing both the level of anti-inflammatory T-cells as well as the levels of IL-10-secreting macrophages.

### Example 6

### αCD3 Treatment Causes T-Cell Secretion of Leukemia Inhibitory Factor (LIF)

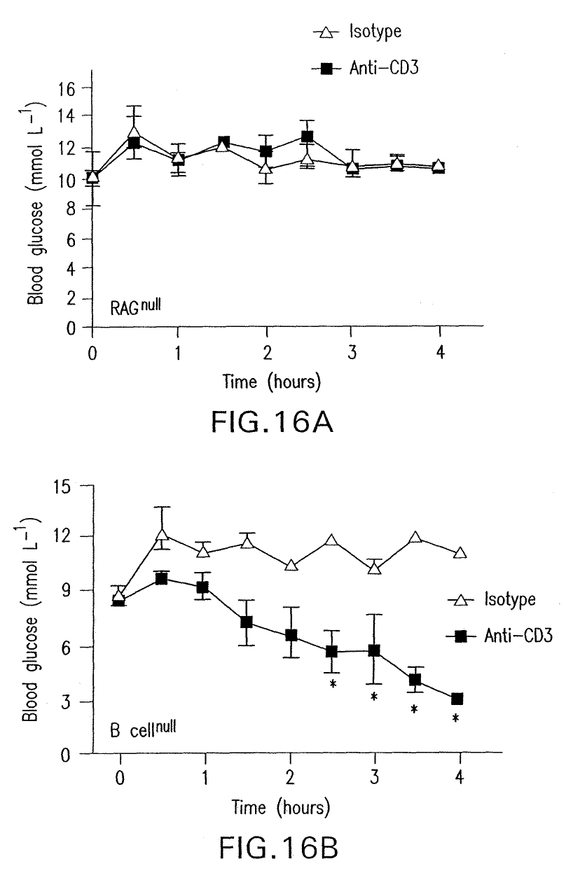

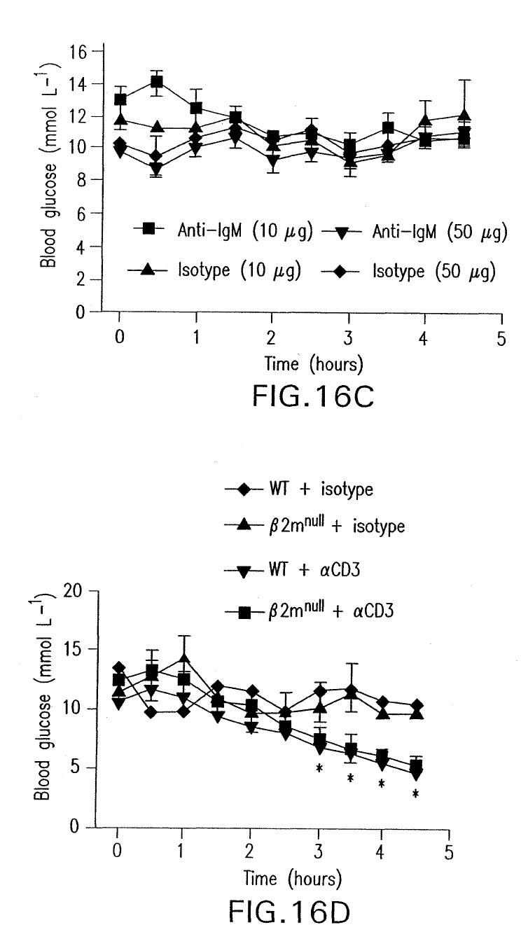

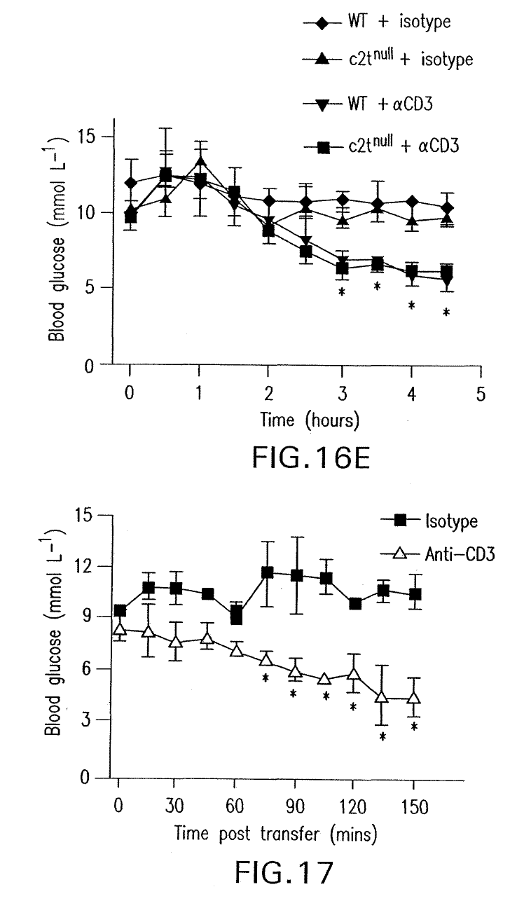

The effects of αCD3 are dependent on T-cells, since αCD3 treatment of DIO-RAG^{null} mice fails to affect glucose levels (see FIG. 16A). B cells are not required since αCD3 is effective in B-cell free (IgM^{null}) mice (see FIG. 16B), and in vivo activation of B-cells with anti-IgM do not affect blood glucose (see FIG. 16C). Interestingly, αCD3 lowers blood glucose in both MHC class I CD8-deficient β₂m^{null} mice and MHC class II CD4-deficient c2t^{null} mice: both CD4+ and CD8+ T-cells appear to have the ability to lower glucose following in vivo activation (FIGS. 16D and 16E). Transfer of (400 μl) serum from αCD3-injected mice 4 hours after i.p. injection into RAG^{null} recipients lowered blood glucose in these recipients (see FIG. 17). This transfer could not be due to residual αCD3 since RAG^{null} animals lack lymphocytes. Serum of αCD3-treated mice became competent to transfer these effects on glucose levels about 3 hours after αCD3 injection. Due to the rapidity of this response, it is contemplated that there may be a molecule(s) or factor(s) that is rapidly up-regulated upon T-cell activation through CD3 that is possibly secreted by one or both of the CD4+ and CD8+ T-cell compartments.

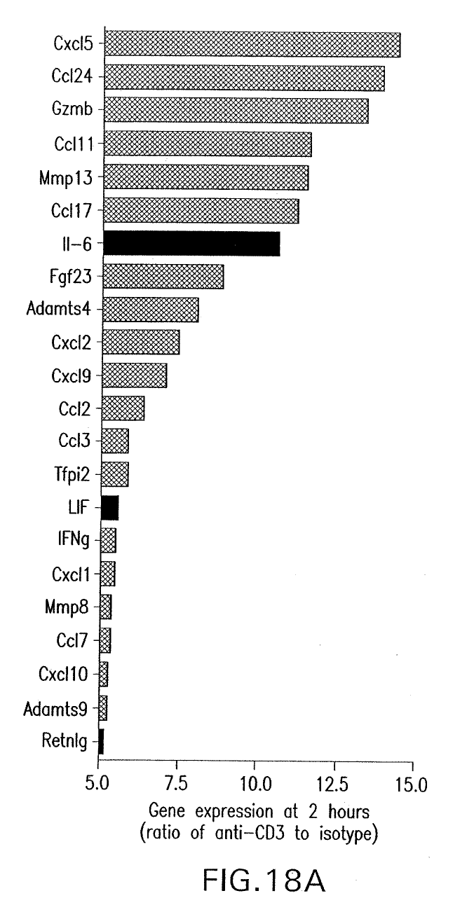

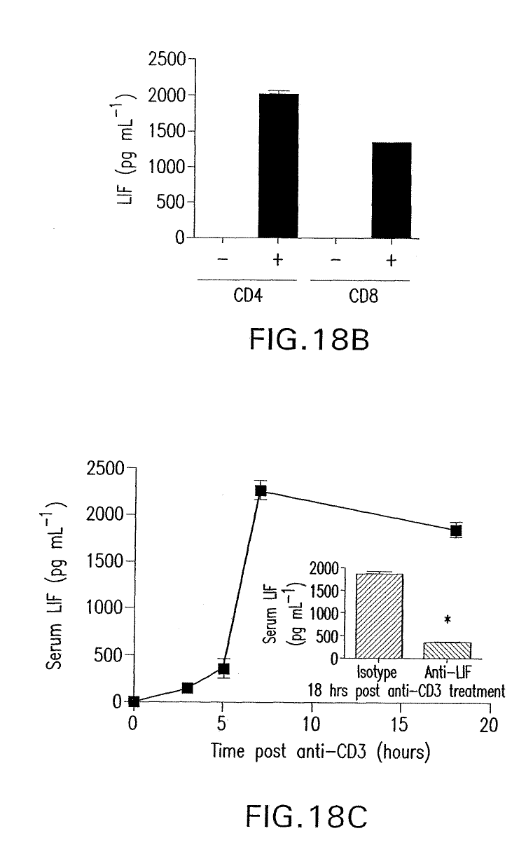

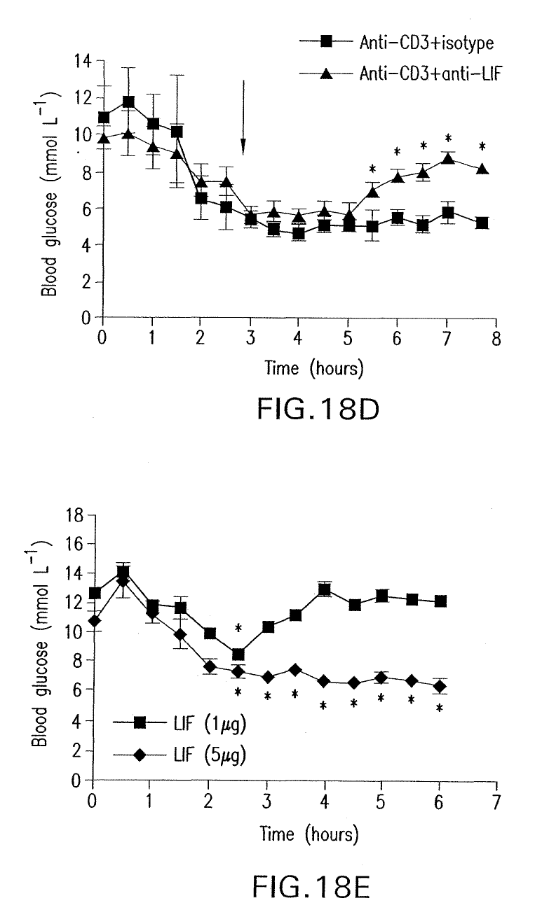

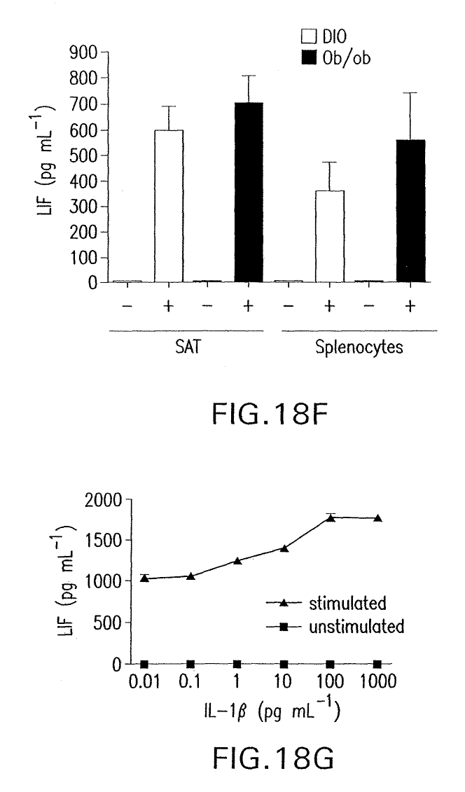

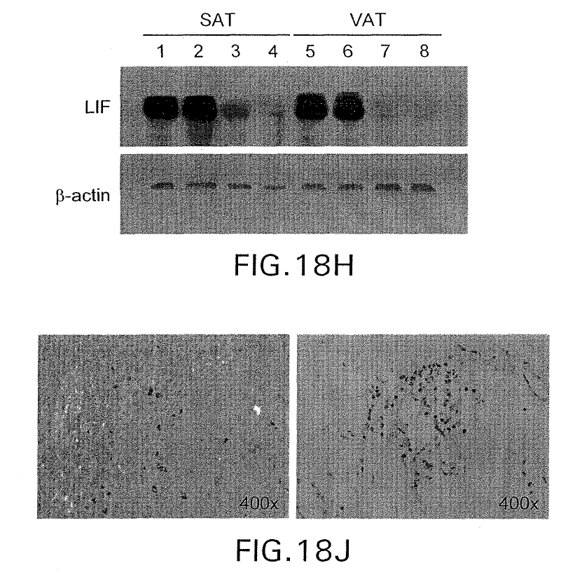

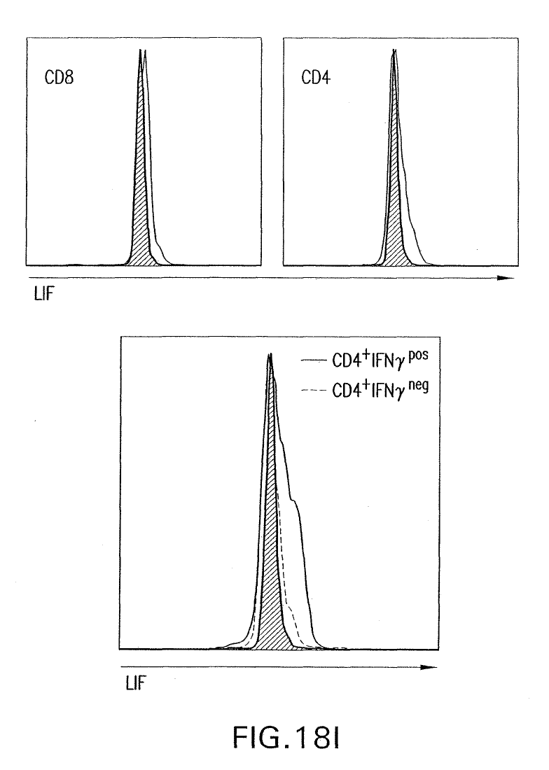

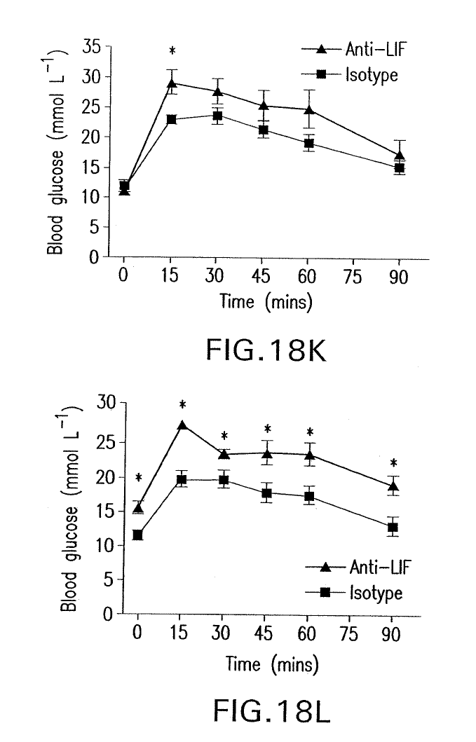

To identify candidate molecules that may mediate the effects of αCD3 treatment, large scale cDNA array screens are performed with spleen cell cDNA taken from mice about 1 and 2 hours after αCD3 injection. As expected, few genes are up-regulated and none more than 2-fold about 1 hour after αCD3 injection. Only 42 genes have greater than about 5-fold increased expression at about 2 hours after αCD3 injection (FIG. 18A). Twenty of these genes do not encode secretory proteins and are therefore removed from consideration. One of secretory protein candidate, Leukemia Inhibiting Factor (LIF), is attractive because of its broadly pleiotropic functions that affect obesity and adipocytes. See, e.g., Jansson, J. O. et al., “Leukemia inhibitory factor reduces body fat mass in ovariectomized mice,” Eur J Endocrinol 154:349-54 (2006); Febbraio, M. A., “A gp130 receptor ligands as potential therapeutic targets for obesity,” J Clin Invest 117:841-9 (2007); and Hogan, J. C. et al., “Effects of leukemia Inhibitory factor on 3T3-L1 adipocytes,” J Endocrinol 185:485-96 (2005), the entire contents and disclosures of which are hereby incorporated by reference. LIF is one of only a few secretory proteins that are expressed by both CD4+ and CD8+ T-cells, and LIF is secreted when plate-bound αCD3 becomes bound to TCR with CD4+ T-cells releasing slightly more LIF than CD8+ T-cells (FIG. 18B).

Following in vivo αCD3 treatment, serum levels of LIF become detectable by about 2.5 hours, reaching a high-level plateau by about 7 hours (FIG. 18C). A single injection of neutralizing anti-LIF antibody at about 7 hours after αCD3 injection reduces serum LIF levels by about 90% at about 7 hours following injection relative to the same time point following injection of an isotype control (see FIG. 18C insert). Earlier injection of anti-LIF (i.e., about 3 hours after in vivo αCD3 treatment) returns blood glucose levels to near the high initial values with a lag time of about 2.5 hours (FIG. 18D). As anticipated from these data, injection of a LIF dose has a similar effect as αCD3 injection in WT DIO mice. LIF i.p. injection reduces starting levels of non-fasting blood glucose in 8 week old WT B6 mice within about 2.5 hours and is sustained with a 5 μg injection relative to starting glucose levels (see FIG. 18E). These independent but mutually supportive observations indicate that αCD3-induced LIF may mediate the beneficial αCD3 effects but do not rule out other possibly less prominent contributions from T-cell proteins or tissue factors.

Considering the DIO-RAG^{null} phenotype, it is important to determine if LIF has a physiological role in glucose homeostasis and/or if it is linked to T^{fat}. Purified T^{fat} produce LIF upon in vitro (plate-bound) αCD3 stimulation of DIO and ob/ob cultured cells (see FIG. 18F), and IL-1β produced a dose-dependent amplification of αCD3-induced LIF production in vitro (FIG. 18G). In vivo, the majority of LIF produced in either SAT or VAT is lymphocyte-derived or lymphocyte-induced because SAT or VAT from RAG^{null} mice produced very little LIF (FIG. 18H). Interestingly, both CD8+ and CD4+ T-cells from DIO WT spleen secrete LIF (about 11% increase stimulation for both with αCD3 stimulation), and many of the LIF-secreting CD4+ T-cells also co-secrete IFNγ (about 25% of CD4+/IFNγ+ T-cells produce LIF while only 6% of CD4+/IFNγ-T-cells produce LIF) (FIG. 18I). Comparable findings are observed for human T^{fat}:immunohistochemistry shows that most T^{fat} co-express CD3 and LIF in human VAT (FIG. 18J, right panel), compared to very few cells in human tonsil (i.e., lymph node) tissue where CD3+ T-cells very rarely co-express LIF (FIG. 18J, left panel). T^{fat} of human VAT show co-staining of LIF and CD3 or CD3 staining around the periphery and LIF staining in the interior of T^{fat} cells.

To test whether LIF actively participates in physiological steady-state control of glucose, neutralizing LIF antibody is injected into DIO mice for about 1 week (FIG. 18K), or for about 2 weeks (see FIG. 18L) into DIO-RAG^{null} mice reconstituted with CD4+ T-cells as before. Even brief anti-LIF treatment reduces glucose tolerance compared to injection of an isotype control, characterizing LIF as a new and physiologically important glucose control agent. These observations suggest a role for T-cells and LIF in limiting obesity and associated MetSyn with remarkable rapidity following αCD3 injection with acute resetting of insulin resistance and a lasting reduction of body weight. These observations provide the possibility for new and improved therapies based on LIF for treatment of obesity, MetSyn, and/or T2D.

### Example 7

### Materials and Methods

Mice. WT C57BL/6 (B6) mice, RAG^{null}, β2m^{null}, c2a^{null}, IgM^{null}, Il-12/p35^{null}, Il-12/p40^{null}, and ob/ob mice, all backcrossed at least 10 generations on C57BL/6 background, are purchased from Jackson Laboratories (Bar Harbor, Me.) and maintained in our vivarium in a pathogen-free, temperature-controlled, 12 hour light and dark cycle environment. B6 “OT2” mice are a kind gift from Dr. Dana Philpott (University of Toronto). Animals are fed either regular diet food or high fat (i.e., about 60 kcal % fat) diet (Research Diets, Inc.). DIO mice receive a regular diet for the first 6 week and then high fat diet for the remainder of experiments. All studies use males under approved protocols and in agreement with animal ethics guidelines.

Cell transfer experiments. Splenocytes from 8 week old C57BL/6 mice on regular diet are isolated as previously described. See, e.g., Winer, S. et al., “Autoimmune Islet Destruction in Spontaneous Type 1 Diabetes is not beta-Cell Exclusive,” Nature Medicine 9:198-205 (2003), the entire contents and disclosure of which are hereby incorporated by reference. CD4+ and CD8+ T-cells are purified as described (i.e., greater than about 95% using an Easy Step negative selection kit; StemCell Technologies, Inc.). For reconstitution studies, 12 week old DIO-RAG^{null} mice receive about 5×10⁶ T-cells intra-peritoneally (i.p.) in about 100 μl of PBS. Lymphocyte trafficking experiments are done as described. See, e.g., Razavi, R. et al., “TRPV1+ sensory neurons control beta cell stress and islet inflammation in autoimmune diabetes,” Cell 127:1123-1135 (2006), the entire contents and disclosure of which are hereby incorporated by reference. 20×10⁶ CFSE-laheled splenocytes from regular diet 8 week old C57BL/6 or C57BL/6 or ob/ob (CellTrace kit, Invitrogen) are injected i.p. and 4 or 7 days later, CFSE+ cells are measured by flow cytometry.

Diet and metabolic studies. All wild-type (WT) and knock-out DIO male mice are weighed about every 1-2 weeks. After about 8 weeks on a high fat diet, fasting blood sugars are determined and insulin levels are measured by ELISA (Crystal Chem, Inc.). For glucose and insulin tolerance testing, mice are fasted for about 16 hours and then injected i.p. with about 0.75 to about 1.0 g of glucose per kg in PBS or injected with about 0.75 or about 2.0 units (U) of human regular insulin (Eli Lilly) per kg in PBS. For VAT:SAT ratios, weights of epididymal (VAT) and inguinal (SAT) fat pads are pooled and averaged. Fat cell diameter is measured microscopically using a straight-line tool of Image SXM v 1.75-1c software. To maintain consistency, all cell diameter measurements are made using the same computer monitor set at pixel dimension of 1360×768 on images taken at 100× the original magnification. At least 200 fat cells from 2 different sections of tissue are quantified for each mouse.

Serum levels of TNF-α, IL-6, adiponectin, leptin, and resistin are measured blindly (AssayGate, Inc.). Levels of phosphorylated-STAT3 and phosphorylated-JNK are determined by ELISA (Multi-Target ELISA kit, Cell Signaling Technologies, Inc.) using 0.5 mg/ml of protein from total homogenates of epididymal and inguinal fat pads as recommended. IL-10 from epididymal and inguinal fat pad homogenates is measured by ELISA (BD Bioscience) and performed as recommended by the manufacturer's protocol.

Purified and recombinant murine LIF (about 1-5 μg) (Chemicon) is injected i.p. in 100 μl of PBS into 8 week old C57BL/6 males. LIF neutralization experiments are performed with i.p. injections of 300 μg goat anti-LIF antibody (R&D Systems) in about 150 μl of PBS, or about 300 μg of normal goat IgG (isotype control, R&D Systems) following indicated schedules.

Oxymax and food intake studies. Mice are placed in individual metabolic chambers with free access to water and pre-weighed food. Oxygen consumption, carbon dioxide output, and heat are measured at 15 min intervals over about 22 hours by indirect calorimetry using the Oxymax System (Columbus Instruments). All measurements are normalized to body weight. Food intake is measured based on the difference between the pre-weighed food given to mice at 0 hours and the same food measured 24 hours later. Studies comparing DIO-WT and DIO-RAG^{null} mice are performed at Jackson Laboratories.

T^{fat} isolation. Mice are anesthetized with inhaled isoflurane and perfused with about 75 ml of PBS through the left ventricle. Epididymal VAT and inguinal SAT pads are dissected with care not to include lymph nodes and ground in a stomacher blending machine. The cell suspension is digested in about 0.2 mg/ml of collagenase (Sigma) in complete DMEM for about 45 min to about 1 hour at about 37° C. with manual resuspension about every 5 minutes. Cells are then filtered through about a 40 to about 70 μm filter or mesh and pelleted by centrifugation to obtain T^{fat}.

Flow cytometry. Splenocytes and/or T^{fat} are blocked (15 min/4° C.) with 10 μg/ml Fc-blocker (eBioscience) and then stained for 30 min with the following dilutions of a given antibody: CD3 (1/150), CD4 (1/200), IL-17 (1/150), IFN-γ (1/100), FOXP3 (1/100), IL-10 (1/100), F4/80 (1/100), MHC Class II (1/100), CD86 (1/100) (eBioscience), CD62L (1/200), CD44 (1/200), CD8 (1/300) (BD Bioscience), or LIF (1/33, R&D, manually labeled with AlexaFluor 647 labeling kit, Molecular Probes). Anti-OVA heavy and light chain antibodies (1/100) are a gift from Dr. Dana Philpott (University of Toronto). For intracellular histological staining of T cells, cells are incubated with PMA (about 50 ng/ml) and ionomycin (750 ng/ml) for about 14 hours in HL-1 media at about 37° C., and golgi are blocked with Golgistop (BD Bioscience) for about the last 11 hours. For intracellular histological staining of macrophages, cells are stimulated with LPS (about 100 ng/ml) for about 6 hours in DMEM with 10% FBS media at about 37° C., and golgi are blocked with Golgistop (BD Bioscience) for the entire 6 hours. FACS plots are analyzed using Flowjo software.

Anti-CD3 experiments. 10 μg of anti-CD3 (clone 145-2C11, BD Biosciences) or purified hamster IgG isotype control (eBioscience) are injected i.p. in 100 μl PBS for 5 consecutive days. For multiple injections, similar doses are injected as indicated. In serum transfer studies, serum is obtained 3-7 hours after anti-CD3 injection via cardiac puncture. 400 μl serum fractions obtained with an Amicon Ultra centrifugal filter devices (Millipore) are injected i.p. into RAG^{null} mice. In B cell studies, B cell activating F(ab′)2 donkey anti-IgM and F(ab′)2 donkey IgG (Jackson ImmunoResearch) are used as described. For cDNA gene arrays, RNA from spleen obtained about 1 or 2 hours after a single injection of 10 μg of anti-CD3 is isolated using an RNeasy kit (Qiagen). Gene arrays are conducted using the Mouse Gene 1.0 ST array (Affymetrix).

Histology, Immunohistochemistry, and human tissue. Liver, VAT and SAT fat pads from various test animals are removed and fixed in 10% buffered formalin for about 24 hours. H&E staining is performed on paraffin embedded sections and analyzed blindly by 2 people, including one certified pathologist (D.W.). For human tissue, VAT is obtained from mesenteric fat from surgically removed colons of overweight colon cancer patients. Immunohistochemistry uses paraffin sections subjected to antigen retrieval in pH 6 Diva Decloaker universal antigen retrieval solution (Biocare Medical) with the following antibody dilutions: 1/100 mouse anti-FOXP3 (Abeam), 1/100 rabbit anti-T-bet (Santa Cruz), 1/100 rabbit anti-CD14 (Atlas), 1/200 mouse anti-LIF (R&D), and 1/50 rabbit anti-CD3 (Vector Labs). Color is developed with DAB solution (Vector labs) and Ferangi blue solution (Biocare Medical). T-bet:Foxp3 ratios are obtained by counting at least 200 stained cells from 2 different levels of tissue. There is no tumor infiltration into areas of VAT examined.

Microscopic images are taken with a V2.4 Nuance Multispectral Imaging VIS Flex Camera system (CR1) using the Multiple Molecular Marker Plugin (CR1). The positivity threshold for LIF is set to 0.450 and 0.300 for CD3. False color LIF staining is in red, and CD3 staining is in green with co-localizing signals yellow.

For anti-LIF Western blots, 50 μg of total fat pad protein homogenized from epididymal VAT or inguinal SAT in RIPA lysis buffer containing protease inhibitor and phosphatase inhibitor (Santa Cruz) before probing with 1/500 polyclonal goat anti-LIF (R&D systems) or 1/1000 rabbit anti-β-actin (Cell Signaling).

Statistical analysis. Statistical significance between two means is assessed by Mann-Whitney and unpaired Welch-tests. Welch correction on t-tests is employed for sample sizes less than 6. Analysis of curves is performed using Two-Way ANOVA. Statistical significance is two tailed and set at 5%.

### Remarks

A fundamental role for CD4+ T lymphocytes in the regulation of body weight, adipocyte hypertophy, hepatosteatosis, and insulin-resistance is uncovered, thus identifying physiological controls that counteract disease progression in DIO. Several independent observations support the involvement of T lymphocytes. In particular, RAG^{null} mice develop an “almost diabetic” phenotype on a regular diet but develop an unrestrained and excessive visceral obesity and insulin-resistance on a high fat diet. Adoptive T-cell transfer studies and the effectiveness of αCD3 in treating disease states map T-cell mediated metabolic rescue to CD4+Foxp3− and CD4+Foxp3+ T-cell compartments. For example, Th2 cells emerge as potential regulators of obesity and insulin resistance in the CD4+ transfer model. However, the function of these cells in wild type mice or humans has not been explored these examples are not designed to rule out any additional effects of B lymphocytes or VAT-associated innate lymphocyte subsets (e.g., NK/NKT, TCRγ/δ, CD4−/CD8− TCRα/β+). See, e.g., Caspar-Bauguil, S. et al., “Adipose tissues as an ancestral immune organ: site-specific change in obesity,” FEBS Lett 579:3487-3492 (2005), the entire contents and disclosure of which are hereby incorporated by reference.

Limited studies in humans closely agree with conclusions deduced from the mouse model data. VAT of DIO mice and obese humans both appear to be characterized by a higher Th1:Foxp3+ ratio than in VAT of regular diet mice or normal weight humans. High expression of IFNγ in human T^{fat} has been associated with increased waist circumference. See, e.g., Kintscher, U. et al., supra (2008). IFNγ is a pro-inflammatory cytokine, and increased levels of Th1 cells in VAT may contribute to insulin resistance. This observation may be supported by improved glucose tolerance in Th1 cell deficient IL-12/p35^{null} mice (Winer et al. unpublished observation) and IFNγ^{null} mice. See, e.g., Rocha, V. Z. et al., “Interferon-gamma, a Th1 cytokine, regulates fat inflammation: a role for adaptive immunity in obesity,” Circ Res 103:467-476 (2008), the entire contents and disclosure of which are hereby incorporated by reference.

Although CD4+Foxp3+ T-cells appear to mitigate abnormal glucose control in DIO, the ratio between pro-inflammatory Th1 and anti-inflammatory CD4+Foxp3+ T cells emerges as a likely factor in determining disease progression and outcomes. However, the absolute number of VAT-associated CD4+Foxp3+ T-cells appears to change very little in DIO, while the absolute number of VAT-associated Th1 cells increases dramatically in these mice. A model is proposed herein where a relatively constant pool of CD4+Foxp3+ T^{fat} gradually fails to counter-regulate increasingly overwhelming pools of Th1 T cells, which lead to a progressively pro-inflammatory environment that promotes insulin resistance. Factors that drive influx and/or expansion of Th1 cells in VAT are unknown but may include cognate events. A prevalence of Th1 cells in human adipose tissue from obese patients has been noted. See, e.g., Wu et al., supra (2007).

The precise mechanisms by which CD4+ T-cells affect insulin-sensitivity may require further study. The induction of IL-10-secreting AAMs is a mechanism observed. CD4+Foxp3+ T-cells can induce M2c type AAMs, while Th2 T cells can induce M2a type AMMs. See, e.g., Martinez, F. O. et al., supra (2008). AAMs may regulate insulin resistance though production of IL-10-mediated reversal of TNFα-induced insulin resistance. See, e.g., Lumeng, C. N. et al., supra (2007). AAMs are present in adipose tissue of lean mice, but as obesity ensues, there is a phenotypic shift to pro-inflammatory M1 macrophages. This shift to M1 macrophages is likely a result of steadily increasing Th1:Foxp3 ratios that progressively overwhelm the CD4+ anti-flammatory T-cell mediated disease control via alternative production of AAM. Therefore, strategies increasing AAM or cells that induce AAM (e.g., CD4+Foxp3+ T cells) may provide therapeutic promise for the treatment of MetSyn and T2D. In fact, increases in VAT M2 and decreases in M1 macrophages following restoration of CD4+Foxp3+ T cell pools following F(ab′)₂ immunotherapy are observed.

Short term treatment with either αCD3 or its F(ab′)₂ fragment raised the Foxp3+ T cell pools in VAT and normalized glucose levels and insulin sensitivity without changes in feeding behavior or body weight over time, thus highlighting the therapeutic potential of these treatments. αCD3 is a potent, clinically used immune-suppressant and a possible therapeutic for Type 1 Diabetes. See, e.g., Chatenoud, L. et al., supra (2007); and Herold, K. C. et al., “Anti-CD3 monoclonal antibody in new-onset type 1 diabetes mellitus,” N Engl J Med 346:1692-1698 (2002), the entire contents and disclosures of which are hereby incorporated by reference. The effects of αCD3 on immunity are reversible and well understood since αCD3 therapy has been clinically used in other contexts. A novel use for αCD3 therapy is provided herein for the treatment of insulin resistance associated with MetSyn and T2D. Use of αCD3 or its F(ab′)₂ fragment in these patients may have multiple benefits since αCD3 is also effective in reducing atherosclerotic plaques common in obesity and insulin resistance syndromes. See, e.g., Steffens, S. et al., “Adiponectin and adaptive immunity: linking the bridge from obesity to atherogenesis,” Circ Res 102:140-142 (2008), the entire contents and disclosure of which are hereby incorporated by reference.

Current focus has been on the metabolic roles of lymphocytes in VAT, but the impact of lymphocytes on insulin resistance in other organs such as liver cannot be ruled out. Indeed, RAG^{null} mice display marked hepatic steatosis that may be reversed by CD4+, but not CD8+, T-cell transfer. A potential role for CD8+ T cells in disease development also cannot be entirely ruled out. Although transfer studies show that CD8+ T cells by themselves appear to play a small role in obesity and associated insulin resistance, it may be possible that CD8+ T-cells, and possibly regulatory CD8+CD25+ the subset, may play a cooperative role with CD4+ T-cells. See, e.g., Bisikirska, B. et al., supra (2005).

A previously unknown role for the adaptive immune system in the regulation of obesity, fat distribution, and insulin-resistance is provided. Both CD4+Foxp3− and CD4+Foxp3+ T-cell populations are shown to function as physiological regulators of these metabolic processes, and a VAT-selective and TCR-dependent (i.e., cognate) form of autoimmunity appears to be at least one element driving T-cell expansion in VAT. VAT-specific T-cell expansion with secondary TCRα rearrangements and uniquely biased TCR-Vα clones in VAT suggest that one or more VAT antigens may help to trigger a form of T-cell autoimmunity which may ultimately drive the progressive expansion of the VAT lesion in MetSyn and T2D. Furthermore, the observation of a strongly biased TCRα usage in VAT T-cells from wild-type DIO B6 mice suggests that obesity and/or T2D (along with their associated metabolic syndromes) may be amenable to treatment using affordable tolerogenic vaccination strategies once the driving auto-antigen(s) of these diseases are identified.

A local and perhaps systemic role for LIF-expressing T-cells in altering fat accumulation and distribution, euglycemia, insulin-sensitivity, etc. is provided. Adipocytes and pre-adipocytes express LIF receptors, and one report identified Foxp3+ T-cells as a source of LIF. See, e.g., Hogan, J. C. et al., “Effects of leukemia Inhibitory Factor on 3T3-L1 adipocytes,” J Endocrinol 185:485-96 (2005); and Metcalfe S. M. et al., “Leukemia inhibitory factor is linked to regulatory transplantation tolerance,” Transplantation 79:726-30 (2005), the entire contents and disclosures of which are hereby incorporated by reference. LIF may signal through STATS and, to a lesser extent, STAT-1, with STAT-3 signaling linked to reduced insulin-resistance. See, e.g., Stephens, J. M. et al., “Activation of signal transducers and activators of transcription 1 and 3 by leukemia inhibitory factor, oncostatin-M, and interferon-gamma in adipocytes,” J Biol Chem 273:31408-16 (1998), the entire contents and disclosure of which are hereby incorporated by reference. Analysis of the effects of injected LIF and LIF-blockade by a neutralizing anti-LIF antibody, shows that LIP can regulate glucose homeostasis in WT DIO mice and in T-cell reconstitution models.

The mechanism of action for LIF may be pleiotropic. Most current interest in the effects of LIF have focused on stem cell physiology (see, e.g., Metcalf, D., “The unsolved enigmas of leukemia inhibitory factor,” Stem Cells 21:5-14 (2003), the entire contents and disclosure of which are hereby incorporated by reference), but there is some evidence that LIF may affect lipolysis and triglyceride accumulation. See, e.g., Hogan, J. C. et al., supra (2005); and Marshall, M. K. et al., “Leukemia inhibitory factor induces changes in lipid metabolism in cultured adipocytes,” Endocrinology 135:141-7 (1994), the entire contents and disclosures of which are hereby incorporated by reference. Although it may be likely that LIF exerts its effect directly in adipose tissue, there may well be additional systemic and central nervous system actions of LIF. See, e.g., Watt, M. J. et al., “CNTF reverses obesity-induced insulin resistance by activating skeletal muscle AMPK,” Nat Med 12:541-8 (2006); and Beretta, E. et al., “Central LIF gene therapy suppresses food intake, body weight, serum leptin and insulin for extended periods,” Peptides 23:975-84 (2002), the entire contents and disclosures of which are hereby incorporated by reference. For example, it is possible that other T-cell derived cytokines may participate in LIF-dependent or independent effects on glucose tolerance and weight loss, but LIF by itself has been shown to have a major impact on metabolic parameters which reproduce many of the effects of αCD3 treatment.

LIF potently inhibits macrophage release of IL-6 and TNFα. See, e.g., Weber, M. A. et al., “Endogenous leukemia inhibitory factor attenuates endotoxin response,” Lab Invest 85:276-84 (2005), the entire contents and disclosure of which are hereby incorporated by reference. In an adipose tissue environment, T-cells, which may be selected by local adipose antigens and/or fueled by elevated IL-1β in fat (See, e.g., Zeyda, M. et al., “Human adipose tissue macrophages are of an anti-inflammatory phenotype but capable of excessive pro-inflammatory mediator production,” Int J Obes (Lond) 31:1420-8 (2007), the entire contents and disclosure of which are hereby incorporated by reference), may increase LIF expression and improve insulin-sensitivity through inhibition of macrophage activation and increased STAT3 signaling. This scenario is supported by observations of IL-1β enhanced LIF production, raised STAT3 and reduced JNK phosphorylation following CD4+ T-cell transfer into RAG^{null} mice, and increased levels of MCP-1, IL-6, and TNFα along with lower levels of LIF in RAG^{null} mice.

A new and previously unknown role for T-cells in the regulation of obesity, insulin-resistance, and MetSyn progression in engineered mouse models is provided, which may also be applicable to humans since, for example, human VAT has very similar T-cell constituents as obese mice, including the fat-selective increase in LIF+ Th1 cells. Observations from these studies indicate that LIF, αCD3 antibody, or engineered fragments of αCD3 may have considerable promise in the treatment of obesity, MetSyn, and/or T2D. Indeed, treatment of DIO mice with αCD3 antibody resulted in rapid euglycemia and improved glucose- and insulin-sensitivity with long-term reduction in body weight. It is further envisioned that any known or later developed small molecules and other effector molecules that affect the same pathways as LIF, αCD3 antibody, or engineered fragments thereof, may also be used for treatment of these conditions. Since αCD3 is already used clinically as an immunosuppressant (see, e.g., Herold K. C. et al., supra (2002); and Chatenoud, L. et al., supra (2007)), αCD3 may provide immediate clinical utility in treating these conditions, which may be used, for example, in place of invasive and difficult-to-reverse bariatric surgery in treating obesity.

While the present invention has been disclosed with references to certain embodiments and examples, numerous modifications, alterations, and changes to the described embodiments are possible without departing from the sphere and scope of the present invention, as defined in the appended claims. Accordingly, it is intended that the present invention not be limited to the described embodiments or examples, but that it has the full scope defined by the language of the following claims, and equivalents thereof.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

29. (canceled)

30. (canceled)

31. (canceled)

32. (canceled)

34. (canceled)

35. (canceled)

36. (canceled)

37. (canceled)

38. A use of a T cell activating antibody to treat an individual having, or at risk of developing, metabolic syndrome (MetSyn) or Type-2 diabetes (T2D),
wherein the individual having, or at risk of developing, MetSyn or T2D is an obese human individual.

39. The use of claim 38, wherein the obese human individual has a body mass index (BMI) of about 30 or greater.

40. The use of claim 38, wherein the individual having, or at risk of developing, MetSyn or T2D is an overweight human individual.

41. The use of claim 40, wherein the overweight human individual has a body mass index (BMI) in a range of about 25 to about 30.

42. The use of claim 38, wherein the individual having, or at risk of developing, MetSyn or T2D is an individual having an abnormally elevated ratio of Th1 T-cells to Foxp3+ T-cells in adipose tissue.

43. (canceled)

44. (canceled)

45. (canceled)

46. The use of claim 38, wherein the individual having, or at risk of developing, MetSyn or T2D is a human individual having T2D.

47. The use of claim 46, wherein the human individual having T2D has a fasting blood glucose level of about 7.0 mmol per liter or greater prior to step (b).

48. The use of claim 46, wherein the human individual having T2D has a blood glucose level in a range of about 11.0 mmol per liter or greater two hours after ingesting a 75-gram glucose drink in an oral glucose tolerance test (OGTT) prior to step (b).

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)
